(19) United States
(12) Patent Application Publication
(10) Pub. No.: US 2004/0197777 A1
(43) Pub. Date: Oct. 7, 2004

(54) Polymorphisms of the OCTN1 and OCTN2 cation transporters associated with inflammatory bowel disorders

(76) Inventors: Vanya D. Peltekova, Toronto (CA); Richard F. Wintle, Toronto (CA); Laurence A. Rubin, Toronto (CA); Peter H. St George-Hyslop, Toronto (CA); Katherine A. Siminovitch, Toronto (CA)

Correspondence Address: McDERMOTT, WILL & EMERY, 28 State Street, Boston, MA, 02109-1775, US

(21) Appl. No.: 10/327,188

(22) Filed: Dec. 20, 2002

Related U.S. Application Data
(60) Provisional application No. 60/343,338, filed on Dec. 21, 2001.
(60) Provisional application No. 60/362,700, filed on Mar. 8, 2002.
(60) Provisional application No. 60/362,717, filed on Mar. 8, 2002.
(60) Provisional application No. 60/427,529, filed on Nov. 19, 2002.

(51) Int. Cl. C12Q 1/68; C07H 21/04

(52) U.S. Cl. 435/6; 536/23.2; 435/69.1; 435/320.1; 435/325; 530/350

(57) ABSTRACT

The invention provides a method for diagnosing Inflammatory Bowel Diseases, using genetic markers that are implicated in severe, early-onset Crohn's Disease (CD). The invention also provides coding sequence mutations in the OCTN1 gene that significantly reduces its ability to transport the organic cation carnitine. The invention further provides mutations in the promoter region of OCTN2 (that downregulates both basal transcription and transcription induced either by heat shock or arachidonic acid. This transcription difference is apparently due to the disruption of a binding site for heat shock transcription factor 1 (HSF1). A haplotype of two mutations is found in Crohn's Disease (CD) patients. Together, these mutations reduce cellular ability to respond to metabolic stress in inflamed tissue and may further be involved in the clearance of toxic substances from cells. The invention provides for the identification and use of these polynucleotides and encoded polypeptide sequences as targets for the development of therapeutic compounds intended to be useful in the treatment of Inflammatory Bowel Diseases and inflammation generally.

### CLAIM OF PRIORITY

This patent claims priority to U.S. provisional patent applications Ser. Nos. 60/343,338, filed Dec. 21, 2001; 60/362,700, filed Mar. 8, 2002; 60/362,717, filed Mar. 8, 2002; and 60/427,529, filed Nov. 19, 2002.

### FIELD OF THE INVENTION

This invention relates generally to the analysis of biological material. In particular, the invention relates to diagnostic analyses for markers that are associated with inflammatory disorders of the gastrointestinal tract, and to their uses for the development of novel therapeutic treatments for inflammatory disorders.

### BACKGROUND OF THE INVENTION

Crohn's Disease (CD) is an idiopathic condition characterized by chronic, relapsing intestinal inflammation, with most patients developing symptoms in early adulthood. It is a subset of the Inflammatory Bowel Diseases (IBD) which include Crohn's Disease, indeterminate colitis (IC) and Ulcerative Colitis (UC).

These conditions affect a relatively large portion of the population, with varying estimates of prevalence around 300,000 in North America. Inflammatory Bowel Diseases have a high economic impact, with costs running into the billions of dollars.

Genetic studies have indicated the presence of several loci predisposing to Inflammatory Bowel Diseases, the most widely replicated of which are on chromosomes 16 (caused by mutations in the NOD2/CARD15 gene), 12, 6 and 14. A locus at chromosome 5q31, termed IBD5, which predisposes to early-onset Crohn's Disease (with age of onset 16 years or earlier) has been described by Rioux J D et al., Nature Genet. 29, 223-228 (2001). This locus is marked by a risk haplotype of 11 Single Nucleotide Polymorphisms (SNPs) over a region of approximately 250-kb. The region contains multiple candidate genes including the genes for organic cation transporters (OCTN1/SLC22A4 and OCTN2/SLC22A5), the gene for a LIM-domain-containing protein (RIL/PDLIM3), the gene for the α2 subunit of proline-4-hydroxylase (P4HA2) and a gene of unknown function (NCBI UniGene identifier Hs.70932). Because of extensive linkage disequilibrium (LD) in this region, it had not previously been possible to further refine the SNP map and unambiguously identify a single susceptibility gene.

Thus, there is a continuing need in the medical arts for genetic markers of Crohn's Disease and guidance for the use of such markers.

### SUMMARY OF THE INVENTION

The invention provides a method for diagnosing Inflammatory Bowel Diseases, using genetic markers that are implicated in severe, early-onset Crohn's Disease (CD). The invention also provides coding sequence mutations in the OCTN1 gene (the human OCTN1 gene, also known as “Homo sapiens solute carrier family 22 (organic cation transporter), member 4 (SLC22A4)”) that significantly reduces its ability to transport the organic cation carnitine. The invention further provides mutations in the promoter region of OCTN2 (the human OCTN2 gene, also known as “Homo sapiens solute carrier family 22 (organic cation transporter), member 5 (SLC22A5)”) that downregulates both basal transcription and transcription induced either by heat shock or arachidonic acid. This transcription difference is apparently due to the disruption of a binding site for heat shock transcription factor 1 (HSF1). The invention further provides a haplotype of these two mutations in combination. The two genes, OCTN1 and OCTN2, show significant downregulation in inflamed Crohn's Disease tissue.

The identified OCTN1 and OCTN2 sequence variations have diagnostic and pharmacogenetic utility and reveal a disease-related molecular pathway that can be targeted for therapeutic intervention. The OCTN1 and OCTN2 genes are differentially expressed in inflamed and non-inflamed colon tissue from CD patients. Both genes contain disease-associated DNA sequence variations (polymorphisms). The identified polynucleotide polymorphisms are thus the basis for a diagnostic and prognostic test describing susceptibility to Inflammatory Bowel Diseases in certain individuals. The invention also provides for the identification and use of these polynucleotides and encoded polypeptide sequences as targets for the development of therapeutic compounds intended to be useful in the treatment of Inflammatory Bowel Diseases and inflammation generally.

The invention also provides a model for Crohn's Disease, wherein the combined effect of these two mutations results in overall poor carnitine transport, particularly in response to inflammation. As carnitine is a cofactor required for the uptake of long-chain fatty acids into the mitochondria for subsequent β-oxidation, the effect of this mutation haplotype leads to metabolic stress, which contributes to inflammatory damage to gastrointestinal tract tissue in Crohn's Disease patients. In addition, the invention provides evidence of an association between the OCTN1 and OCTN2 genes and generalized inflammatory responses. This model provides the basis for the therapeutic treatment of Inflammatory Bowel Diseases. The invention also provides a model for Crohn's Disease, wherein the effect of mutations in OCTN1 results in overall poor efflux of molecules from the cell. These molecules could include toxic metabolites, bacterial endotoxins, xenobiotics, pharmaceutical compounds used to treat inflammation, or free radical species.

### BRIEF DESCRIPTION OF THE DRAWINGS

FIG. 1 is a set of Northern blots showing a differential expression of OCTN1 and OCTN2 in inflamed tissue. Northern blots probed with OCTN1, OCTN2 or GAPDH are shown at the left of FIG. 1. Bar graphs of the relative optical density (normalized to GAPDH) corresponding to OCTN1 expression (right side, above) or OCTN2 expression (right side, below) are shown at the right, with value±SEM labeled on the bar. p-values of significance are shown above the bar corresponding to inflamed tissue. HC: normal human colon. J: Jurkat T-cell leukemia cells. UC-N: non-inflamed tissue from Ulcerative Colitis patient. UC-I: inflamed tissue from Ulcerative Colitis patient. CD-N: non-inflamed tissue from Crohn's Disease patient. CD-I: inflamed tissue from Crohn's Disease patient.

FIG. 2 is a set of bar graphs showing that a G-207C mutation in the heat shock enhancer (HSE) of the promoter region of OCTN2 gene results in downregulation of luciferase reporter gene under heat shock and arachidonate treatment. HeLa cells transfected with constructs containing the luciferase reporter gene fused to the 2.7-kb region of OCTN2 promoter with wild-type (G-207) or mutant (G-207C) alleles. Relative luminescence of transfected OCTN2 promoter constructs was measured in untreated control cells (37° C.), cells exposed to a standard heat shock (42° C.) and to 20 μM arachidonic acid (arachidonate). Values are means of three independent experiments. Error bars are the SEM. G: nonrisk allele at position −207 of the OCTN2 promoter; C: risk allele.

FIG. 3 is a set of bar graphs showing the carnitine transport by human HeLa (FIG. 3A) and human OCTN2 deficient cell line (FIG. 3B), transfected with OCTN1 cDNA with L503 or the mutant L1503F allele. Cells transfected with pcDNA3 vector alone served as a control. Uptake of [³H]-carnitine (20 nM) was measured at pH 7.5 in a media containing NaCl. Carnitine uptake was calculated as cpm/mg protein/min, values were corrected for transfection efficiency and the uptake was normalized with respect to the value of the mock transfected cells. Points are means±SE of three (FIG. 3A) to six (FIG. 3B) determinations in three (FIG. 3A) or two (FIG. 3B) independent experiments.

FIG. 4 is a set of bar graphs showing TEA efflux by the mutant 503F and wild-type 503L variants of OCTN1. Intracellular retention of radiolabeled TEA is shown as a percentage of the maximally retained amount at time zero. OCTN1-F: L503F variant of OCTN1. OCTN1-L: wild-type 503L variant of OCTN1. Points are means±SE of three determinations.

FIG. 5 is a set of bar graphs showing the luciferase activities of the pRL-null vector containing 2.7 kb OCTN2 promoter region.

FIG. 6 shows the genomic DNA sequence including the OCTN2 gene. Locations of exons are highlighted in yellow. Locations of primer sequences used are highlighted in green (note that the O2X1F primer is located within exon 1, and that O2X1R is the reverse complement of the sequence as shown). Start and stop codons of the coding sequence are highlighted in red. Mutations within the 5′ region of the gene are highlighted and are shown with standard DNA nomenclature for polymorphic bases, i.e. “K” meaning G or T, “Y” meaning C or T, “R” meaning A or G, and “S” meaning C or G.

FIG. 7 shows in graphical form the expression level of the OCTN2 gene, expressed as relative optical density (“ROD”) of autoradiographic signal from the OCTN2 gene as compared to the glyceraldehyde-3-phosphate dehydrogenase (GAPDH) gene. Blue bars represent expression level from RNA extracted from surgically removed non-inflamed GI tract tissue, and red bars represent expression level from RNA extracted from surgically removed inflamed GI tract tissue. Individual RNA samples are identified below the bars with a unique identifier number (e.g. 7403) and patient's diagnosis (“UC” for Ulcerative Colitis, “CD” for Crohn's Disease). Control unaffected samples are labeled as “HC male” and “HC female” and are from individuals without Inflammatory Bowel Disease. FIGS. 7A-D each represent data from a single “Northern blot” (i.e. DNA:RNA hybridization experiment); for each blot, individual sample results are presented in the top panel and averaged results for inflamed and non-inflamed samples are presented in the bottom panel. In the bottom panel, each bar is labeled with its mean value and standard error of the mean (SEM). Statistical p-values are also presented in the bottom panel. FIG. 7E shows individual sample results for pairs of inflamed and non-inflamed tissue samples from the same individual. Individual identifier numbers and diagnoses, values and standard errors are indicated as in FIGS. 5A-F shows the averaged results for all affected and unaffected samples. Individual identifier numbers and diagnoses, values and standard errors are indicated as in FIGS. 7A-D.

FIG. 8 shows the genomic DNA sequence including the OCTN1 gene. Locations of exons are highlighted in yellow. Locations of oligonucleotide primer sequences used are highlighted in green (note that the O1X9R primer is the reverse complement of the sequence as shown). Start and stop codons of the coding sequence are highlighted in red. The mutation within exon 9 of the gene is highlighted and is shown with standard DNA nomenclature for polymorphic bases, i.e. “Y” meaning C or T.

FIG. 9 shows the amino acid sequence as determined from known mRNA sequences for OCTN1. Each letter represents a single amino acid as defined by standard nomenclature. The location of the amino acid change caused by the OCTN1 exon 9 mutation is shown as “(L/F)”, meaning that where the “C” allele is present in genomic DNA, Leucine (“L”) is the corresponding amino acid in the OCTN1 protein, and where the “T” allele is present in genomic DNA, Phenylalanine (“F”) is the corresponding amino acid in the OCTN1 protein.

FIG. 10 shows in graphical form the expression level of the OCTN1 gene, expressed as relative optical density (“ROD”) of autoradiographic signal from the OCTN1 gene as compared to the glyceraldehyde 3-phosphate dehydrogenase (GAPDH) gene. Blue bars represent expression level from RNA extracted from surgically removed non-inflamed GI tract tissue, and red bars represent expression level from RNA extracted from surgically removed inflamed GI tract tissue. Individual RNA samples are identified below the bars with a unique identifier number (e.g. 7403) and patient's diagnosis (“UC” for Ulcerative Colitis, “CD” for Crohn's Disease). Control unaffected samples are labeled as “HC male” and “HC female” and are from individuals without Inflammatory Bowel Disease. FIGS. 10A-C each represent data from a single “Northern blot” (i.e. DNA:RNA hybridization results); for each blot, individual sample results are presented in the top panel and averaged results for inflamed and non-inflamed samples are presented in the bottom panel. In the bottom panel, each bar is labeled with its mean value and standard error of the mean (SEM). Statistical p-values are also presented in the bottom panel. FIG. 10D shows individual sample results for pairs of inflamed and non-inflamed tissue samples from the same individual. Individual identifier numbers and diagnoses are indicated as in FIGS. 10A-C. FIG. 10E shows averaged results for all affected and unaffected samples. Individual identifier numbers and diagnoses, values and standard errors are indicated as in FIGS. 10A-C.

### DETAILED DESCRIPTION OF THE INVENTION

Summary. A functional analysis and in-depth mutation screening of candidate genes was performed in the 250 kb region of chromosome 5q31, described by Rioux J D et al., Nature Genet. 29, 223-228 (2001). Also examined were genes in the region for expression level differences in inflamed and non-inflamed gastrointestinal (GI) tract tissue from Inflammatory Bowel Disease patients. Two genes, OCTN1 (SEQ ID NO:1) and OCTN2 (SEQ ID NO:3), showed significant downregulation in inflamed Crohn's Disease tissue. A coding sequence mutation in the OCTN1 gene that significantly reduces its ability to transport the organic cation carnitine and the prototypical organic cation substrate tetraethyl ammonium (TEA) was detected. Additionally, a mutation in the promoter region of OCTN2 was found to downregulate both basal transcription and transcription induced either by heat shock or arachidonic acid. This transcription difference is apparently due to the disruption of a binding site for heat shock transcription factor 1 (HSF1).

Mutation detection. For mutation detection and analysis, bidirectional sequencing of polymerase chain reaction (PCR) amplified exons of the OCTN1 and OCTN2 genes was performed on ABI 377 or 3700 sequencers. The detected mutations were verified by visual inspection of both forward and reverse electropherograms. PCR primers for genomic DNA were designed based on the genomic sequences of human OCTN1 and OCTN2 reported in GenBank (loci AC008599 and AC004628 on chromosome 5q). Primers were ˜60 bp upstream or downstream of each exon, allowing sequencing of splice donor and splice acceptor sequences and of the lariat branch site. In the case of the OCTN2 promoter region, PCR amplification of 1.3 kb fragment using PCR primers, two primers, OCTN2 Promoter Forward (O2PF): 5′-CTGCACGGAAATAACTAATCTGTG-3′ (SEQ ID NO:5) and OCTN2 Promoter Reverse (O2PR): 5′-GAGAGGAGCTCGGGTTCAAG-3′ (SEQ ID NO:6) were used. PCR was performed using PCRx Enhancer System (Invitrogen, La Jolla, Calif., USA) with addition of 1× final PCRx Enhancer solution concentration, to overcome the GC-rich nature of this region, and with Platinum Taq DNA Polymerase High Fidelity (Invitrogen, La Jolla, Calif., USA). The PCR protocol includes 30 cycles of 94° C (for 40 sec), 54° C. (for 1 min), and 72° C. (for 2 min) with a 5-min extension at 98° C. at the first cycle and a 10-min extension at 72° C. of the final cycle.

SNP risk haplotypes were determined from data previously described SNP by Rioux J D et al., Nature Genet. 29: 223-228 (2001), and OCTN1 and OCTN2 mutation data were phased with SNP haplotypes by visual inspection of pedigrees. Significance levels for allele frequency differences between groups (see, TABLE 1, below) were calculated with a two by two chi-squared contingency table. Relative risk calculations (formally, odds ratios) were calculated as described by Bland J M & Altman D G, BMJ 320: 1468 (2000).

Mutations were initially detected by sequencing of exons, splice junctions and promoter regions of the OCTN1 and-OCTN2 genes from a panel of 16 unrelated patients either heterozygous or homozygous for the Crohn's Disease SNP risk haplotype at 5q31.

Two mutations that co-segregated with the SNP risk haplotype were identified, a C to T change in exon 9 of OCTN1 that changes the leucine residue at position 503 to phenylalanine (L503F; SEQ ID NO:7), and a G to C change 207 bp 5′ of the start codon of OCTN2 (G-207C; SEQ ID NO:8). The G-207C mutation alters a consensus heat shock transcription factor 1 (HSF1) binding site. Sequencing of other candidate genes in the region did not reveal any mutations consistently found on the risk haplotype background.

We then sequenced exon 9 of OCTN1 and the promoter region of OCTN2 in individuals from 46 families that were used for previous SNP analysis. Rioux J D et al., Nature Genet. 29, 223-228 (2001). Forty-one of these families segregate at least one copy of the SNP haplotype, and five segregate no copies of this haplotype. Examination of SNP haplotypes in unrelated children from these families revealed that the previously defined SNP risk haplotype always carried both the phenylalanine variant of L503F and the C allele of G-207C.

We next genotyped these two mutations in a panel of healthy controls, and in a panel of patients known to carry no copies of the SNP risk haplotype. The L503F (SEQ ID NO:7) and G-207C (SEQ ID NO:8) mutant alleles were significantly more common in unrelated patients from these families than in healthy controls, and were almost never found in patients homozygous for the nonrisk haplotype (only one L503F allele in 25 patients (50 chromosomes) and one G-207C allele in 16 patients (32 chromosomes). Allele frequencies in the control group did not deviate from Hardy-Weinberg equilibrium. Allele frequencies are presented in TABLE 1.

TABLE 1
ALLELE FREQUENCIES OF OCTN1 AND OCTN2 MUTATIONS

| Group | OCTN1 L503F | OCTN2 G-207C |
|---|---|---|
| healthy controls | F allele: 74 (48%) | C allele: 93 (50%) |
| | I allele: 80 (52%) | G allele: 93 (50%) |
| CD patients from risk | F allele: 42 (78%) | C allele: 37 (77%) |
| haplotype families | I allele: 12 (22%) | G allele: 11 (23%) |
| | p = 0.0003 | p = 0.0014 |
| CD patients without risk | F allele: 1 (0%) | C allele: 1 (3%) |
| haplotype | I allele: 49 (98%) | G allele: 31 (97%) |
| | p < 0.0000001 | p = 0.000002 |

Numbers of chromosomes are shown for each allele, and the significance level for difference from the healthy control group is shown for the two Crohn's Disease patient groups.

Odds ratios for Crohn's Disease susceptibility are presented in TABLE 2.

TABLE 2
RELATIVE RISK FOR CROHN'S DISEASE

| Mutation | One copy | Two copies |
|---|---|---|
| OCTN1 L503F | 5.52 | 18.67 |
| | 95% C.I. (0.66-46.20) | 95% C.I. (2.25-154.92) |
| OCTN2 G-207C | 4.04 | 14.00 |
| | 95% C.I. (0.48-33.88) | 95% C.I. (1.69-115.84) |

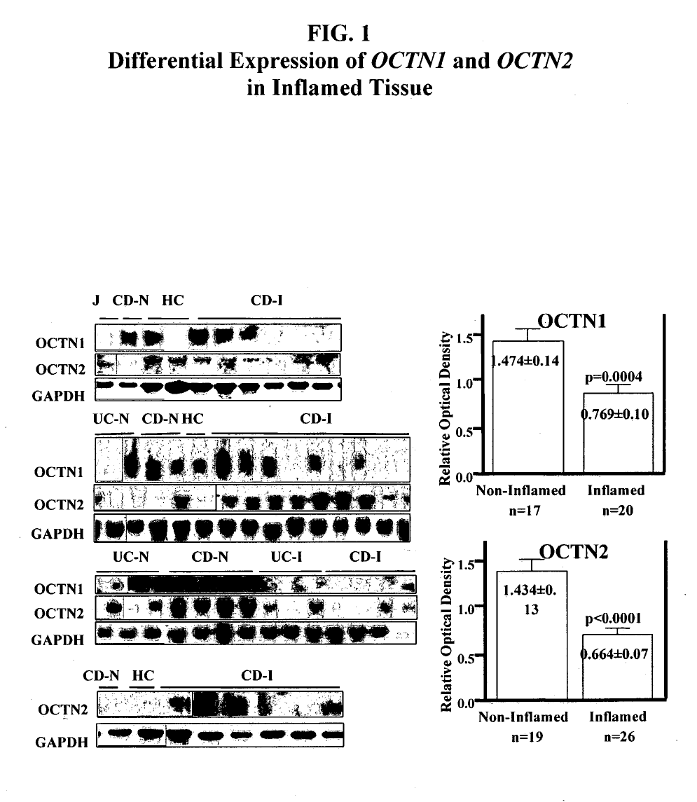

Northern blot analysis. Northern blot analysis of RNA from gastrointestinal tract tissue of Ulcerative Colitis and Crohn's Disease patients revealed a significant downregulation of both OCTN1 and OCTN2 in actively inflamed tissue as compared with uninflamed tissue. To control for variability in expression level between individuals, we also examined matched inflamed and non-inflamed tissue sample pairs from the same patient. The results showed that both OCTN1 and OCTN2 had significant downregulation in inflamed tissue. See, FIG. 1.

Surgical tissue samples were obtained from patients undergoing scheduled surgical resection for either Crohn's Disease or Ulcerative Colitis. Ethics approval and informed consent were obtained prior to sample collection. Samples were preserved in RNAlater (Ambion) prior to RNA extraction. An adjacent section of tissue was used for standard pathological examination to verify the diagnosis (Crohn's Disease, UC), region of the gastrointestinal tract, and status (inflamed, non-inflamed) of tissue. Control colon RNA from unaffected individuals was purchased from Clontech. Total RNA was extracted from tissue using TRIZOL (Gibco BRL). RNA samples (15 μg per lane) were separated in 1% agarose-3.7% formaldehyde denaturing gel, transferred onto MSI nylon transfer membrane (Osmonics Laboratory Products), and cross-linked by ultraviolet irradiation.

The membranes were hybridized with ³²P-labeled complementary probes for OCTN1 and OCTN2. OCTN1 transcripts were detected with a 0.8-kb NotI/EcoRI fragment of OCTN1 (ResGen) containing the 5′ part of the coding region of the human OCTN1 gene. OCTN2 transcripts were detected with 2.2 kb NotI/SalI fragment of OCTN1 (ResGen) containing the 3′ part of the coding region of the human OCTN1 gene. Probes were radiolabeled with [α³²P]-dCTP using random priming (Roche Diagnostics GmbH). The unincorporated radiolabeled nucleotides were removed by ProbeQuant G-50 Micro column (Amersham Pharmacia Biotech). The Northern blots containing total RNA from human tissues were hybridized in QuickHyb hybridization solution (Stratagene, La Jolla, Calif., USA) at 68° C. for 2 hrs. The blots were then washed twice in 2×SSC containing 0.1% SDS at room temperature for 15 min and once at 60° C. for 30 min with 0.1×SSC containing 0.1% SDS.

Densitometry of Northern blots was performed using Scion Imaging Software (Scion Corporation), normalized to G3PDH, and expressed as relative optical density units. Data were analyzed with GraphPad Prism.

Tissue expression analysis. For expression analysis, RT-PCR of multiple tissue expression panels (Clontech) was performed according to the manufacturer's directions. First-strand cDNA preparations from human gastrointestinal (GI) tract and from human immune system, normalized against several housekeeping genes were used to assess tissue specificity and relative abundance of OCTN1 and OCTN2 mRNA. 5 μl (0.2 ng/μl) of each cDNA were used as a template for RT-PCR using gene-specific primers.

For OCTN1, the forward primer was 5′-ACCATCGCCAACTTCTCGGC-3′ (SEQ ID NO:9) and the reverse primer was 5′-CTTTCTGCTGCTTCAGGGGA-3′ (SEQ ID NO:10).

For OCTN2, the forward primer was 5′-CCATCGCCAACTTCTCGG-3′ (SEQ ID NO:11) and the reverse primer was 5′-AATGTTGTGGGACTGCTGCTTC-3′ (SEQ ID NO:12).

After 32 cycles, 5 μl sample of each PCR product was run on 2% agarose/ethidium bromide gel. G3PDH PCR primers and a control cDNA were used as a positive control.

RT-PCR expression analysis of multiple tissue panels from the gastrointestinal (GI) tract and immune system of healthy control individuals demonstrated widespread low levels of expression of OCTN1 and OCTN2, with OCTN1 highest in ascending colon and fetal liver, and OCTN2 highest in colon and placenta. RT-PCR results are summarized in TABLE 3.

TABLE 3
RT-PCR EXPRESSION ANALYSIS OF OCTN1 ANP OCTN2

| Gene | GI tract panel | Immune system panel |
|---|---|---|
| OCTN1 | low, highest in ascending colon | high in fetal liver |
| OCTN2 | low, medium in colon | low, but high in placenta |

Transient expression of OCTN1 (transporter assays) and OCT2 (luciferase promoter assays). For transient expression of wild-type or mutant (L503F; SEQ ID NO:7), HeLa cells were plated out at a density 1.5×10⁶-10⁵ cells/dish in 100-mm plastic culture dishes (Falcon) and carnitine deficient human fibroblasts, (GM 10665) were plated out at a density 2.5×10⁵ cells/well in 6-well plastic culture dishes (Falcon) 24 hr prior transfection with LipofectAMINE PLUS™ Reagent (Invitrogen, La Jolla, Calif., USA) as recommended by the manufacturer.

For HeLa cell culture, human HeLa cells were grown in D-MEM supplemented with 10% fetal bovine serum, 2 mM L-glutamine, 100 U/ml penicillin, and 100 μg/ml streptomycin. For culture of carnitine deficient human fibroblasts from a patient with primary carnitine deficiency, described by Scaglia F. et al., Genet. Med. 1,34-39 (1998) (see description of the patient GM 10665 in National Institute of General Medical Sciences catalog) were obtained from the National Institute of General Medical Sciences Human Genetic Mutant ell Repository, Coriell Cell Repositories (Camden, N.J., USA). Fibroblasts were grown in D-MEM supplemented with 15% fetal bovine serum, 2 mM L-glutamine, 100 U/ml penicillin, and 100 μg/ml streptomycin and 1× concentration of non-essential amino acids. Cells were cultured in 37° C. humidified incubator with a mixture of 5% CO₂ and 95% air.

Forty-eight hr after transfection the media was removed and replaced with Earle's balanced salt solution containing D-glucose (5.5 mM) and supplemented with 0.5% BSA.

For the OCTN1 transporter assays, carnitine transport measurements were made at room temperature using L-[³H]carnitine as substrate, (specific activity 81 Ci/mmol, Moravek Biochemicals Inc.). The transport buffer was composed of 25 mM HEPES/Tris pH 7.5, supplemented with 140 mM NaCl, 5.4 mM KCl, 1.8 mM CaCl₂, 0.8 mM MgSO4, and 5 mM glucose. The transport medium containing the radiolabeled carnitine were preincubated at 37° C. and then added to the cells. After incubation for 30 min at 37° C., transport was terminated by aspiration of the buffer followed by four washes with ice-cold transport buffer. The cells were then solubilized with 1× cell culture lysis reagent (Promega), and the associated radioactivity was quantitated in a liquid scintillation counter. Cellular protein content was determined according to the bicinchoninic acid method of Smith P K et al., Anal. Biochem. 150: 76-85 (1985), using BCA standard protein assay reagent kit (Pierce). To normalize for the transfection efficiency, cells were co-transfected with 20-fold less firefly luciferase plasmid pGL3-P vector. The luciferase activity was measured on manual luminometer (Lumat LB 9501, Berthold). The cells transfected with empty vector under similar conditions served as control. All assays were carried out in triplicate.

For the OCTN2 luciferase promoter assays, a Dual-Luciferase Reporter Assay System (Promega) was used to study the effect of the mutation on the OCTN2 promoter region. The OCTN2-promoter luciferase reporter gene constructs were generated by PCR amplification of approximately 2.7 kb OCTN2-promoter fragments using genomic DNA from a patient homozygous for the G allele as a template and ligation into Bg/II/MluI site of the promoterless luciferase vector pRL-null (Promega) that uses luciferase from Renilla reniformis as a reporter. For PCR primers, two primers, OCTN2-BglII:

5′-GAAGATCTTGGAAGGCTCAGGTGGGAGG-3′ (SEQ ID NO:13) and OCTN2-M1uI: 5′-CGACGCGTGGCAGCAGGCGACCCAAGAC-3′ (SEQ ID NO:14) were used. PCR was performed using PCRx Enhancer System (Invitrogen, La Jolla, Calif., USA) with addition of 1× final PCRx Enhancer solution concentration and with Platinum Taq DNA Polymerase High Fidelity (Invitrogen, La Jolla, Calif., USA), according to a standard protocol with 30 cycles of 94° C. (for 40 sec), 61° C. (for 1 min), and 72° C. (for 3 min) with a 5-min extension at 98° C. at the first cycle and a 10-min extension at 72° C. of the final cycle.

The C mutant construct was then created by replacing the 600 bp fragment encompassing the position −207 by digesting it with EcoNI/NdeI, followed by ligation of the corresponding fragment amplified from a patient homozygous for the C allele.

Constructs were fully sequenced to exclude the possibility of PCR induced errors.

To normalize the activity of the experimental reporter, constructs were cotransfected into HeLa cells with 20-fold less firefly luciferase plasmid pGL3-P as an internal control LipofectAMINE PLUS™ Reagent (Invitrogen, La Jolla, Calif., USA) as per the manufacturer's protocol. 24 hr after transfection, HeLa cells that were undergoing arachidonate treatment were starved for 6 hr in serum-free medium. After starvation, cells were treated at 37° C. with 20 μM arachidonate for 30 min, followed by 2× wash with PBS. Control cells were treated with an equivalent concentration of ethanol. After that the cells were placed in incubator for additional 18 hr in complete media. Cells that were heat shocked 24 hr after transfection were incubated at 42° C. for 2 hr. Cells were then placed back in 37° C.-incubator for additional 18 hr. The luminescence from (Renilla luciferase reaction (“experimental” reporter a:) was measured simultaneously with the activity of firefly luciferase (“control” reporter) using Dual-Luciferase Reporter Assay System (Promega). The instrumentation used was a manual luminometer (Lumat LB 9501, Berthold). The cells transfected with empty vector under similar conditions served as control. All assays were carried out in triplicate.

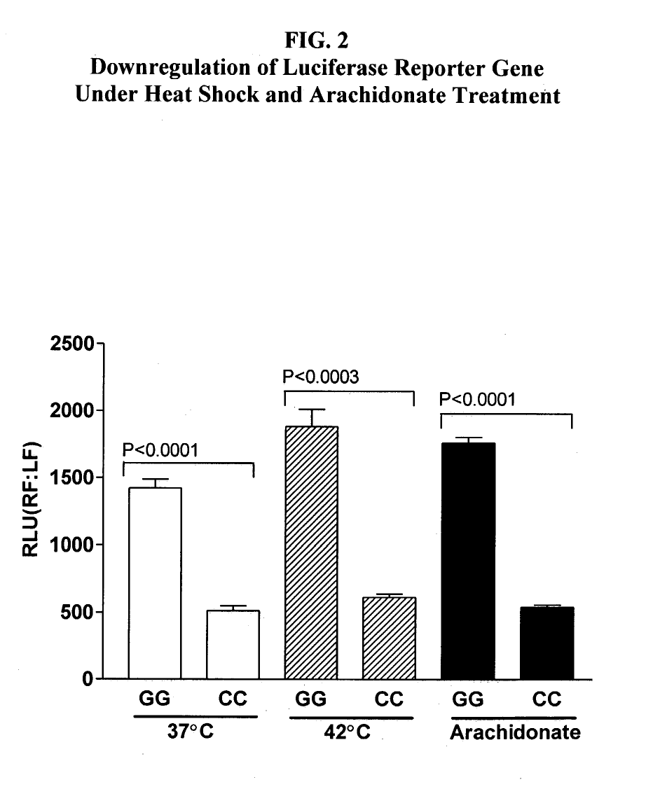

Transfection of the luciferase construct with the C allele of the G-207C mutation of a 2.7 kb fragment of the OCTN2 promoter into HeLa cells resulted in an average 2.8 fold decrease in basal expression as compared with the wild-type (G) allele. Transfection after heat-shock at 42° C. resulted in 3.1 fold reduction of heat shock-induced expression. Transfection after treatment with 20 μM of arachidonic acid at 37° C. also resulted in decreased expression of 3.3 fold as compared to the arachidonate-induced level. Results are presented in FIG. 2.

For OCTN2 gel shift assays, human HeLa cells were grown in D-MEM supplemented with 10% fetal bovine serum, 2 mm L-glutamate, 100 U/ml penicillin, and 100 μg/ml streptomycin at 37° C. in T-75 tissue culture flasks (Sarstedt). Heat-shocked cells were prepared by incubating the flasks in tissue culture incubator at 42° C. for 3 hr. For arachidonate experiment, the day before, the HeLa cells were starved over night with in serum-free D-MEM. The next day cells were washed twice with PBS. Cells were treated then with for 30 min with 20 mM arachidonate at 37° C. The working concentrations were prepared from sodium arachidonate (Sigma) and stored in cold absolute ethanol as a 100 mM stock solution. Control cells were treated with an equivalent concentration of ethanol. Nuclear extracts were prepared by NP-40 based fractionation. This procedure is a modification of the procedure described by Baler R, Dahl G & Voellmy R, Mol. Cell. Biol. 13: 2486-2496 (1993). Briefly, 2.5×10⁶ cells were pelleted, washed twice in 1×PBS and resuspended in 1 ml low salt buffer (A) containing 10 mM Tris HCl, pH 7.5, 10 mM NaCl, 3 mM MgCl₂, 0.5% Nonidet P-40 (NP-40), 0.5 mM phenylmethylsulfonyl fluoride (PMSF). The nuclei were pellet by centrifugation at 7,000 g for 10 min at 4° C. The supernatant was removed and the pellet (the crude nuclei) was suspended in 50 μl high salt buffer (B), containing 20 mM HEPES, pH 7.5, 5 mM MgCl₂, 0.2 mM EDTA, 1 mM dithiothreitol (DTT), 20% glycerol, 300 mM NaCl, 0.5 mM PMSF. After incubation for 15 min at 4° C. the sample was sonicated for 5 sec, centrifuged at 20,000g for 10 min and the resulting extracts was the nuclear fraction. The nuclear extracts were stored at −80° C. The protein concentration of the extracts was measured using the Bradford assay (Bio-Rad).

For gel mobility-shift assays (EMSA), the binding reactions were carried out in a 20 μl volume containing 10 μg of nuclear extract, 0.5 ng ³²P-labeled oligonucleotide probe, 1 μg of poly (dI-dC) and 10 μg of BSA, in 10 mM Tris HCl, pH 7.5, 50 mM NaCl, 1 mM DTT, 1 mM EDTA, 5% glycerol for 2 hr on ice. The antibodies supershift experiments were performed by incubating 2 μl of HSF1 and HSF2 antibodies (TransCruz™ Gel Supershift Antibodies), prior addition of the labeled probes. For the competition experiments a 100-fold molar excess of the unlabeled oligonucleotides was added to each reaction. The competitors, used as positive controls were as follows: WT-OCTN2 (P) Antisense:

5′-CAGGCCCGGAACCTTCCCTGGTCGT-3′ (SEQ ID NO:15); WT-OCTN2 (P) Sense: 5′-ACGACCAGGGAAGGTTCCGGGCCTG-3′ (SEQ ID NO:16); Mutant OCTN2 (P) Antisense: 5′-CAGGCCCGCAACCTTCCCTGGTCGT-3′ (SEQ ID NO:17); and Mutant OCTN2 (P) Sense: 5′-ACGACCAGGGAAGGTTGCGGGCCTG-3′ (SEQ ID NO:18).

The design of the oligonucleotides for the typical HSE for human HSP70 were as follows: Sense 5′-TCGGCTGGAATATTCCCGACCTGGCAGCCGA-3′ (SEQ ID NO:19) and Antisense 5′-TCGGCTGCCAGGTCGGGAATATTCCAGCCGA-3′ (SEQ ID NO:20).

The reaction products were separated electrophoretically on 4% polyacrylamide gel for 3 h at 200 V, dried and exposed to film and detected by autoradiography.

Gel shift assays of the region surrounding the OCTN2 G-207C promoter mutation demonstrated a specific interaction between a nuclear factor and an oligonucleotide containing the region surrounding the wild-type G allele. Binding to this oligo was specific, in that it could be competed by excess wild-type oligo but not by an oligo containing the mutant C allele, nor by a randomly chosen control oligo unrelated to the OCTN2 promoter. Binding of this factor was induced after incubation at 42° C. and arachidonic acid, but not at 37° C., and was identical in mobility to a factor binding to an oligo corresponding to the heat stress element (HSE) from the HSP70 gene. Binding to the G allele of the OCTN2 promoter was also competed by excess HSP70 HSE oligo, further suggesting that the factor was a heat shock transcription factor. Since the G-207C mutation alters a consensus HSF1 binding site, we hypothesized that this factor was HSF1. Anti-HSF1, but not Anti-HSF2, antibody supershifted the complex, positively identifying the unknown factor as HSF1. HSF1 binding to the OCTN2 promoter was completely abolished by the presence of the C allele under all tested conditions.

OCTN1 transporter assays. For OCTN1 transport assays, full length OCTN1 cDNA (2.24 kb) was obtained as a full-length genoscope clone ID CS0DJ 003YB03 (ResGen, Invitrogen, La Jolla, Calif., USA). The cDNA clone was subcloned into EcoRI/NotI sites of the pcDNA3 vector. The construct was fully sequenced and found to contain the leucine allele at position 503 (L503). The construct was used as a template for PCR-based site-directed mutagenesis (QuikChange™, Stratagene, La Jolla, Calif., USA) to create the phenylalanine allele (L503F). Parental DNA strands were removed by DpnI digestion, and the nicked circular DNA was used to transform XL-10 Gold supercompetent cells (Stratagene, La Jolla, Calif., USA).

The mutagenic primers used to prepare the L503F mutant were as follows:

| | | |
|---|---|---|
| 5′-GACTGTCCTGATTGGAATCTTCACCCTTTTTTTCCCTGA-3′ | (forward) | (SEQ ID NO:21) |
| and | | |
| 5′-TCAGGGAAAAAAAGGGTGAAGATTCCAATCAGGACAGTC-3′. | (reverse) | (SEQ ID NO:22) |

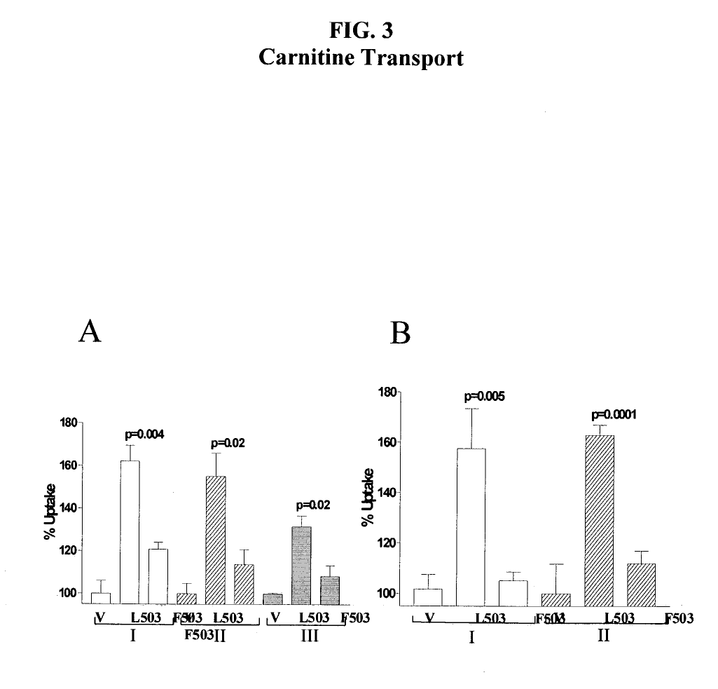

The phenylalanine allele of the L503F mutation resulted in a 3.6 fold reduction toward the vector-alone baseline in uptake of radiolabeled carnitine after transient transfection into HeLa cells (FIG. 3A), as compared with an identical construct containing the leucine allele. In a cell line from an individual lacking OCTN2 expression, the OCTN1 L503F mutation resulted in a 5.2-fold reduction toward vector baseline in uptake of radiolabeled carnitine as compared with the leucine allele (FIG. 3B).

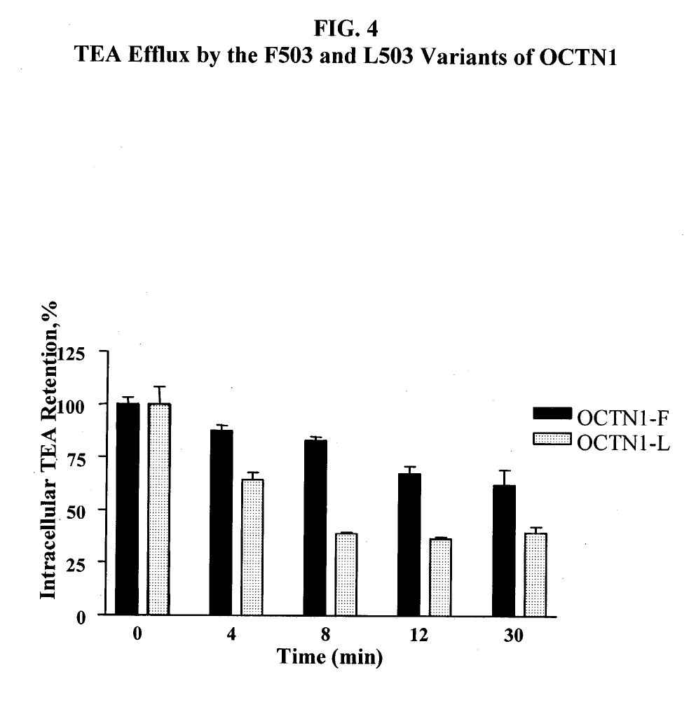

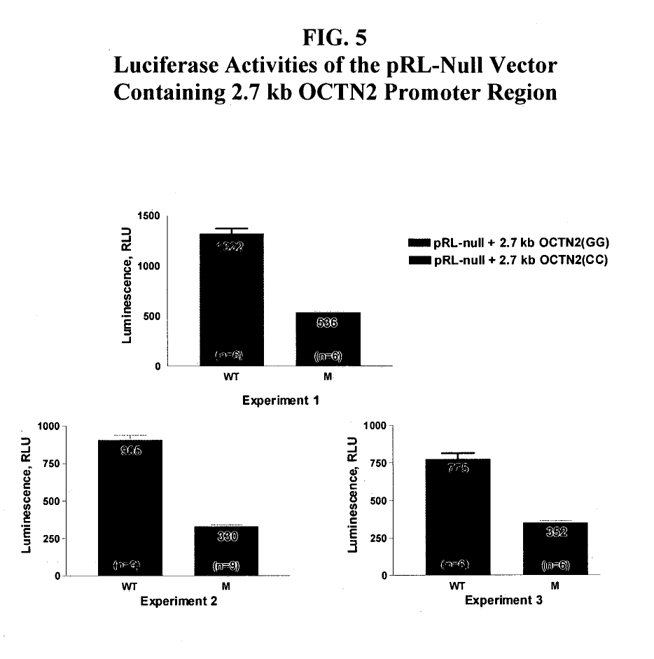

TEA efflux assays: Measurement of efflux of radiolabeled TEA demonstrated that there was a significant decrease in efflux by the CD-associated phenylalanine (L503F) variant of OCTN1 as compared with the leucine variant (L503). See, FIG. 4.

Transfected Phoenix mouse fibroblast cells were loaded with ¹⁴C-labeled TEA for 30 min. at 37° C., washed twice in cold transport buffer, and then resuspended in transport medium. Efflux was initiated at 37° C. and measured at 0, 4, 8, 12 and 30 minutes. Measurements were taken by washing twice in cold transport buffer, solubilizing with 1× cell culture lysis reagent (Promega) and quantifying the 14C retention from 150 μL aliquots. Cellular protein content was determined according to the BCA method. For sodium-free experiments, cells were suspended in medium with sodium replaced isotonically with N-methyl-D-glucamine.

Discussion. The two mutations described here co-segregate with the previously described SNP haplotype, are not found on non-risk haplotypes, and contribute to approximately the same degree of genetic susceptibility to Crohn's Disease (TABLE 2). Apparently, Rioux J D et al., Nature Genet. 29: 223-228 (2001) slightly underestimated the genetic risk (i.e., 2-fold genome relative risk for one SNP haplotype, 6-fold for two), likely because the allele frequency of the risk haplotype was previously estimated from non-transmitted parental chromosomes and not from an independent control set, as we have done here.

The mutation causing the L503F change in OCTN1 has been previously identified as a SNP of unknown function (dbSNP identifier rs1050152), although no allele frequency or functional data was previously available. The L503F mutation is common in the general population. This is likely why the phenylalanine variant (which we show here to be associated with Crohn's Disease susceptibility) was initially reported as the wild-type form of the transporter. Tamai I et al, FEBS Lett. 419, 107-111 (1997). Prior functional assays of the OCTN1 transporter have been performed using the phenylalanine variant, without consideration of other variants at this position, as shown herein.

For wild-type variants, the amino acid corresponding to position 503 of OCTN1 is either leucine, isoleucine, methionine or valine in all other known organic cation transporters from mouse, rat or human. Burckhardt G & Wolff N A, Am. J. Physiol. Renal. Physiol. 278, F853-F866 (2000). A change to a bulky phenylalanine residue might have a structural effect on the protein. This residue is located in transmembrane domain 11 of OCTN1, in a region previously shown to be involved in carnitine transport. Our transporter assay results show that the leucine variant of OCTN1 is significantly better at transporting carnitine than the phenylalanine variant. This result disagrees with previous functional analysis of OCTN1, which used the phenylalanine variant (Tamai I et al., FEBS Lett. 419, 107-111 (1997)) and which suggested that the in vivo function of OCTN1 may actually be as a carnitine transporter rather than simply as a polyspecific cation transporter.

In respect of OCTN2, these data shows that basal transcription is downregulated by the C allele of G-207C, and that this allele disrupts the ability of OCTN2 to be upregulated in response to heat shock or arachidonic acid as a result of impaired HSF1 binding and subsequent transcriptional activation.

The mechanism by which these organic cation transporters contribute to the pathology of Crohn's Disease relate to the in vivo metabolic importance of carnitine. Carnitine facilitates transport of long chain fatty acids across the mitochondrial inner membrane for subsequent β-oxidation, and is also important in the maintenance of cellular CoA levels. Carnitine uptake into lymphocytes, along with a corresponding decrease in plasma levels, is a physiological response to inflammation. Symptoms of the related condition Ulcerative Colitis may be due to an energy deficiency in colonic epithelium secondary to poor mitochondrial function due to decreased long-chain fatty acid transport to the mitochondria. Roediger W E, Lancet 2, 712-715 (1980). The haplotype of mutations in Crohn's Disease patients provided by this invention might affect cellular metabolic energy levels in inflamed tissue by combining impaired OCTN1 transporter function with downregulation and inability to respond to heat or inflammatory stress by the OCTN2 gene.

Heat shock proteins and arachidonic acids are involved in response to a variety of cellular stresses, including sepsis, metabolic stress and ischaemia. The heat shock response modulates inflammation through modulation of NF-κB activation. OCTN2 has not previously been described as a heat stress inducible protein, but from the results of this invention OCTN2 is a heat stress inducible protein. Thus, the OCTN2 gene is normally upregulated in response to inflammation through binding of HSF1 protein to its promoter. This in turn mobilizes carnitine and bolster metabolism in the inflamed tissue. Impaired OCTN1 carnitine transporter activity and lowered OCTN2 expression level results in reduced metabolism and either trigger or worsen cellular stress in areas of inflammation.

The two OCTN transporters may therefore function in the inflammatory pathology of Crohn's Disease. Intriguingly, there are reports that topical irrigation of the colon with propyonil-L-carnitine (PLC) can improve some symptoms of Ulcerative Colitis (Giancaterini A et al., Am. J. Gastroenterol. 96, 2275-2276 (2001)), and PLC also inhibits inflammation in various models of vascular inflammation in rodents. Caruso A et al., Pharmacol. Res. 31, 67-72 (1995); Amico-Roxas M et al., Drugs Exp. Clin. Res. 19, 213-217 (1993). Additionally, OCTN1 is a polyspecific cation transporter, and might have a role in the uptake of drugs used to treat CD from the gut. Mutations such as L503F might therefore serve to worsen the condition by reducing bioavailability of therapeutic compounds, or by decreasing the ability of the cell to remove by efflux toxic compounds such as free radicals, toxic metabolites or bacterial toxins.

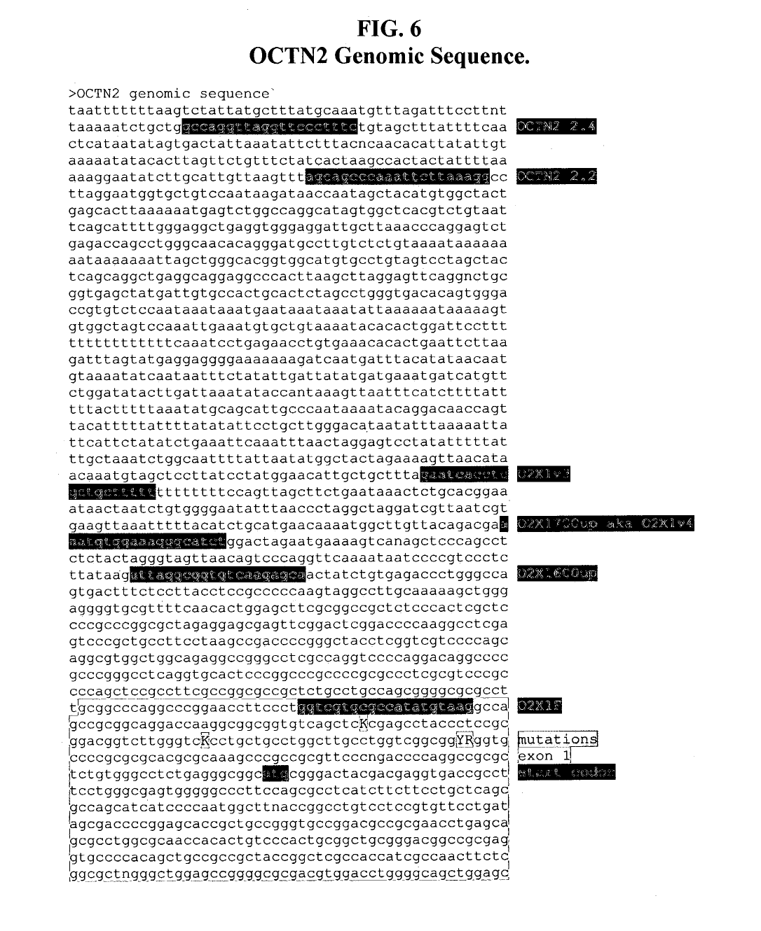

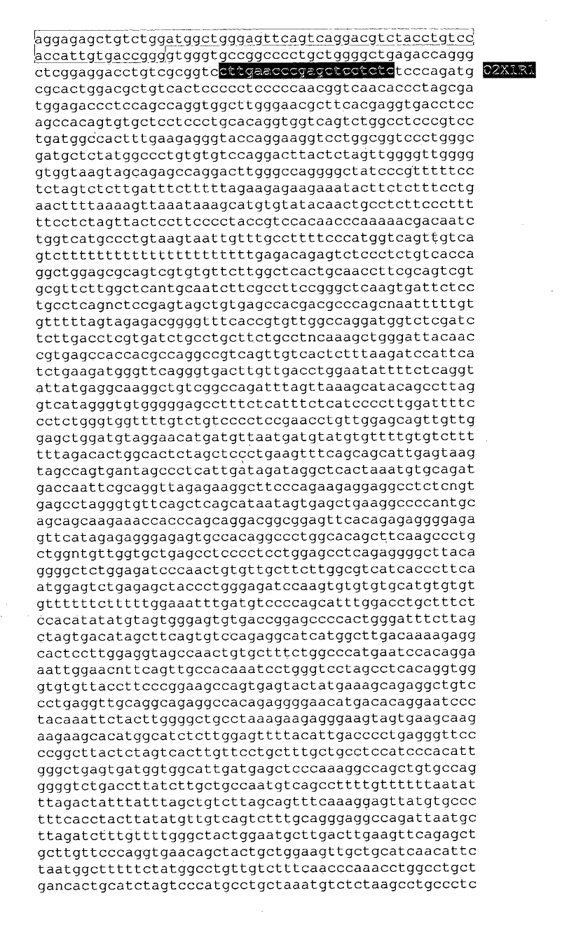

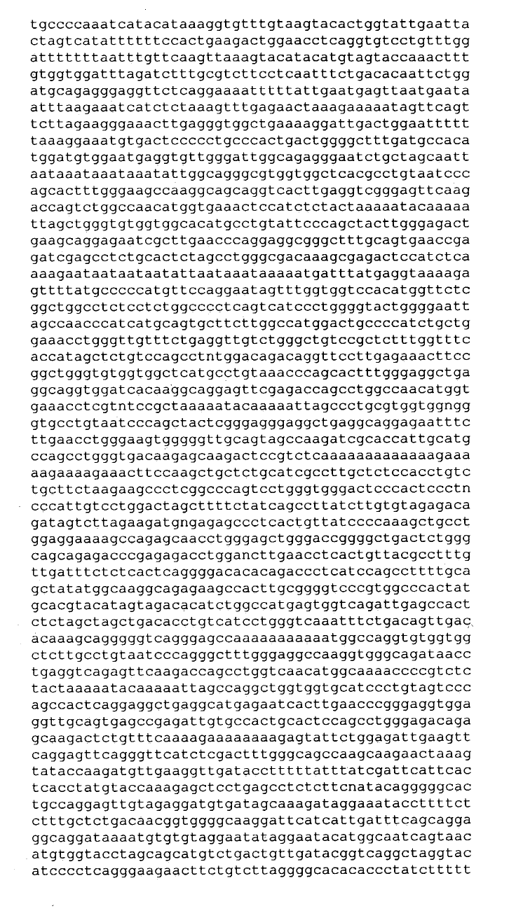

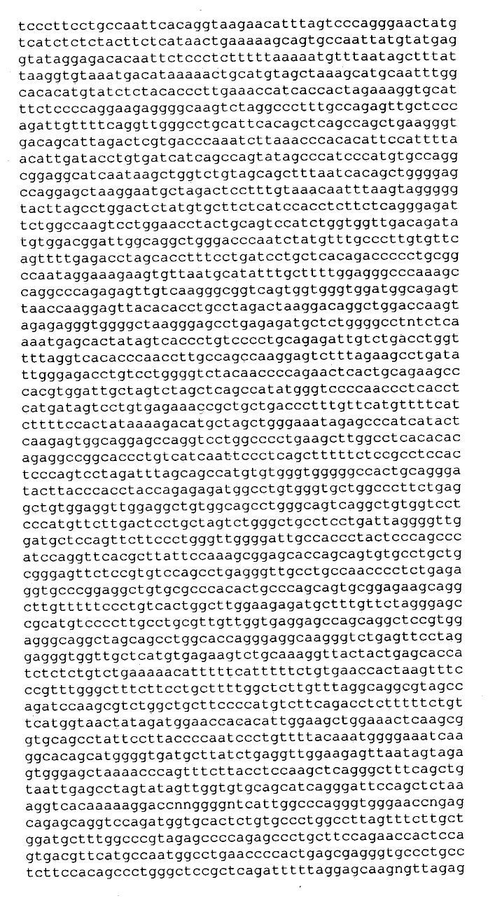

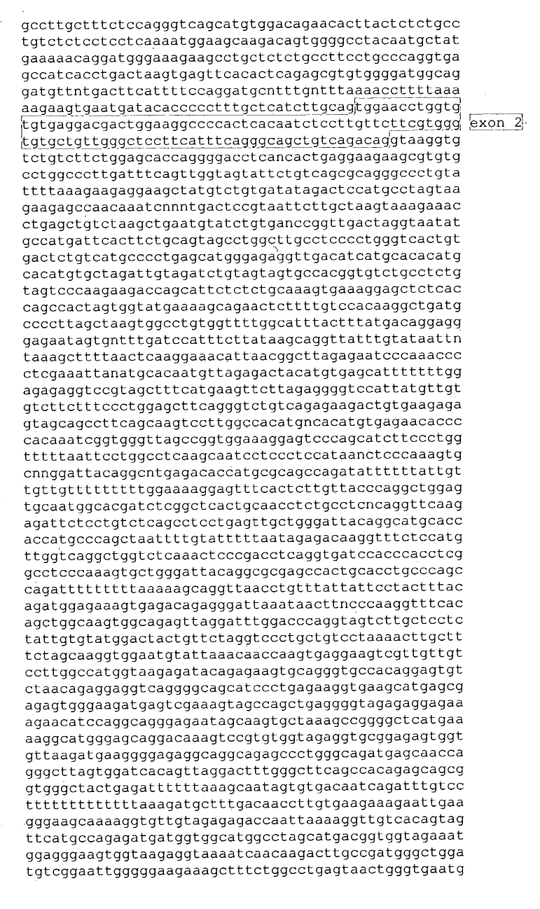

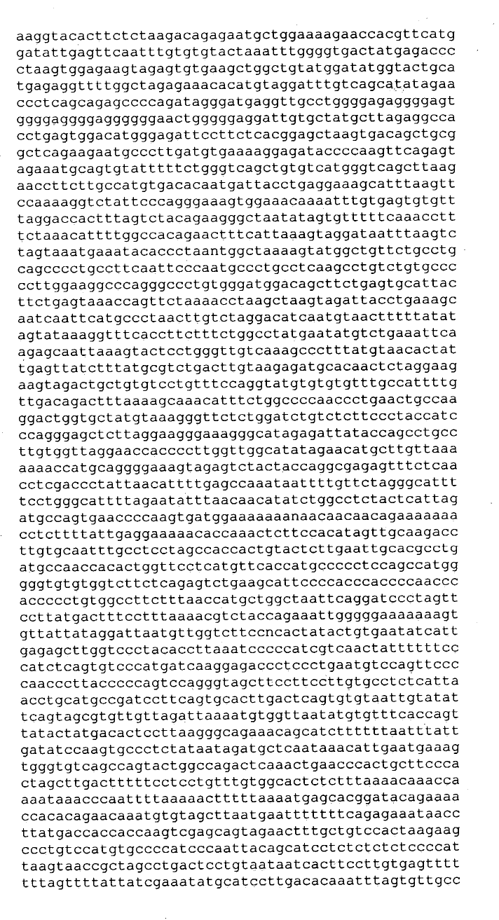

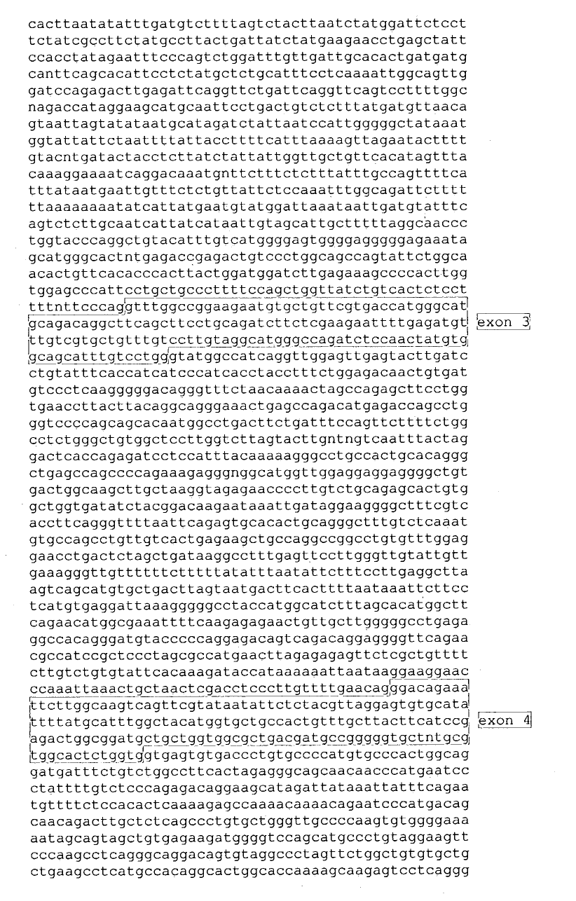

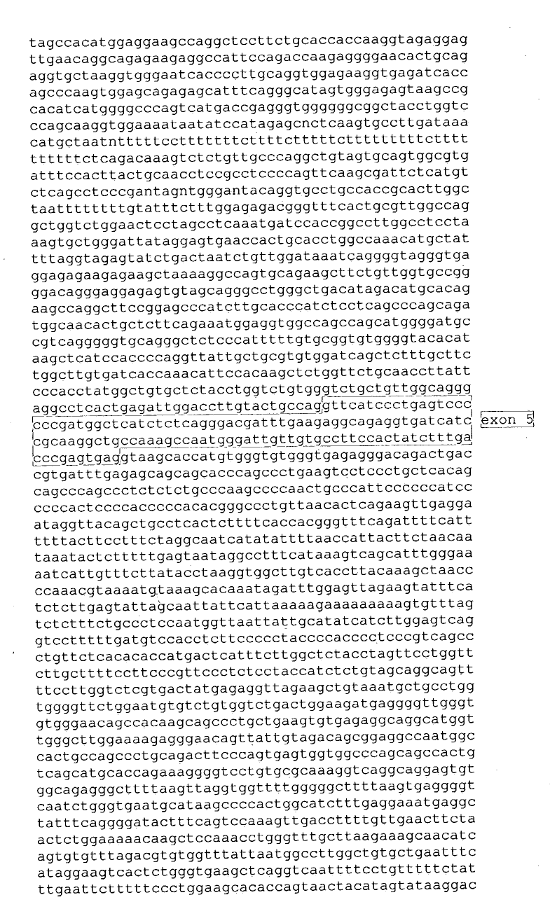

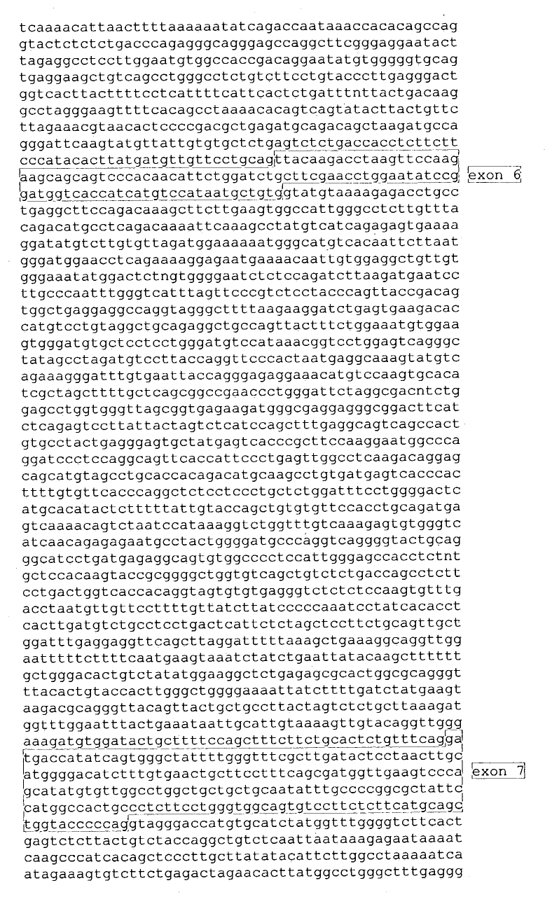

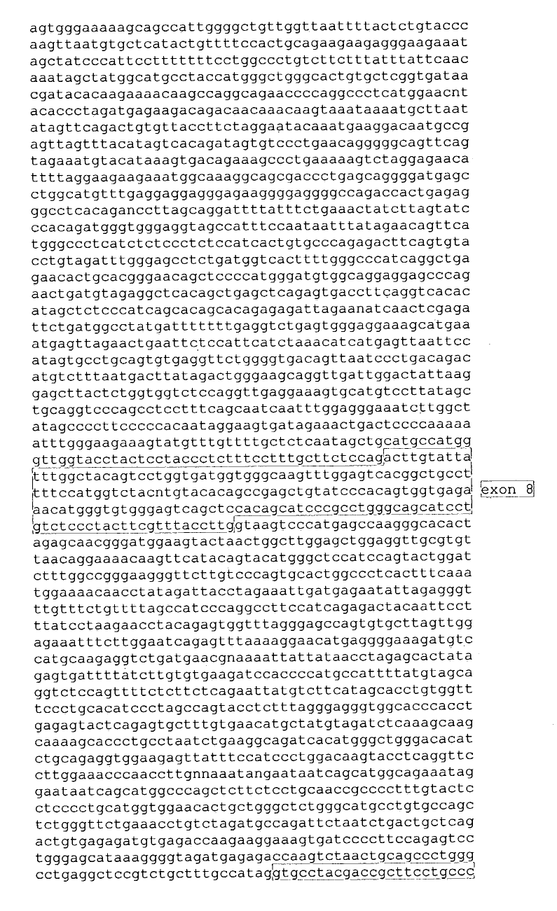

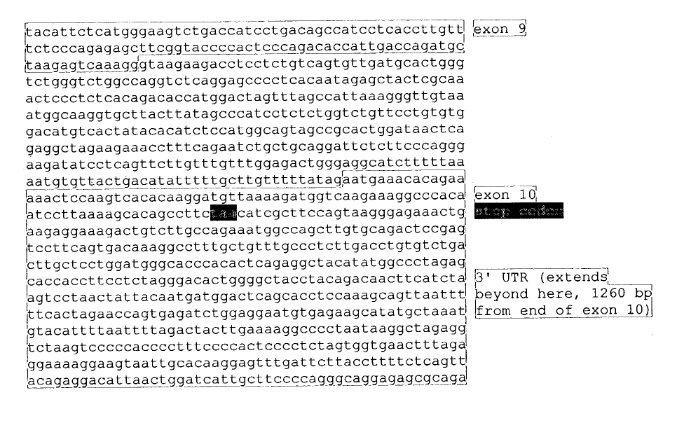

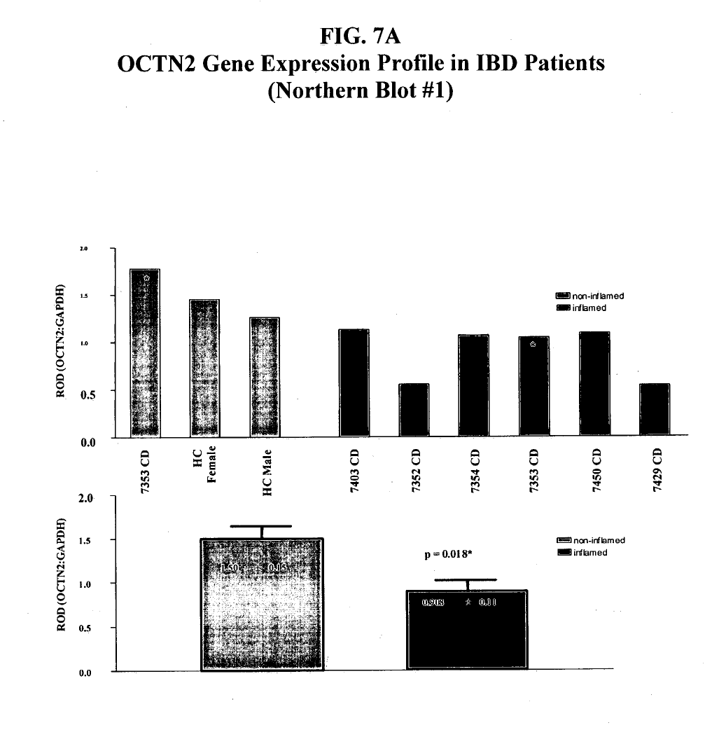

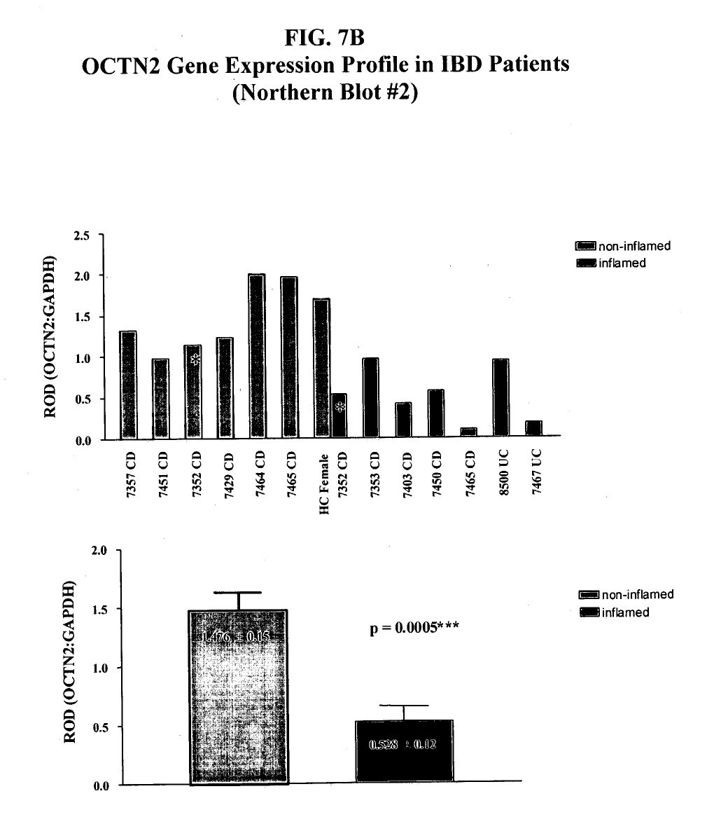

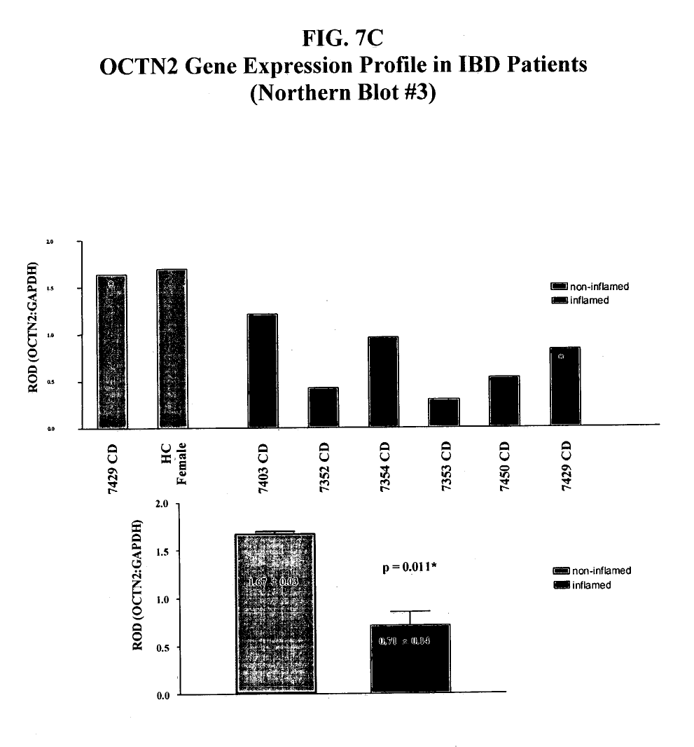

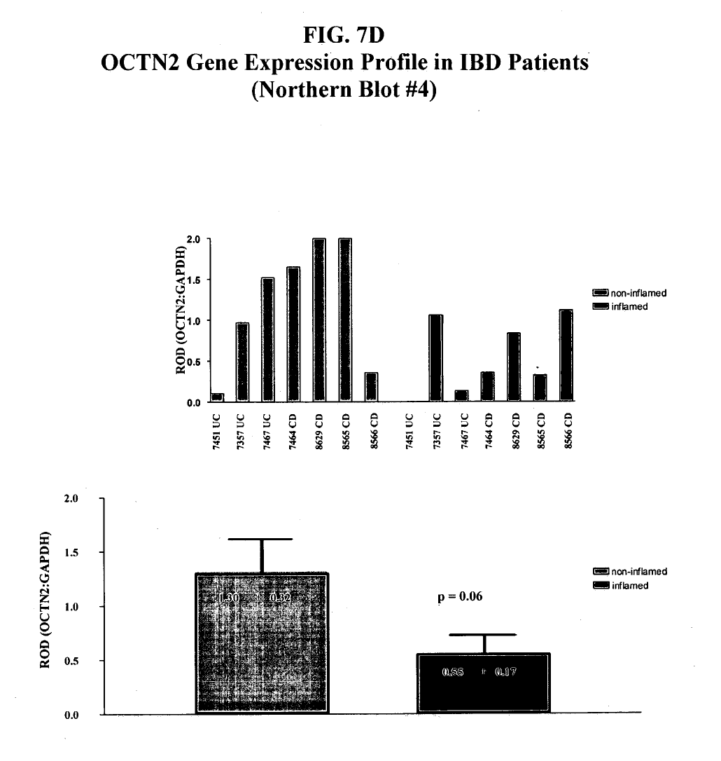

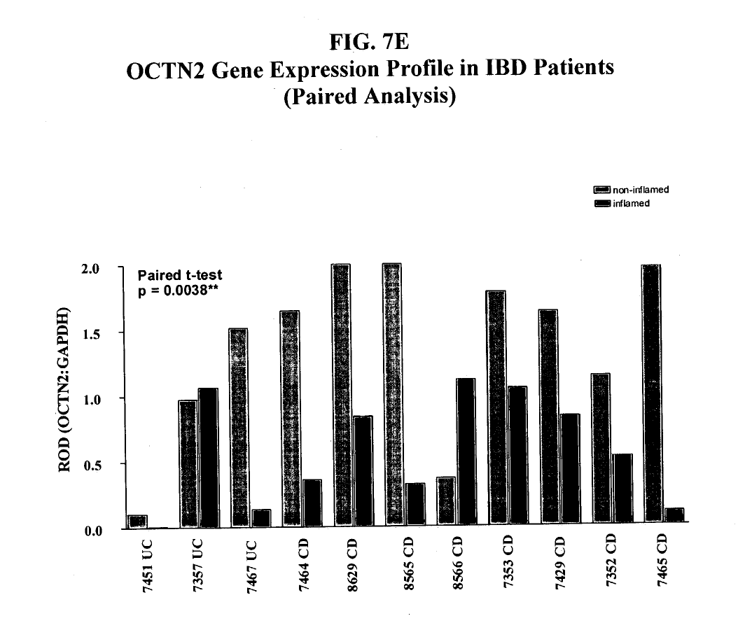

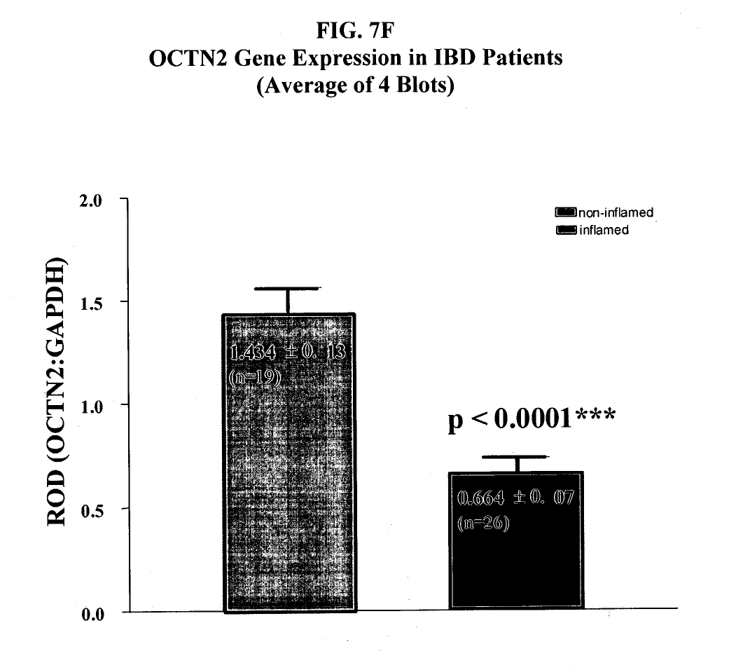

Mutations of the OCTN2 gene and uses in diagnosing inflammation. As described above, OCTN2 is a transporter protein with the ability to transport carnitine in a sodium-dependent manner. Missense mutations and nonsense mutations in the organic cation transporter OCTN2 had previously been identified in patients with primary Systemic Carnitine Deficiency (SCD; (OMIM 212 140)), an autosomal recessive disorder characterized by progressive cardiomyopathy, skeletal myopathy, hypoglycemia and hyperammonemia. By contrast, the invention provides polynucleotide polymorphisms in the OCTN2 gene upstream from the start codon in exon 1 (see, FIG. 6), which are shown here to be related to inflammatory bowel diseases (IBDs), Crohn's disease (CD) and ulcerative colitis (UC). The G-207C mutation of OCTN2, identified above, although it reduces basal transcription level also, is most relevant to transcription induced by heat stress or inflammation. In combination with the accompanying OCTN1 mutation, this haplotype of mutations results in susceptibility to Crohn's Disease.

The invention provides polynucleotide polymorphisms as shown in the following SEQ. ID. NO:23:

gtcccgctgccttcctaagccgaScccgggctacctcggtcgtccccagcaggcgtggctggcagaggccgggcctcgccaggtcccc
aggacaggccccgcccgggcctcaggtgcactcccggcccgccccgcgccctcgcgtcccgccccagctccgccttcgccggcgccg
ctctgcctgccagcggggcgcgccttgcggcccaggcccgSaaccttccctggtcgtgcgccatatgtaaggccagccgcggcaggac
caaggcggcggtgtcagctcKcgagcctaccctccgcggacggtcttgggtcKcctgctgcctggcttgcctggtcggcggYRggtg
ccccgcgcgcacgcgcaaagcccgccgcgttcccngaccccaggccgcgc.

The invention also provides other polynucleotide having sequences that are fragments of SEQ ID NO:23. For example, the fragments can have sequences that that are 20 bases long (or multiples thereof).

Moreover, the invention provides uses for polynucleotides containing the 410 mutation (for example, taagccga(c/g)cccgggcta, SEQ ID NO:24). Alternatively, the invention provides uses for polynucleotides containing the G-207C mutation (for example, ccaggccccg(c/g)aacttccc, SEQ ID NO:25). Other polynucleotides provided by the invention include cggcggtgtcagctc(t/g)cgagcctaccctccgc (SEQ ID NO:26); ggacggtcttgggtc(t/g)cctgctgcctggcttg (SEQ ID NO:27); and cctggtcggcgg(cg/ta)ggtgccccgcgcgcacgc (“TA”; SEQ ID NO:28). Other polynucleotides can be determined from observation of FIG. 6.

OCTN2 Promoter Region. TABLE 2 shows the allele frequencies of the OCTN2 G-207C promoter region mutation. Data on G-207C polymorphism allele frequencies had not previously been obtained in any populations (healthy or diseased).

Alleles are listed in TABLE 4 by number (1 or 2) and the DNA nucleotide present at that position (C or G). The number of chromosomes carrying each allele is shown for either unrelated healthy control individuals (“healthy” column) or Crohn's Disease patients from families with genetic risk conferred by the chromosome 5 locus previously described (“CD” column). The frequency of each allele as a percentage of the total is shown in brackets, and the total number of chromosomes (“n”) is shown at the bottom of each column. The results of a 2 by 2 contingency table chi-squared test are presented at the bottom of TABLE 4, demonstrating a significant difference in allele frequency between the “healthy” and “CD” classes (chi-squared statistic of 10.2640, p value with one degree of freedom 0.0014).

TABLE 4
ALLELE FREQUENCY DATA FOR OCTN2 G-207C MUTATION

| allele | healthy | CD |
|---|---|---|
| 1 (C) | 93 (50%) | 37 (77%) |
| 2 (G) | 93 (50%) | 11 (23%) |
| | n = 186 | n = 48 |

chi-squared = 10.2640
p (1df) = 0.0014

TABLE 5 shows allele frequency data for three polymorphisms of the OCTN2 gene (specifically, the 410, G-207C and “TA” polymorphisms). Allele frequencies are expressed as the number of chromosomes carrying a specific allele followed by the percentage of total chromosomes (“n”) carrying that allele (e.g., for the G-207C mutation in healthy controls, 52 of 110 chromosomes (i.e., 47%) carry the “C” allele). The populations studied are as follows: healthy controls (“healthy”): DNA samples from venous blood of individuals with no history of IBD; RA sibs (“RA”): DNA samples from venous blood of healthy siblings of patients with rheumatoid arthritis but no history of IBD; Wegener's Granulomatosis (“WG”): DNA samples from venous blood of patients with Wegener's Granulomatosis but no history of IBD; surgical cancer/polyp (“cancer”): DNA samples from venous blood of patients undergoing surgery for either colon cancer or colonic polyps but no history of IBD; parents of chr(5) patients: DNA samples from venous blood or cultured white blood cells of parents of CD patients from families showing linkage to chromosome 5; unrelated chr(5) Crohn's Disease (CD) patients (“chr(5) CD”): DNA samples from venous blood or cultured white blood cells of unrelated patients from families showing linkage to chromosome 5; surgical CD: DNA samples from venous blood of patients undergoing surgical removal of bowel tissue as a result of CD; and surgical UC: DNA samples from venous blood of patients undergoing surgical removal of colon tissue as a result of Ulcerative Colitis (UC).

TABLE 5
ALLELE FREQUENCIES OF OCTN2 POLYMORPHISMS
IN VARIOUS POPULATIONS

| | | CONTROL SAMPLES | | | |
|---|---|---|---|---|---|
| | | | | Wegener's | surgical cancer/ |
| mutation | allele | healthy controls | RA sibs | Granulomatosis | polyp |
| G-207C | 1 (C) | 52 (47%) | 8 (44%) | 19 (41%) | 14 (54%) |
| | 2 (G) | 58 (53%) | 10 (56%) | 27 (59%) | 12 (46%) |
| | | n = 110 | n = 18 | n = 46 | n = 26 |
| 410 | 1 (C) | 34 (31%) | 3 (21%) | 12 (33%) | 5 (23%) |
| | 2 (G) | 76 (69%) | 11 (79%) | 24 (67%) | 17 (77%) |
| | | n = 110 | n = 14 | n = 36 | n = 22 |
| TA | 1 (CG) | 105 (94%) | 18 (100)% | 42 (96%) | 19 (79%) |
| | 2 (TA) | 7 (6%) | 0 (0%) | 2 (4%) | 5 (21%) |
| | | n = 112 | n = 18 | n = 44 | n = 24 |
| | | CD FAMILY | | SURGICAL | |
| | | SAMPLES | | SAMPLES | |
| | | parents of chr(5) | unrelated chr(5) | surgical | surgical |
| mutation | allele | patients | CD patients | CD | UC |
| G-207C | 1 (C) | 27 (56%) | 35 (76%) | 13 (54%) | 13 (41%) |
| | 2 (G) | 21 (44%) | 11 (24%) | 11 (46%) | 19 (59%) |
| | | n = 48 | n = 46 | n = 24 | n = 32 |
| 410 | 1 (C) | 10 (21%) | 7 (29%) | 11 (42%) | 14 (47%) |
| | 2 (G) | 38 (79%) | 17 (71%) | 15 (58%) | 16 (53%) |
| | | n = 48 | n = 24 | n = 26 | n = 30 |
| TA | 1 (CG) | 2 (100%) | 18 (90%) | 15 (94%) | 25 (89%) |
| | 2 (TA) | 0 (0%) | 2 (10%) | 1 (6%) | 3 (11%) |
| | | n = 2 | n = 20 | n = 16 | n = 28 |

TABLE 6 shows statistical chi-squared analysis of allele frequencies in the populations as described in TABLE 5. The number of alleles present in these populations is as in TABLE 5. For each test, results are shown for the 410, G-207C and “TA” polymorphisms. Compared populations are indicated along the top of each 2 by 2 contingency table (e.g. “healthy”, “c5 patients”), individual alleles are listed to the left and the total number of counted alleles for each polymorphism is shown below the table (e.g. 110 for the G-207C polymorphism in the first such table). To the right of each table (“stats” column) are listed the chi-squared value (top) and the associated p value based on the one-tailed chi-squared distribution with one degree of freedom (e.g. chi-squared for the first such table is 9.7806, and the associated p value is 0.0018). Some populations are grouped together for these analyses. Where this has been done the nomenclature of TABLE 5 has been followed except for the following: “all CD” means “chr(5) CD and surgical CD combined”; and “all IBD” means “chr(5) CD and surgical CD and surgical UC combined”.

TABLE 6
STATISTICAL ANALYSES OF ALLELE FREQUENCIES

| healthy controls vs. | | | | | healthy controls vs. | | | | |
|---|---|---|---|---|---|---|---|---|---|
| chr(5) CD patients | | | | | all CD patients | | | | |
| G-207C | | healthy | c5 patients | stats | G-207C | | healthy | all CD | stats |
| | 1 | 52 | 35 | | | 1 | 52 | 48 | |
| | 2 | 58 | 11 | 9.7806 | | 2 | 58 | 22 | 7.0203 |
| | | 110 | 46 | 0.0018 | | | 110 | 70 | 0.0081 |
| 410 | | healthy | c5 patients | | 410 | | healthy | all CD | |
| | 1 | 34 | 7 | | | 1 | 34 | 18 | |
| | 2 | 76 | 17 | 0.0058 | | 2 | 76 | 32 | 0.2072 |
| | | 110 | 24 | 0.9393 | | | 110 | 50 | 0.649 |
| TA | | healthy | c5 patients | | TA | | healthy | all CD | |
| | 1 | 105 | 18 | | | 1 | 105 | 33 | |
| | 2 | 7 | 2 | 0.0200 | | 2 | 7 | 3 | 0.0026 |
| | | 112 | 20 | 0.8875 | | | 112 | 36 | 0.9593 |
| healthy controls vs. | | | | | combined healthy/RA/WG | | | | |
| all IBD patients (CD + UC) | | | | | controls vs. all CD patients | | | | |
| G-207C | | healthy | all IDB | stats | G-207C | | all controls | all CD | stats |
| | 1 | 52 | 61 | | | 1 | 79 | 48 | |
| | 2 | 58 | 41 | 2.8540 | | 2 | 95 | 22 | 9.8280 |
| | | 110 | 102 | 0.0911 | | | 174 | 70 | 0.0017 |
| 410 | | healthy | all IDB | | 410 | | all controls | all CD | |
| | 1 | 34 | 32 | | | 1 | 49 | 18 | |
| | 2 | 76 | 48 | 1.3110 | | 2 | 111 | 32 | 0.2894 |
| | | 110 | 80 | 0.2522 | | | 160 | 50 | 0.5906 |
| TA | | healthy | all IDB | | TA | | all controls | all CD | |
| | 1 | 105 | 58 | | | 1 | 165 | 33 | |
| | 2 | 7 | 6 | 0.2143 | | 2 | 9 | 3 | 0.1220 |
| | | 112 | 64 | 0.6434 | | | 174 | 36 | 0.7269 |
| combined healthy/RA/WG vs. | | | | | all controls healthy/RA/WG/ | | | | |
| all IBD (CD + UC) | | | | | cancer vs. chr(5) CD | | | | |
| G-207C | | healthy | all IDB | stats | G-207C | | all controls | c5 patients | stats |
| | 1 | 79 | 61 | | | 1 | 93 | 35 | |
| | 2 | 95 | 41 | 4.7753 | | 2 | 107 | 11 | 11.9582 |
| | | 174 | 102 | 0.0289 | | | 200 | 46 | 0.0005 |
| 410 | | healthy | all IDB | | 410 | | all controls | c5 patients | |
| | 1 | 49 | 32 | | | 1 | 54 | 7 | |
| | 2 | 111 | 48 | 1.6981 | | 2 | 128 | 17 | 0.0350 |
| | | 160 | 80 | 0.1925 | | | 182 | 24 | 0.8516 |
| TA | | healthy | all IDB | | TA | | all controls | c5 patients | |
| | 1 | 165 | 58 | | | 1 | 184 | 18 | |
| | 2 | 9 | 6 | 1.4290 | | 2 | 14 | 2 | 0.0008 |
| | | 174 | 64 | 0.2319 | | | 198 | 20 | 0.9774 |

The most common OCTN2 DNA sequence variation (mutation) identified is significantly increased in frequency in Crohn's Disease patients. This observation is statistically highly significant (p=0.0014). The risk allele is nearly always found on the Single Nucleotide Polymorphism (SNP) risk haplotype background in these patients, fitting our genetic model of Crohn's Disease susceptibility in this region.

In addition, we have detected an association between the OCTN2 gene and generalized inflammatory responses. We performed a gel shift assay that showed a connection between a mutation in the OCTN2 gene, the specific protein that binds to the promoter region of the OCTN2 gene (HSF1; heat shock transcription factor 1, NCBI UniGene identified Hs.1499), and an HSF1 involvement in inflammation. The specific interaction of the Hsf1 protein with the OCTN2 promoter region is abolished by the presence of the G-207C “C” allele.

No complexes with wild-type or mutant oligos (with the OCTN2 promoter region abolished by the presence of the G-207C “C” allele) were detected (by hybridization with ³²P-labeled probes) in the absence of nuclear extract. No complexes were formed between wild-type oligo and nuclear extract from cells that are not heat shocked. Wild-type oligo formed a complex with nuclear-extract from heat-shocked cells that is specifically competed for by cold (50× excess over ³²P-labeled test oligonucleotide) wild-type oligo and by cold oligo containing a known heat shock enhancer (HSE). This complex was not competed for by cold mutant oligo or cold unrelated oligo. This complex was identical in gel mobility to the complex between labeled oligo containing a known HSE and nuclear extract. This complex is supershifted, i.e. further reduced in mobility, upon forming a higher-order complex with anti-HSF1 antibody, indicating that the protein component of the nuclear extract that binds to the complex is Hsf1. No complexes are seen between labeled mutant oligo and nuclear extract, regardless of the presence of competitor oligo or anti-Hsf1 antibody.

Thus, the OCTN2 G-207C mutation forms specific complexes with the HSF1 protein and that the binding of HSF1 is fully abolished by the presence of the G-207C mutant (“C”) allele.

Thus, the invention provides a method for identifying an anti-inflammatory agent. A complex is formed, in vivo or in vitro, between the heat shock factor 1 (HSF1) protein and a polynucleotide containing a mutation in the OCTN2 promoter region. For example, the mutation can be the G-207C mutation in the OCTN2 promoter region. The complex is contacted with an agent, such as a drug, suspected of dissociating the complex. Detecting the dissociation of the complex identifies the agent as being an agent that is an anti-inflammatory agent.

Primers useful for PCR amplification parts of the OCTN2 gene include OCTN2 2.4: gccaggttaggttccctttc (SEQ ID NO:29); OCTN2 2.2: agcagcccaaattcttaaagg (SEQ ID NO:30); O2X1v3: gaatcacctcgctgcttttt (SEQ ID NO:31); O2X1v4: aaatgtggaaagggcatct (SEQ ID NO:32); O2X1600up: attaggcggtgtcaagagca (SEQ ID NO:33); O2X1F: ggtcgtgcgccatatgtaag (SEQ ID NO:34); and O2X1R1: gagaggagctcgggttcaag (SEQ ID NO:35).

Mutations of the OCTN1 gene and uses in diagnosing inflammation. As described above, OCTN1 is a transporter protein with the ability to transport carnitine in a sodium-dependent manner. OCTN1, which has been cloned and characterized in mouse (553 amino acids) and in human fetal liver (551 amino acids), carries a nucleotide binding site motif. OCTN1 is strongly expressed in adult kidney, trachea, bone marrow, and fetal liver, and several tumor cells, but not in adult human liver. OCTN1 mediates uptake of TEA in a pH-dependent manner.

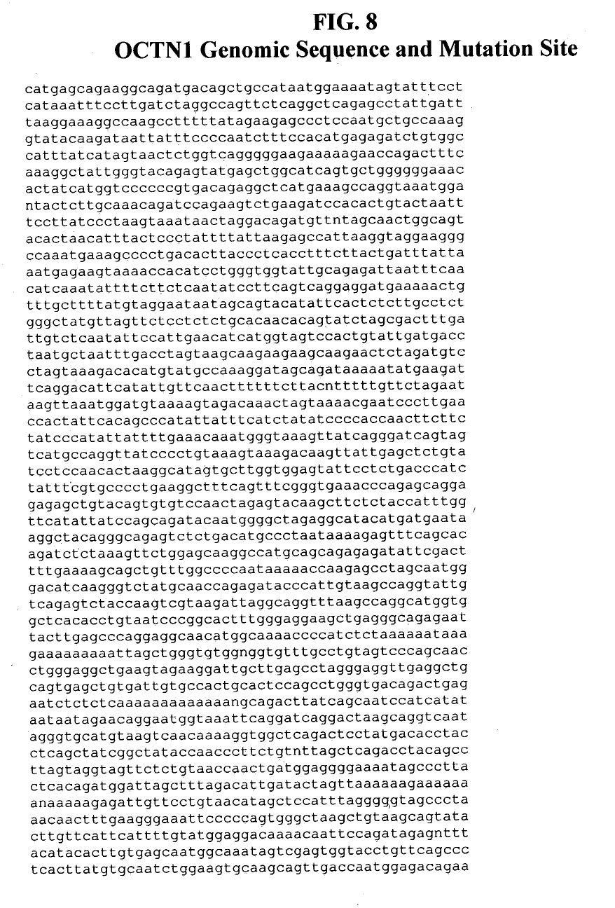

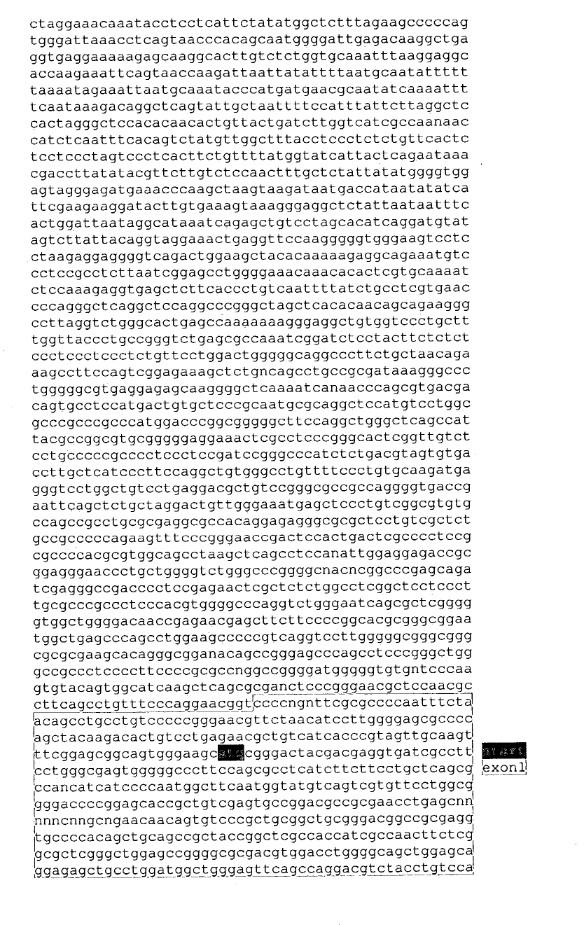

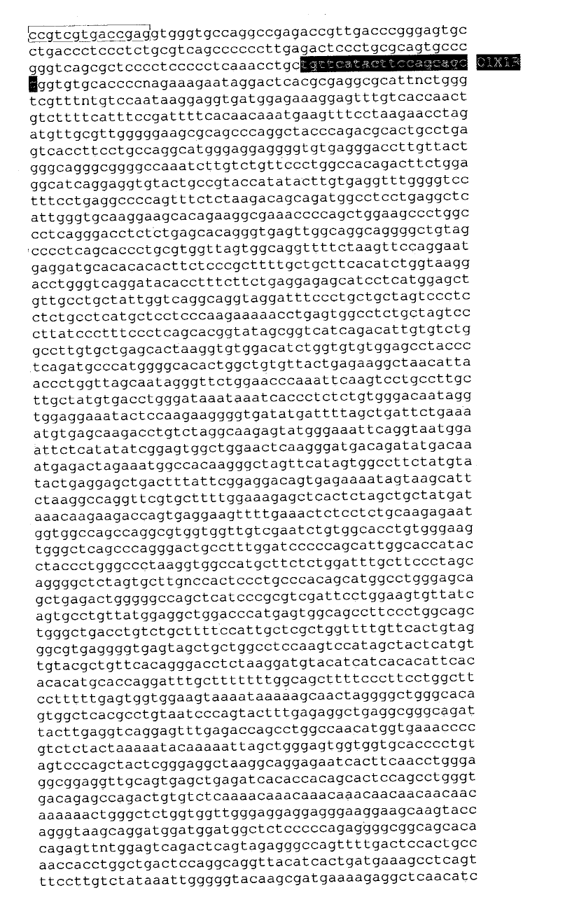

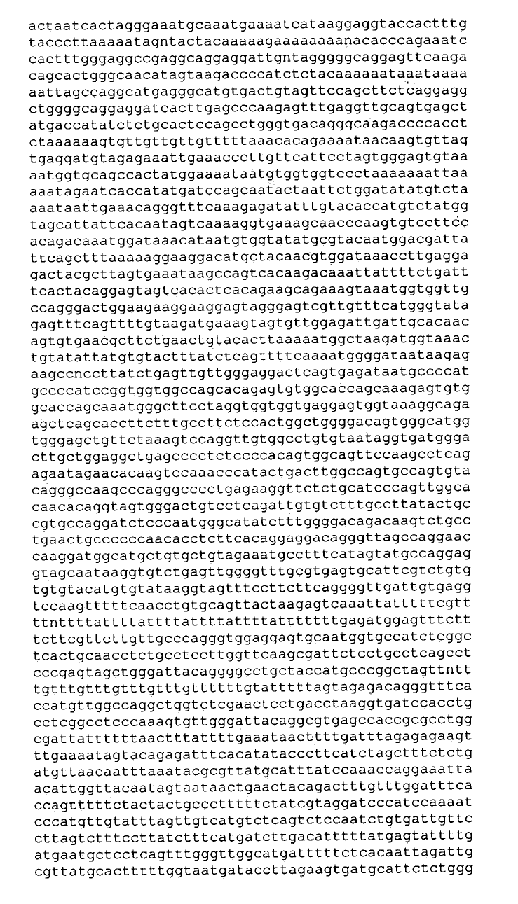

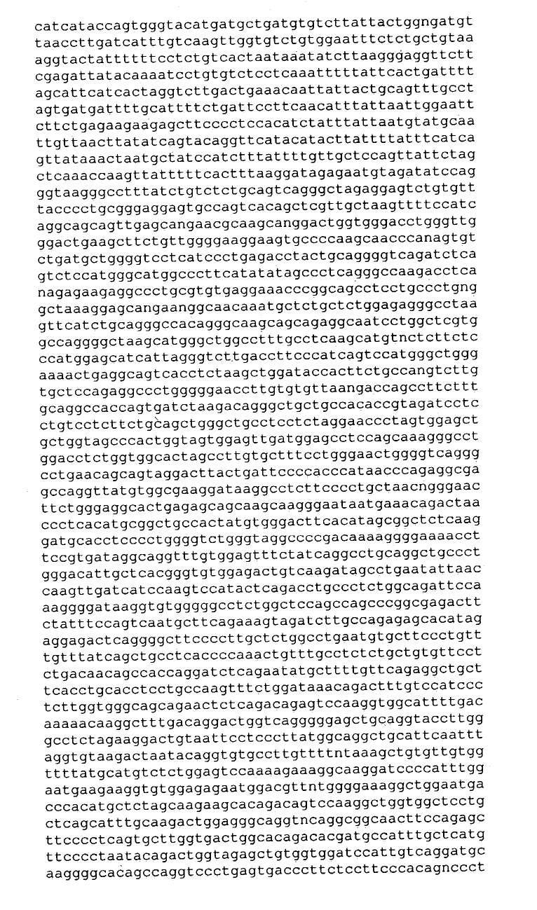

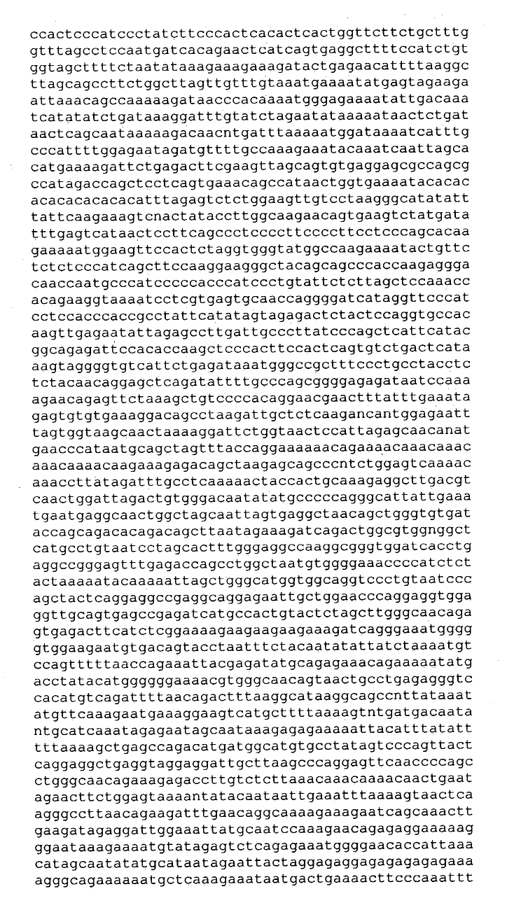

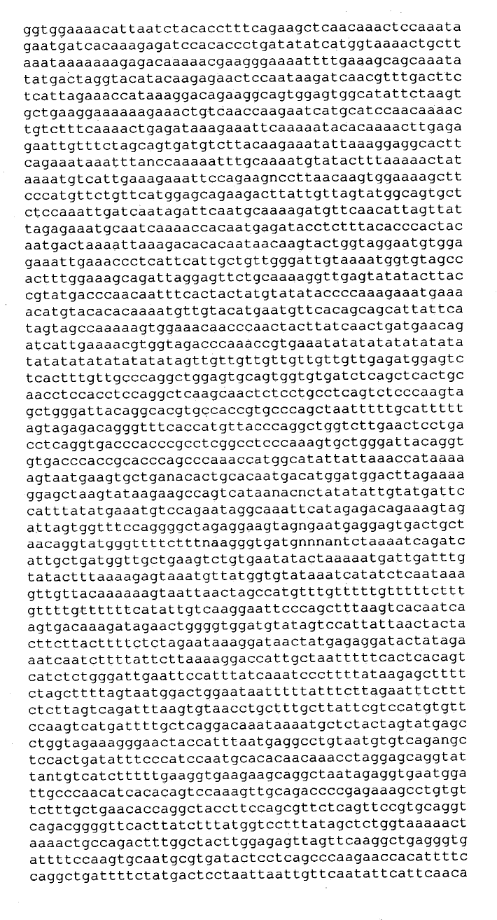

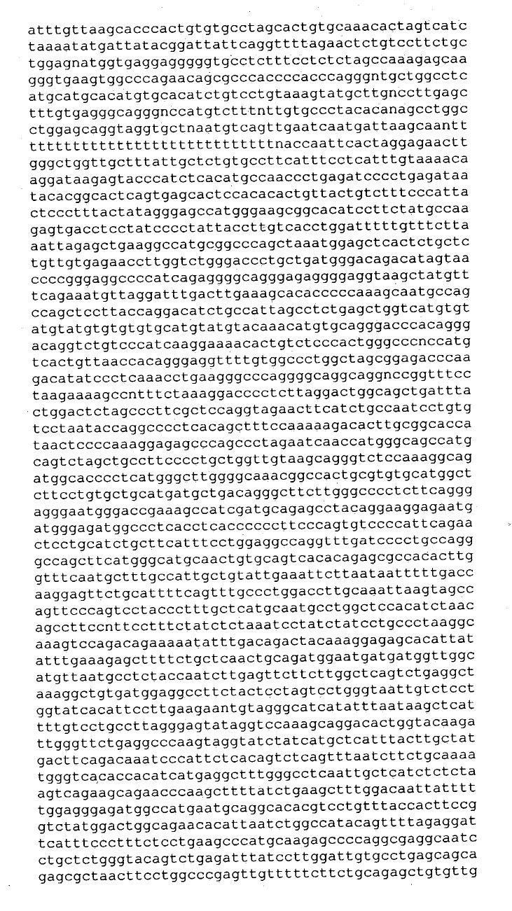

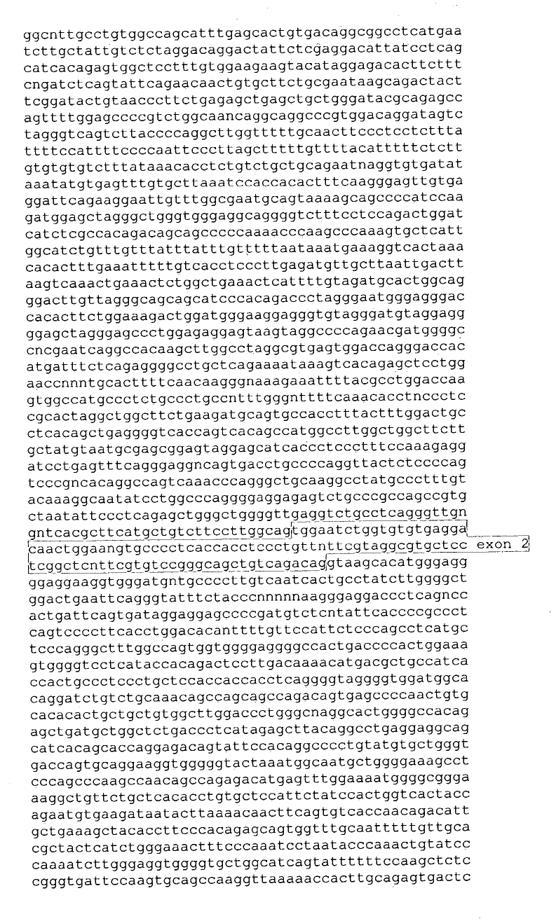

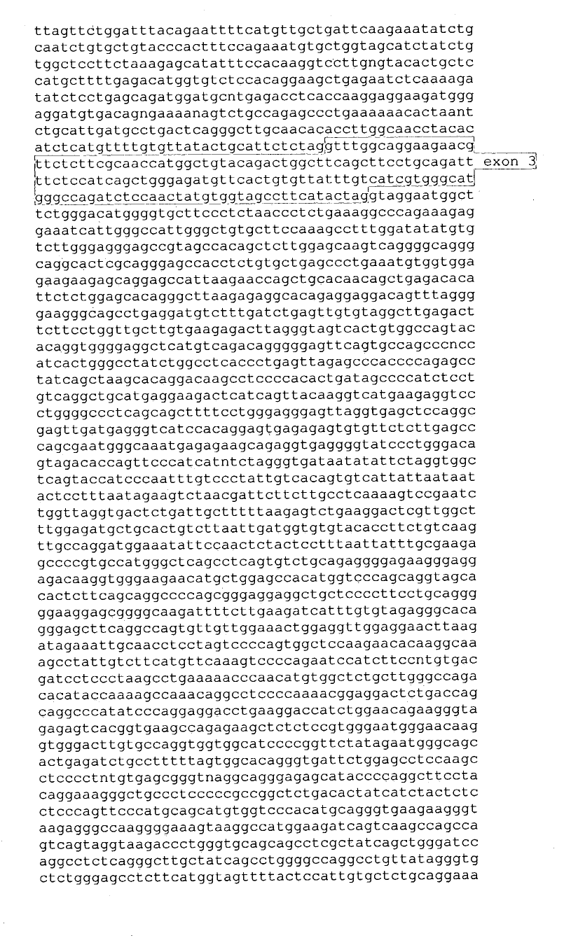

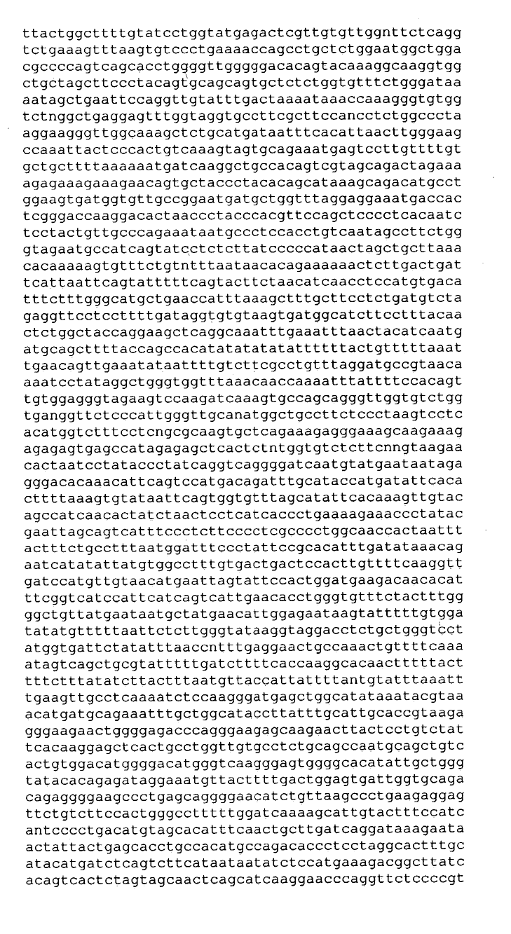

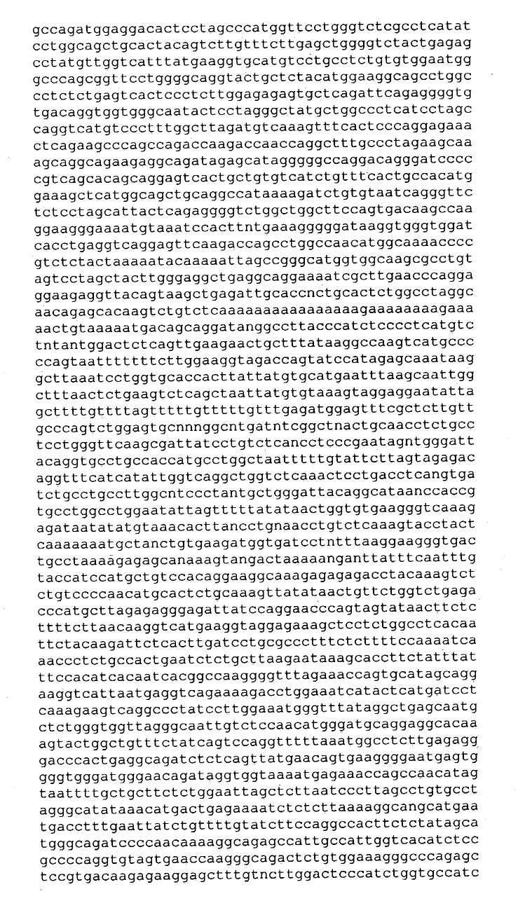

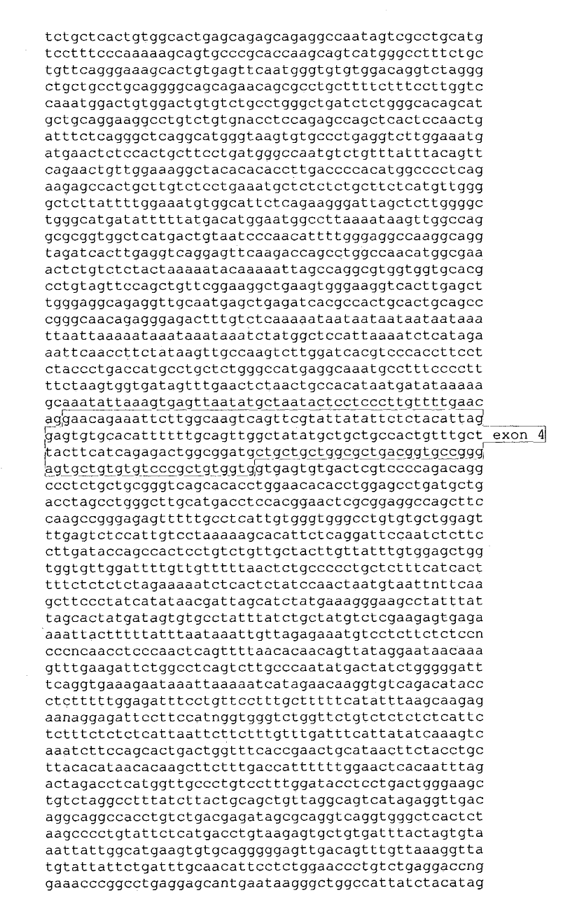

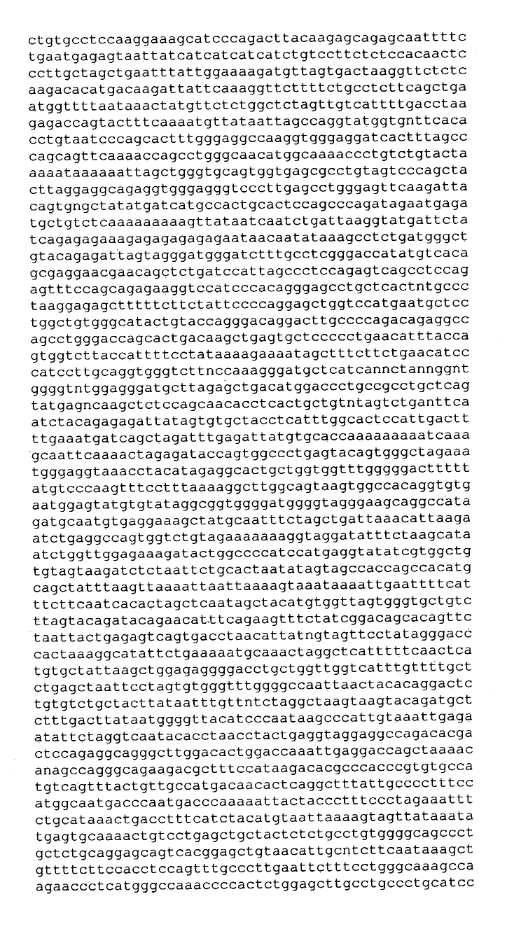

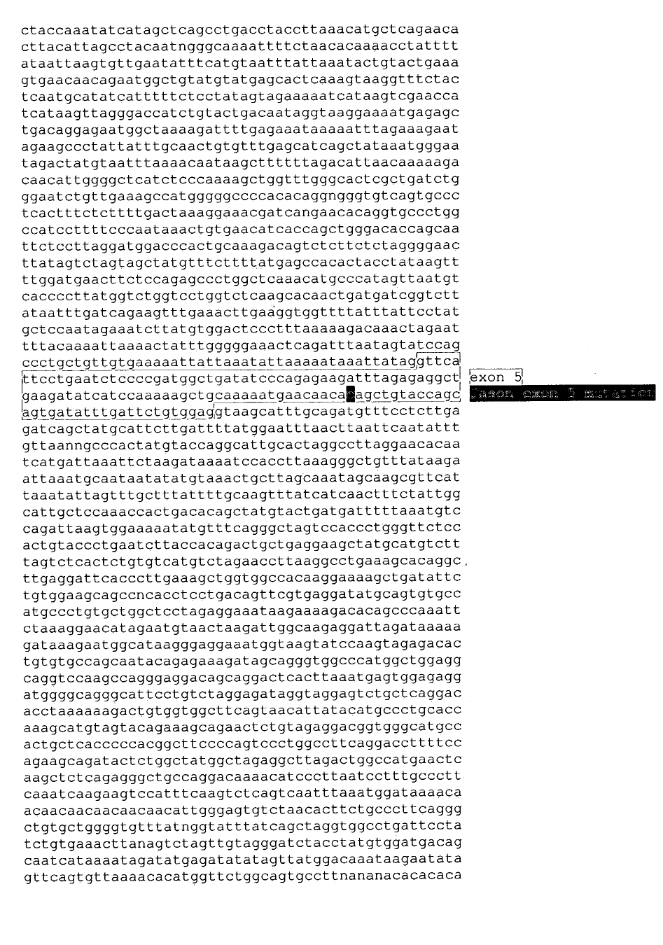

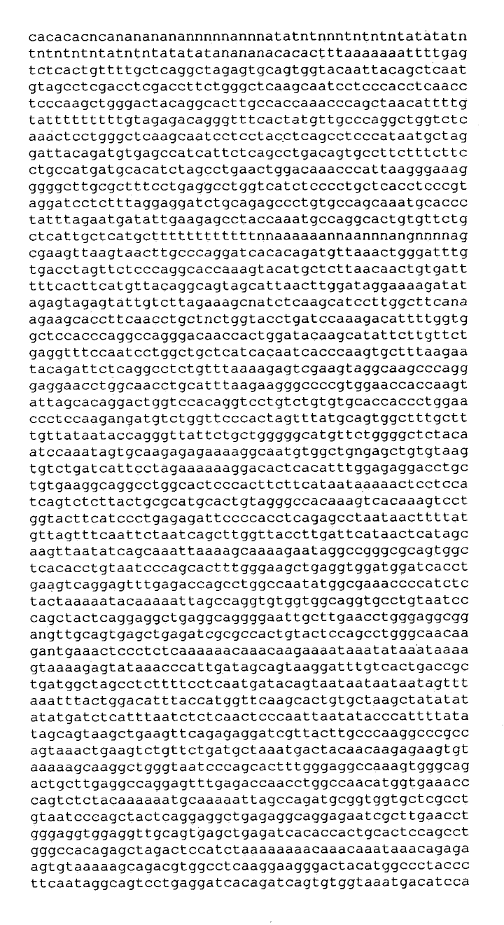

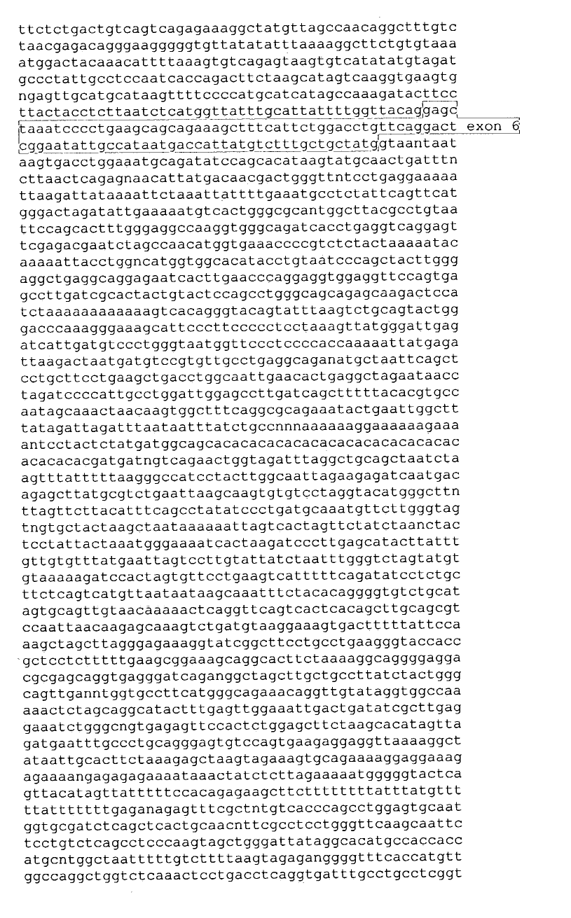

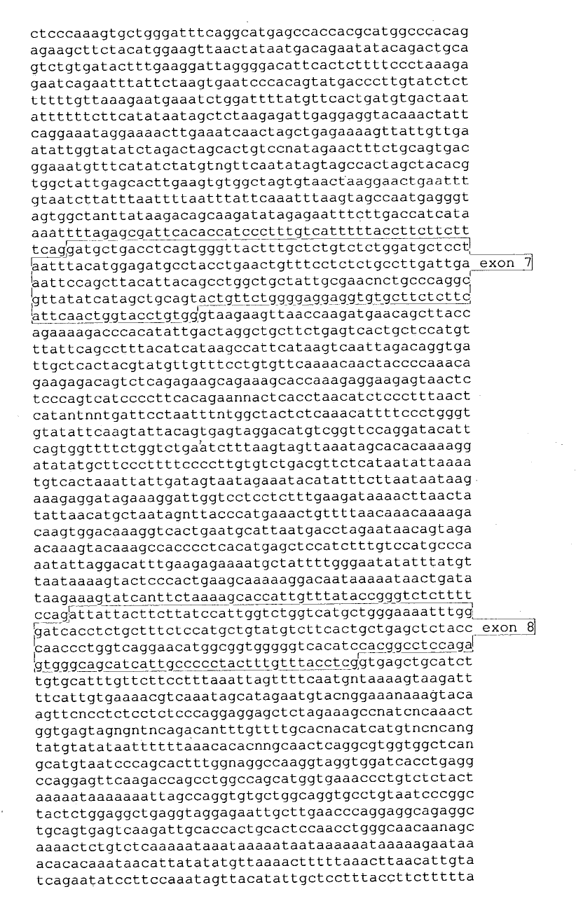

FIG. 8 shows the nucleotide sequence of the OCTN1 gene, including the single nucleotide polymorphism in exon 9. This polymorphism is represented in dbSNP database as rs1050152. The allele frequency of this SNP had not been determined in any population (control or disease related). The nucleotide sequence of exon 9 is provided as: gtgcttacaacagaatgctgccctacatcgtcatgggtagtctgactgtcctgattggaatcYtcaccctttttttccctgaaagtttgggaatga ctcttccagaaaccttagagcagatgcagaaagtgaaatg (SEQ ID NO:36).

The nucleotide sequence of the spliced exons is provided as:

(SEQ ID NO:37)
ccccngnttcgcgccccaatttctaacagcctgcctgtcccccgggaacgttctaacatccttggggagcgccccagctacaagacactgt
cctgagaacgctgtcatcacccgtagttgcaagtttcggagcggcagtgggaagcatgcgggactacgacgaggtgatcgccttcctggg
cgagtgggggcccttccagcgcctcatcttcttcctgctcagcgccancatcatccccaatggcttcaatggtatgtcagtcgtgttcctggcg
gggaccccggagcaccgctgtcgagtgccggacgccgcgaacctgagcnnnnncnngcngaacaacagtgtcccgctgcggctgcg
ggacggccgcgaggtgccccacagctgcagccgctaccggctcgccaccatcgccaacttctcggcgctcgggctggagccggggcg
cgacgtggacctggggcagctggagcaggagagctgcctggatggctgggagttcagccaggacgtctacctgtccaccgtcgtgaccg
agtggaatctggtgtgtgaggacaactggaangtgcccctcaccacctccctgttnttcgtaggcgtgctcctcggctcnttcgtgtccgggc
agctgtcagacaggtttggcaggaagaacgttctcttcgcaaccatggctgtacagactggcttcagcttcctgcagattttctccatcagctg
ggagatgttcactgtgttatttgtcatcgtgggcatgggccagatctccaactatgtggtagccttcatactaggaacagaaattcttggcaagt
cagttcgtattatattctctacattaggagtgtgcacattttttgcagttggctatatgctgctgccactgtttgcttacttcatcagagactggcgg
atgctgctgctggcgctgacggtgccgggagtgctgtgtgtcccgctgtggtggttcattcctgaatctccccgatggctgatatcccagaga
agatttagagaggctgaagatatcatccaaaaagctgcaaaaatgaacaacanagctgtaccagcagtgatatttgattctgtggaggagct
aaatcccctgaagcagcagaaagctttcattctggacctgttcaggactcggaatattgccataatgaccattatgtctttgctgctatggatgct
gacctcagtgggttactttgctctgtctctggatgctcctaatttacatggagatgcctacctgaactgtttcctctctgccttgattgaaattccag
cttacattacagcctggctgctattgcgaacnctgcccaggcgttatatcatagctgcagtactgttctggggaggaggtgtgcttctcttcatt
caactggtacctgtggattattacttcttatccattggtctggtcatgctgggaaaatttgggatcacctctgctttctccatgctgtatgtcttcact
gctgagctctacccaaccctggtcaggaacatggcggtgggggtcacatccacggcctccagagtgggcagcatcattgccccctactttg
tttacctcggtgcttacaacagaatgctgccctacatcgtcatgggtagtctgactgtcctgattggaatcYtcaccctttttttccctgaaagttt
gggaatgactcttccagaaaccttagagcagatgcagaaagtgaaatggttcagatctgggaaaaaaacaagagactcaatggagacana
agaaaatcccaaggttctaataactgcattctgaaaaaatatctaccccatttggtgaagtgaaaaacacaaaaataagaccctgtggagaaa
ttcgttgttcccactgaaatggactgactgtaacgattgacaccaaaatgaaccttgctatcaagaaatgctcgtcatacagtaaactctggatg
attcttccagataatgtccttgctttacaaaccaaccatttctagagagtctccttactcattaattcaatgaaatggattggtaagatgtcttgaaa
acatgttagtcaaggactggtaaaatacatataaagattaacactcatttnccaatcatacaaatactatccaaataaaaat.

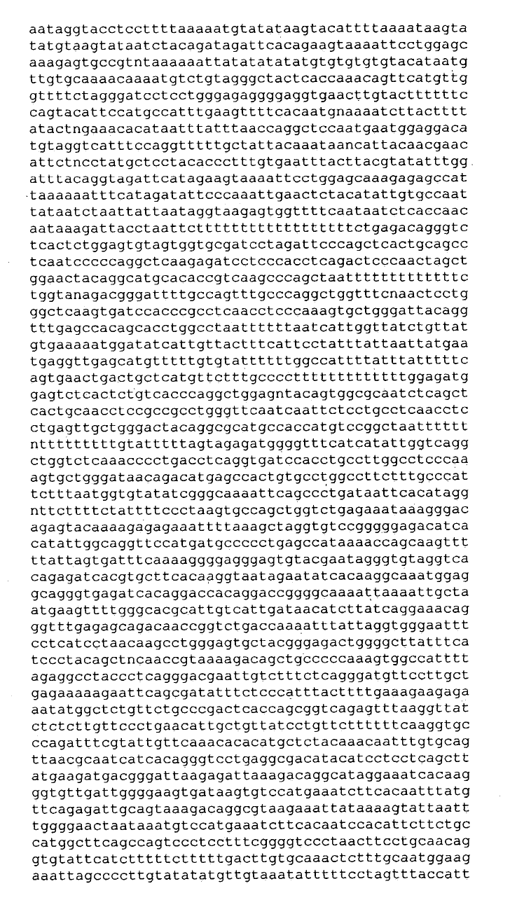

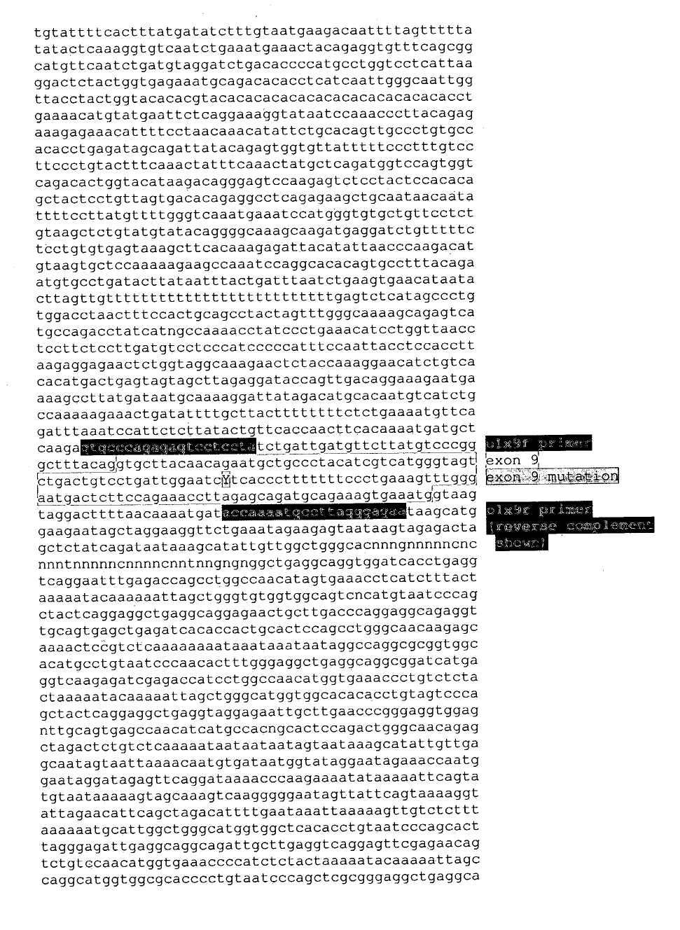

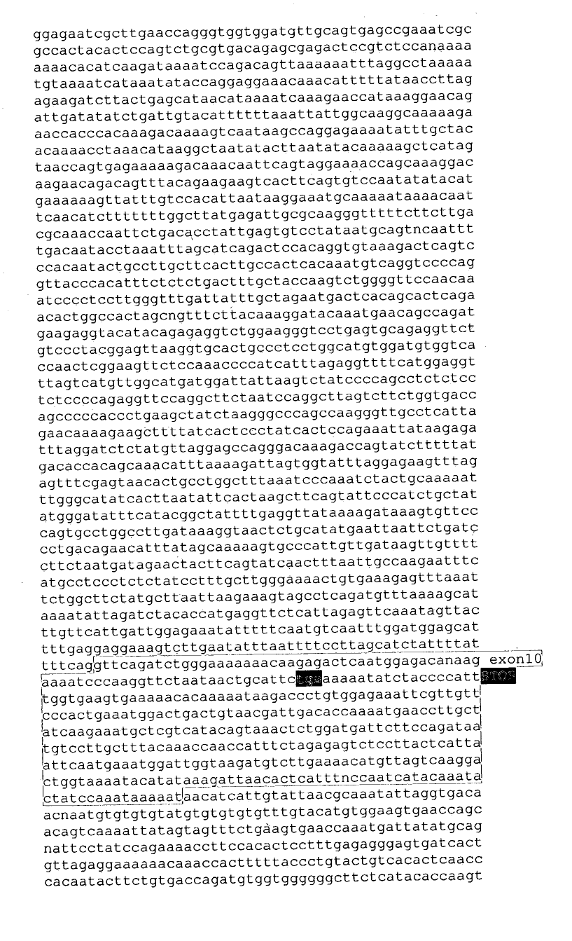

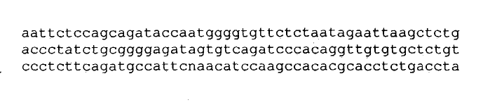

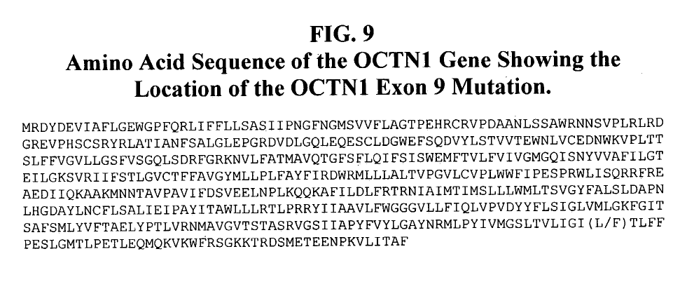

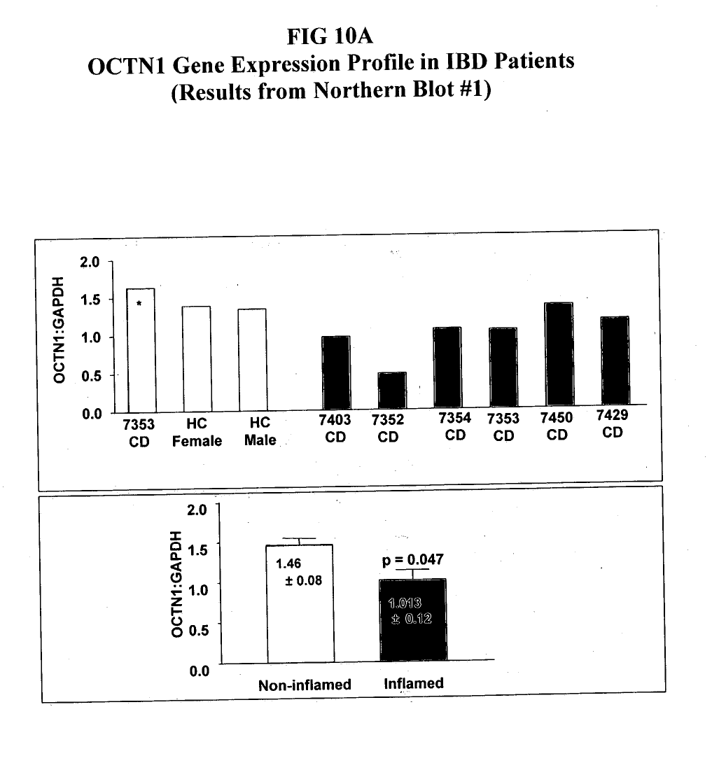

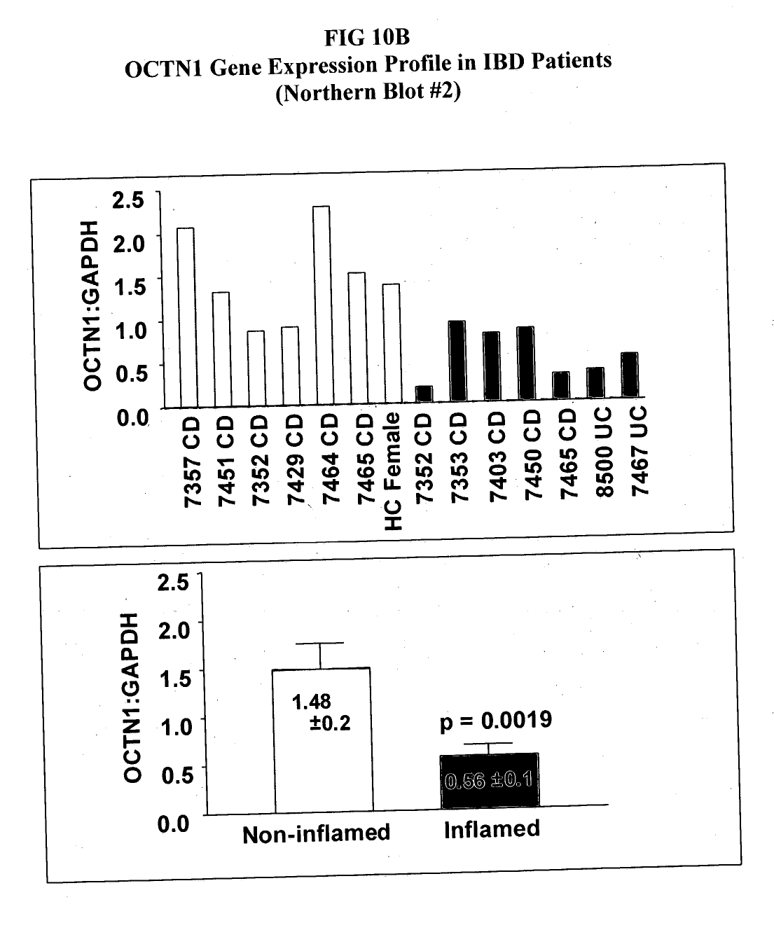

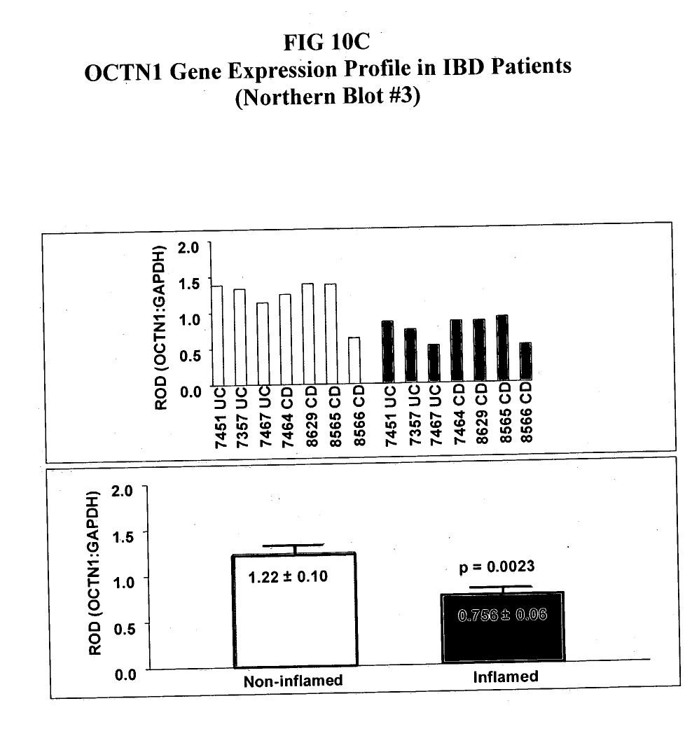

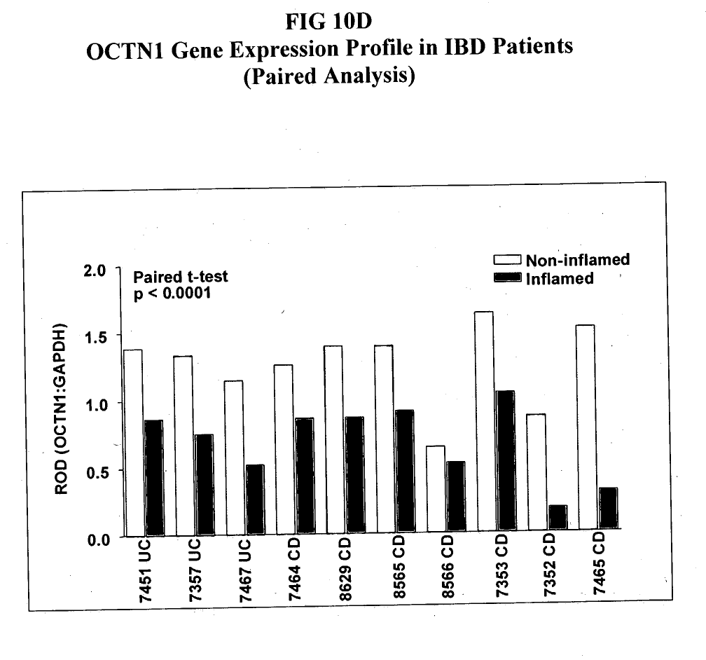

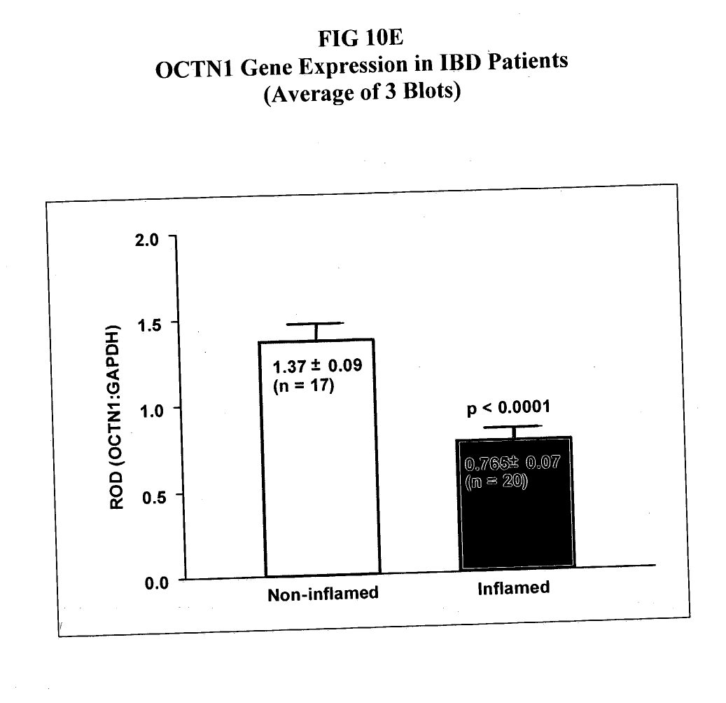

FIG. 9 shows the amino acid sequence deduced from the OCTN1 gene including position of the amino acid change caused by the OCTN1 exon 9 mutation (SEQ ID NO:38). Oligonucleotide primers for amplification of OCTN1 exon 9 mutation site include O1X9F: gtgcccagagagtcctccta (SEQ ID NO:39) and O1X9R: ttctccctaaggcattttggt (SEQ ID NO:40).

TABLE 7 shows the allele frequencies of the OCTN1 exon 9 mutation (see, FIG. 8 and FIG. 9). Alleles are listed by number (1 or 2), the DNA nucleotide present at that position (C or T), and the amino acid encoded by the codon including that nucleotide (Phe for phenylalanine or Leu for leucine). The number of chromosomes carrying each allele is shown for either unrelated healthy control individuals (“healthy” column) or Crohn's Disease patients from families with genetic risk conferred by the chromosome 5 locus previously described (“CD” column). The frequency of each allele as a percentage of the total is shown in brackets, and the total number of chromosomes (“n”) is shown at the bottom of each column. The results of a 2 by 2 contingency table chi-squared test are presented at the bottom of the table demonstrating a significant difference in allele frequency between the “healthy” and “CD” classes (chi-squared statistic of 13.1422, p value with one degree of freedom 0.0003).

TABLE 7
ALLELE FREQUENCY DATA FOR OCTN1 EXON 9 MUTATION

| allele | healthy | CD |
|---|---|---|
| 1 (T, Phe) | 74 (48%) | 42 (78%) |
| 2 (C, Leu) | 80 (52%) | 12 (22%) |
| | n = 154 | n = 54 |

chi-squared = 13.1422
p (1df) = 0.0003

The invention also provides other polynucleotide having sequences that are fragments of SEQ ID NOS:37 or 38. For example, the fragments can have sequences that that are 20 bases long (or multiples thereof).

In addition, there may be an association between the OCTN1 gene and generalized inflammatory responses. A specific protein (HSF1) may bind to the promoter region of the OCTN1 gene. Accordingly, inflammatory disease in general is within the scope of the present invention.

Thus the invention provides a method for identifying an anti-inflammatory agent. A complex is formed, either in vivo or in vitro between the heat shock factor 1 (HSF1) protein and a polynucleotide containing the OCTN1 promoter region. The complex with an agent, such as a drug, suspected of being able to dissociate the complex. Detecting the dissociation of the complex identifies the agent as being an agent that is an anti-inflammatory agent.

OCTN1 and OCTN2 Polynucleotides. The invention provides polymorphisms of OCTN1 and OCTN2. The term “polynucleotide” encompasses RNA and DNA, including cDNA, genomic DNA, and synthetic (e.g., chemically synthesized) DNA. The polynucleotide may be double-stranded or single-stranded. Where single-stranded, the polynucleotide may be the sense strand or the antisense strand. The polynucleotides of the invention thus provide material for making OCTN1 or OCTN2 polypeptides.

The polynucleotide of the invention can be used as the basis of drug development diagnostics for Inflammatory Bowel Diseases. In addition to the structural information provided by the polynucleotide of the invention is the associated information provided by the genetic analysis. Methods for using the polynucleotide of the invention in conjunction with further genetic analyses of complex genetic traits are known in the art (see, Lander E. & Kruglyak L., Nature Genet. 11: 241-247 (1995); Ott, J., Nature 379: 772-773 (1996)); as are methods for assessing the relative risk of Crohn's disease for individuals who are heterozygous, homozygous, or compound heterozygous for the identified genetic loci (see, for example, Hugot J.-P., et al., Nature 411: (2001); IBD International Genetics Consortium, Am. J. Hum. Genet. 68: 1165-1171 (2001)).

By “isolated polynucleotide” is meant DNA that is not immediately contiguous with both of the coding sequences with which it is immediately contiguous (one on the 5′ end and one on the 3′ end) in the naturally occurring genome of the organism from which it is derived. Thus, a recombinant polynucleotide could include some or all of the 5′ non-coding (e.g., promoter) sequences which are immediately contiguous to the coding sequence. The term therefore includes, for example, a recombinant DNA which is incorporated into a vector; into an autonomously replicating plasmid or virus, such as a retrovirus; or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant DNA that is part of a hybrid gene encoding additional polypeptide sequence.

By “substantially identical” is meant a polypeptide or polynucleotide having a sequence that is at least 85%, preferably 90%, and more preferably 95% or more identical to the sequence of the reference amino acid or polynucleotide sequence. For polypeptides, the length of the reference polypeptide sequence will generally be at least 16 amino acids, preferably at least 20 amino acids, more preferably at least 25 amino acids, and most preferably 35 amino acids. For polynucleotides, the length of the reference polynucleotide sequence will generally be at least 50 nucleotides, preferably at least 60 nucleotides, more preferably at least 75 nucleotides, and most preferably at least 110 nucleotides.

To determine the percent identity of two polynucleotides, the sequences are aligned for optimal comparison purposes (e.g., gaps can be introduced in the sequence of a first amino acid or polynucleotide sequence for optimal alignment with a second amino or polynucleotide sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). Preferably, the two sequences are the same length.

The determination of percent homology between two sequences can be accomplished using a mathematical algorithm. A preferred, non-limiting example of a mathematical algorithm utilized for the comparison of two sequences is the algorithm of Karlin & Altschul, Proc. Natl. Acad. Sci. USA 87:2264-2268 (1990), modified as in Karlin & Altschul, Proc. Natl. Acad. Sci. USA 90:5873-5877 (1993). Such an algorithm is incorporated into the BLASTX and BLASTN programs of Altschul et al., J. Mol. Biol. 215:403-410 (1990). BLAST nucleotide searches can be performed with the NBLAST program, score=100, wordlength=12 to obtain nucleotide sequences homologous to OCTN2 polynucleotide molecules of the invention. BLAST protein searches can be performed with the BLASTX program, score=50, wordlength=3 to obtain amino acid sequences homologous to OCTN2 protein molecules of the invention. To obtain gapped alignments for comparison purposes, Gapped BLAST can be utilized as described in Altschul et al., Nucleic Acids Res. 25: 3389-3402 (1997). Alternatively, PSI-Blast can be used to perform an iterated search that detects distant relationships between molecules. Id. When utilizing BLAST, Gapped BLAST, and PSI-Blast programs, the default parameters of the respective programs (e.g., BLASTX and BLASTN) can be used. See <http://www.ncbi.nlm.nih.gov>. Another preferred, non-limiting example of a mathematical algorithm used for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

The invention also encompasses polynucleotides that hybridize under stringent conditions to a polynucleotide encoding an OCTN1 or OCTN2 polypeptide. Examples of stringent conditions include: 1) hybridization at 50° C. in Church buffer (7% SDS, 0.5% NaHPO4, 1 mM EDTA, 1% BSA) and washing at 50° C. in 2×SSC; and 2) hybridization in 6× sodium chloride/sodium citrate (SSC) at about 45° C., followed by one or more washes in 0.2×SSC, 0.1% SDS at 50-65° C. Other stringent conditions are known to those skilled in the art and can be found in Current Protocols in Molecular Biology, (John Wiley & Sons, N.Y. 1989), 6.3.1-6.3.6. The hybridizing portions of the hybridizing polynucleotides are preferably 20, 30, 50, or 70 bases long. Preferably, the hybridizing portion of the hybridizing polynucleotide is 95% or even 98% identical to the sequence of a portion of a polynucleotide encoding an OCTN2 polypeptide. Hybridizing polynucleotides of the type described above can be used as a cloning probe, a primer (e.g., a PCR primer), or a diagnostic probe. Preferred hybridizing polynucleotides encode a polypeptide having some or all of the biological activities possessed by naturally-occurring OCTN1 or OCTN2. Hybridizing polynucleotides can be splice variants encoded by one of the OCTN1 or OCTN2 genes described herein. Thus, they may encode a protein that is shorter or longer than the various forms of OCTN1 or OCTN2 described herein. Hybridizing polynucleotides may also encode proteins that are related to OCTN1 or OCTN2 (e.g. proteins encoded by genes which include a portion having a relatively high degree of identity to an OCTN1 or OCTN2 gene described herein).

The invention also features isolated polynucleotide sequences that encode a portion of OCTN1 or OCTN2. Thus, within the invention are polynucleotides encoding fusion proteins in which a portion of OCTN1 or OCTN2 or a portion (e.g., one or more domains) thereof is fused to an unrelated protein or polypeptide (i.e., a fusion partner) to create a fusion protein. Examples of detectable markers include β-lactamase, chloramphenicol acetyltransferase (CAT), alkaline phosphatase (AP), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), β-galactosidase, and xanthine guanine phosphoribosyl-transferase (XGPRT).

The polypeptides of the invention include, but are not limited to, recombinant polypeptides, natural polypeptides produced from polynucleotides of the invention having a polymorphism disclosed herein, and synthetic polypeptides as well as polypeptides which are preproteins or proproteins. The polypeptides of the invention can be expressed fused to another polypeptide, e.g., a marker polypeptide or fusion partner. For example, the polypeptide can be fused to a hexa-histidine tag to facilitate purification of bacterially expressed protein or a hemagglutinin tag to facilitate purification of protein expressed in eukaryotic cells.

The invention features transformed cells harbouring a polynucleotide encompassed by the invention. The invention also features vectors that include a polynucleotide of the invention that is properly positioned for expression. For example, the vector can be an expression vector, and can include one or more regulatory elements. Regulatory elements that can influence the expression of the polynucleotide inserted into the vector, such as regulatory elements that direct tissue-specific expression, are well known to those of skill in the art. Examples of regulatory elements include the cytomegalovirus hCMV immediate early gene, the early promoter of SV40 adenovirus, the late promoter of SV40 adenovirus, the lac system, the trp system, the TAC system, the TRC system, the major operator and promoter regions of phage lambda, the control regions of fd coat protein, the promoter for 3-phosphoglycerate kinase, the promoters of acid phosphatase, and the promoters of the yeast α-mating factors. The vector can be a plasmid, or a virus, such as a retrovirus.

By “transformed cell” is meant a cell into which (or into an ancestor of which) has been introduced, by means of recombinant DNA techniques, a DNA molecule encoding (as used herein) an OCTN1 or OCTN2 polypeptide.

By “positioned for expression” is meant that the selected DNA molecule is positioned adjacent to one or more sequence elements which direct transcription and/or translation of the sequence such that the sequence elements can control transcription and/or translation of the selected DNA (i.e., the selected DNA is operably associated with the sequence elements). Such operably associated elements can be used to facilitate the production of an OCTN1 or OCTN2 polypeptide.

The invention also features antagonists and agonists of OCTN1 or OCTN2. Antagonists can inhibit one or more of the functions of OCTN1 or OCTN2. Suitable antagonists can include large or small molecules (e.g., organic molecules), antibodies to OCTN1 or OCTN2, and OCTN1 or OCTN2 polypeptides that compete with a native form of OCTN1 or OCTN2. Agonists of OCTN1 or OCTN2 will enhance or facilitate one or more of the functions of OCTN1 or OCTN2. Suitable agonists can include, for example, large or small molecules (e.g., organic molecules), and antibodies to OCTN1 or OCTN2. Also within the invention are polynucleotide molecules that can be used to interfere with OCTN1 or OCTN2 expression, e.g., antisense molecules and ribozymes.

The invention also features a cell that harbours a recombinant polynucleotide encoding an OCTN1 or OCTN2 polypeptide; a vector which includes a polynucleotide encoding a OCTN2 polypeptide.

The invention also features a pharmaceutical composition that includes an OCTN1 or OCTN2 polypeptide.

Methods. The invention features a method for detecting Inflammatory Bowel Diseases. This method includes: (a) obtaining a biological sample; (b) contacting the sample with probe which selectively binds an OCTN1 or OCTN2 polynucleotide; and (c) determining the amount of the probe selectively bound to said biological sample as a measure of the level of expression of v. This level, in turn, serves as a biological marker for disease activity and/or progression in individuals with Inflammatory Bowel Diseases. Thus, the invention provides a method to track or monitor expression level changes in Inflammatory Bowel Diseases as a diagnostic or prognostic tool.

In general, OCTN1 or OCTN2 proteins and fusion proteins according to the invention can be produced using the polynucleotide of the invention by transformation (transfection, transduction, or infection) of a host cell with all or part of an OCTN1-encoding or OCTN2-encoding DNA fragment (e.g., the cDNA described herein) in a suitable expression vehicle. Suitable expression vehicles include: plasmids, viral particles, and phage. For insect cells, baculovirus expression vectors are suitable. The entire expression vehicle, or a part thereof, can be integrated into the host cell genome. In some circumstances, it is desirable to employ an inducible expression vector, e.g., the LACSWITCH™ Inducible Expression System (Stratagene; La Jolla, Calif., USA).

Those skilled in the field of molecular biology will understand that any of a wide variety of expression systems can be used to provide the recombinant protein. The precise host cell used is not critical to the invention. The OCTN1 or OCTN2 protein can be produced in a prokaryotic host (e.g., E. coli or B. subtilis) or in a eukaryotic host (e.g., Saccharomyces or Pichia; mammalian cells, e.g., COS, NIH 3T3, CHO, BHK, 293, or HeLa cells; or insect cells).

Plant cells can also produce proteins and polypeptides. For plant cells viral expression vectors (e.g., cauliflower mosaic virus and tobacco mosaic virus) and plasmid expression vectors (e.g., Ti plasmid) are suitable. Such cells are available from a wide range of sources (e.g., the American Type Culture Collection (ATCC), Manassas, Va., USA.; see also, e.g., Ausubel et al, Current Protocols in Molecular Biology, (John Wiley & Sons, New York, 1994)). The methods of transformation or transfection and the choice of expression vehicle will depend on the host system selected. Transformation and transfection methods are described, e.g., in Ausubel et al. Current Protocols in Molecular Biology, (John Wiley & Sons, New York, 1994); expression vehicles may be chosen from those provided, e.g., in Cloning Vectors: A Laboratory Manual, P. H. Pouwels et al. (1985, Supp. 1987).

The host cells harboring the expression vehicle can be cultured in conventional nutrient media adapted as needed for activation of a chosen gene, repression of a chosen gene, selection of transformants, or amplification of a chosen gene.

OCTN1 or OCTN2 polypeptides can be produced as fusion proteins. For example, the expression vector pUR278 (Ruther et al., EMBO J. 2:1791, 1983), can be used to create lacZ fusion proteins. The pGEX vectors can be used to express foreign polypeptides as fusion proteins with glutathione S-transferase (GST). In general, such fusion proteins are soluble and can be easily purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect cell expression system, Autographa californica nuclear polyhidrosis virus (AcNPV), which grows in Spodoptera frugiperda cells, is used as a vector to express foreign genes. An OCTN1 or OCTN2 coding sequence can be cloned individually into non-essential regions (for example the polyhedrin gene) of the virus and placed under control of an AcNPV promoter, e.g., the polyhedrin promoter. Successful insertion of a gene encoding an OCTN1 or OCTN2 polypeptide or protein will result in inactivation of the polyhedrin gene and production of non-occluded recombinant virus (i.e., virus lacking the proteinaceous coat encoded by the polyhedrin gene). These recombinant viruses are then used to infect Spodoptera frugiperda cells in which the inserted gene is expressed (see, e.g., Smith et al., J. Virol. 46:584, 1983; Smith, U.S. Pat. No. 4,215,051).

In mammalian host cells, a number of viral-based expression systems can be utilized. In cases where an adenovirus is used as an expression vector, the OCTN1 or OCTN2 polynucleotide sequence can be ligated to an adenovirus transcription/translation control complex, e.g., the late promoter and tripartite leader sequence. This chimeric gene can then be inserted into the adenovirus genome by in vitro or in vivo recombination. Insertion into a non-essential region of the viral genome (e.g., region E1 or E3) will result in a recombinant virus that is viable and capable of expressing an OCTN1 or OCTN2 gene product in infected hosts. See, e.g., Logan, Proc. Natl. Acad. Sci. USA 81:3655 (1984).

Specific initiation signals may also be required for efficient translation of inserted polynucleotide sequences. These signals include the ATG initiation codon and adjacent sequences. In cases where an entire native OCTN1 or OCTN2 gene or cDNA, including its own initiation codon and adjacent sequences, is inserted into the appropriate expression vector, no additional translational control signals may be needed. In other cases, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators. Bittner et al., Methods in Enzymol. 153:516 (1987).

In addition, a host cell may be chosen which modulates the expression of the inserted sequences, or modifies and processes the gene product in a specific, desired fashion. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include, but are not limited to, CHO, VERO, BHK, HeLa, COS, MDCK, 293, 3T3, WI38, and in particular, choroid plexus cell lines.

Any technique known in the art can be used to introduce a OCTN2 transgene into animals to produce the founder lines of transgenic animals. Such techniques include, but are not limited to, pronuclear microinjection (U.S. Pat. No. 4,873,191); retrovirus mediated gene transfer into germ lines (Van der Putten et al., Proc. Natl. Acad. Sci., USA 82:6148 (1985)); gene targeting into embryonic stem cells (Thompson et al., Cell 56:313 (1989)); and electroporation of embryos (Lo, Mol. Cell. Biol. 3:1803, 1983).

The present invention provides for transgenic animals that carry the OCTN2 transgene in all their cells, as well as animals that carry the transgene in some, but not all of their cells, i.e., mosaic animals. The transgene can be integrated as a single transgene or in concatamers, e.g., head-to-head tandems or head-to-tail tandems. The transgene can also be selectively introduced into and activated in a particular cell type. Lasko et al., Proc. Natl. Acad. Sci. USA 89:6232 (1992). The regulatory sequences required for such a cell-type specific activation will depend upon the particular cell type of interest, and will be apparent to those of skill in the art.

When it is desired that the OCTN1 or OCTN2 transgene be integrated into the chromosomal site of the endogenous OCTN2 gene, gene targeting is preferred. Briefly, when such a technique is to be used, vectors containing some nucleotide sequences homologous to an endogenous OCTN1 or OCTN2 gene are designed for the purpose of integrating, via homologous recombination with chromosomal sequences, into and disrupting the function of the nucleotide sequence of the endogenous gene. The transgene also can be selectively introduced into a particular cell type, thus inactivating the endogenous OCTN1 or OCTN2 gene in only that cell type. Gu et al., Science 265:103 (1984). The regulatory sequences required for such a cell-type specific inactivation will depend upon the particular cell type of interest, and will be apparent to those of skill in the art.

Once transgenic animals have been generated, the expression of the recombinant OCTN1 or OCTN2 gene can be assayed utilizing standard techniques. Initial screening may be accomplished by Southern blot analysis or PCR techniques to analyse animal tissues to assay whether integration of the transgene has taken place. The level of mRNA expression of the transgene in the tissues of the transgenic animals may also be assessed using techniques which include, but are not limited to, Northern blot analysis of tissue samples obtained from the animal, in situ hybridisation analysis, and RT-PCR. Samples of OCTN1 or OCTN2 gene-expressing tissue also can be evaluated immunocytochemically using antibodies specific for the OCTN1 or OCTN2 transgene product.

Antisense Polynucleotides. Antisense approaches involve the design of oligonucleotides (either DNA or RNA) that are complementary to OCTN1 or OCTN2 mRNA. The antisense oligonucleotides bind to the complementary OCTN1 or OCTN2 mRNA transcripts and prevent translation. Absolute complementarity, although preferred, is not required. A sequence “complementary” to a portion of an RNA, as referred to herein, means a sequence having sufficient complementarity to be able to hybridise with the RNA, forming a stable duplex; in the case of double-stranded antisense polynucleotides, a single strand of the duplex DNA may be tested, or triplex formation may be assayed. The ability to hybridise will depend on both the degree of complementarily and the length of the antisense polynucleotide. Generally, the longer the hybridising polynucleotide, the more base mismatches with an RNA it may contain and still form a stable duplex (or triplex, as the case may be). One skilled in the art can ascertain a tolerable degree of mismatch by use of standard procedures to determine the melting point of the hybridised complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs recently have been shown to be effective at inhibiting translation of mRNAs as well. Wagner, Nature 372:333 (1984). Thus, oligonucleotides complementary to the 5′- or 3′-non-translated, non-coding regions of the OCTN1 or OCTN2 gene, e.g., the human genes shown in FIG. 8 and FIG. 6, could be used in an antisense approach to inhibit translation of endogenous OCTN2 mRNA. Oligonucleotides complementary to the 5′ untranslated region of the mRNA should include the complement of the AUG start codon.

Antisense oligonucleotides complementary to mRNA coding regions are less efficient inhibitors of translation but could be used in accordance with the invention. Whether designed to hybridise to the 5′-, 3′-, or coding region of OCTN1 or OCTN2 mRNA, antisense polynucleotides should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridisation to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridisation, etc. The oligonucleotide may include other appended groups such as peptides (e.g., for targeting host cell receptors in vivo), or agents facilitating transport across the cell membrane (as described, e.g., in Letsinger et al., Proc. Natl. Acad. Sci. USA 86:6553 (1989); Lemaitre et al., Proc. Natl. Acad. Sci. USA 84:648 (1987); PCT Publication No. WO 88/09810) or the blood-brain barrier (see, e.g., PCT Publication No. WO 89/10134), or hybridisation-triggered cleavage agents (see, e.g., Krol et al., BioTechniques 6:958 (1988)), or intercalating agents (see, e.g., Zon, Pharm. Res. 5:539 (1988)). To this end, the oligonucleotide can be conjugated to another molecule, e.g., a peptide, hybridisation triggered cross-linking agent, transport agent, or hybridisation-triggered cleavage agent.

The antisense oligonucleotide may comprise at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethyl-aminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-uracil-5-oxyacetic acid (v), wybutoxosine, pseudouracil, queosine, 2-thiocytosine, 5-methyl-2-theouracil, 2-thiouracil, 4-thiouracil, 5-methyluracil, uracil-5-oxyacetic acid methylester, uracil-5-oxyacetic acid (v), 5-methyl-2-thiouracil, 2-(3-amino-3-N-2-carboxypropl) uracil, (acp3)w, and 2,6-diaminopurine.

The antisense oligonucleotide may also have at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal, or an analog of any of these backbones.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other. Gautier et al., Nucl. Acids. Res. 15:6625 (1987). The oligonucleotide is a 2′-0-methylribonucleotide (Inoue et al., Nucl. Acids Res. 15:6131 (1987)), or a chimeric RNA-DNA analog (Inoue et al., FEBS Lett. 215:327 (1987)).

Antisense oligonucleotides of the invention can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. Nucl. Acids Res. 16:3209 (1988), and methylphosphonate oligonucleotides can be prepared by use of controlled pore glass polymer supports. Sarin et al., Proc. Natl. Acad. Sci. USA 85:7448 (1988).

While antisense nucleotides complementary to the OCTN1 or OCTN2 coding region sequence could be used, those complementary to the transcribed untranslated region are most preferred.

The antisense molecules can be delivered to cells that express OCTN1 or OCTN2 in vivo, e.g., cells of the gastrointestinal tract and the immune system. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically to achieve intracellular concentrations of the antisense molecule sufficient to suppress translation of endogenous mRNAs and are known in the art.

Ribozymes. Ribozyme molecules designed to catalytically cleave OCTN1 or OCTN2 mRNA transcripts also can be used to prevent translation of OCTN2 mRNA and expression of OCTN2 (see, e.g., PCT Publication No. WO 90/11364; Saraver et al., Science 247:1222 (1990)). While various ribozymes that cleave mRNA at site-specific recognition sequences can be used to destroy OCTN1 or OCTN2 mRNAs, the use of hammerhead ribozymes is preferred. Hammerhead ribozymes cleave mRNAs at locations dictated by flanking regions that form complementary base pairs with the target mRNA. The sole requirement is that the target its mRNA have the following sequence of two bases: 5′-UG-3′. The construction and production of hammerhead ribozymes is well known in the art. Haseloff et al., Nature 334:585 (1988).

The ribozymes of the invention also include RNA endoribonucleases (hereinafter “Cech-type ribozymes”), such as the one that occurs naturally in Tetrahymena thermophila (known as the IVS or L-19 IVS RNA), and which has been extensively described by Cech and his collaborators. Zaug et al., Science 224:574 (1984); Zaug et al., Science 231:470 (1986); Zug et al., Nature 324:429 (1986); PCT Application No. WO 88/04300; and Been et al., Cell 47:207 (1986). The Cech-type ribozymes have an eight base-pair sequence that hybridises to a target RNA sequence, whereafter cleavage of the target RNA takes place. The invention encompasses those Cech-type ribozymes that target eight base-pair active site sequences present in OCTN2.

Methods for Modulating OCTN1 or OCVN2 Expression. Endogenous OCTN1 or OCTN2 gene expression can also be reduced by inactivating or “knocking out” the OCTN1 or OCTN2 gene or its promoter using targeted homologous recombination (see, e.g., U.S. Pat. No. 5,464,764). For example, a mutant, non-functional OCTN2 (or a completely unrelated DNA sequence) flanked by DNA homologous to the endogenous OCTN2 gene (either the coding regions or regulatory regions of the OCTN2 gene) can be used, with or without a selectable marker and/or a negative selectable marker, to transfect cells that express OCTN2 in vivo. Insertion of the DNA construct, via targeted homologous recombination, results in inactivation of the OCTN1 or OCTN2 gene. Such approaches are particularly suited for use in the agricultural field where modifications to ES (embryonic stem) cells can be used to generate animal offspring with an inactive OCTN1 or OCTN2. However, this approach can be adapted for use in humans, provided the recombinant DNA constructs are directly administered or targeted to the required site in vivo using appropriate viral vectors, e.g., herpes virus vectors for delivery to brain tissue; e.g., the arcuate nucleus or the choroid plexus.

In another embodiment, modulators of OCTN1 or OCTN2 expression are identified in a method in which a cell is contacted with a candidate compound and the expression of OCTN1 or OCTN2 mRNA or protein in the cell is determined. The level of expression of OCTN1 or OCTN2 mRNA or protein in the presence of the candidate compound is compared to the level of expression of OCTN1 or OCTN2 mRNA or protein in the absence of the candidate compound. The candidate compound can then be identified as a modulator of OCTN1 or OCTN2 expression based on this comparison. For example, when expression of OCTN1 or OCTN2 mRNA or protein is greater (statistically significantly greater) in the presence of the candidate compound than in its absence, the candidate compound is identified as a stimulator of OCTN1 or OCTN2 mRNA or protein expression. Alternatively, when expression of OCTN1 or OCTN2 mRNA or protein is less (statistically significantly less) in the presence of the candidate compound than in its absence, the candidate compound is identified as an inhibitor of OCTN1 or OCTN2 mRNA or protein expression. The level of OCTN1 or OCTN2 mRNA or protein expression in the cells can be determined by methods described herein for detecting OCTN1 or OCTN2 mRNA or protein.

Examples of methods for the synthesis of molecular libraries can be found in the art, for example in: DeWitt et al., Proc. Natl. Acad. Sci. USA 90:6909 (1993); Erb et al., Proc. Natl. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33:2059 (1994); Carell et al., Angew. Chem. Int. Ed. Engl. 33:2061 (1994); and Gallop et al., J. Med. Chem. 37:1233 (1994).

Libraries of compounds may be presented in solution (e.g., Houghten, Bio/Techniques 13:412-421 (1992)), or on beads (Lam, Nature 354:82-84 (1991)), chips (Fodor, Nature 364:555-556 (1993)), bacteria (U.S. Pat. No.5,223,409), spores (U.S. Pat. Nos. 5,571,698; 5,403,484; and 5,223,409), plasmids (Cull et al., Proc. Natl. Acad. Sci. USA 89:1865-1869 (1992)) or phage (Scott & Smith, Science 249:386-390 (1990); Devlin, Science 249:404-406 (1990); Cwirla et al., Proc. Natl. Acad. Sci. USA 87:6378-6382 (1990); and Felici, J. Mol. Biol. 222:301-310 (1991).

Diagnostic Applications. The polynucleotides, polypeptides, and antibodies of the invention are useful for identifying those compartments of mammalian cells that contain proteins important to the function of OCTN1 or OCTN2. Antibodies specific for OCTN1 or OCTN2 may be produced as described above. The normal location of the protein is then determined either in situ or using fractionated cells by any standard immunological or immunohistochemical procedure (see, e.g., Ausubel et al., supra; Bancroft & Stevens, Theory and Practice of Histological Techniques, (Churchill Livingstone, 1982).

Alternatively, OCTN1 or OCTN2 expression can be assayed by standard Northern blot analysis or can be aided by PCR (see, e.g., Ausubel et al., supra; PCR Technology: Principles and Applications for DNA Amplification, ed., H. A. Ehrlich (Stockton Press, N.Y.). Also, see above for working examples. If desired or necessary, analysis can be carried out to detect point mutations in the OCTN2 sequence (for example, using well-known polynucleotide mismatch detection techniques). All of the above techniques are enabled by the OCTN1 or OCTN2 sequences described herein.

Accordingly, the polynucleotides, polypeptides and antibodies of the invention can be used in a method for determining whether a patient has a disorder associated with abnormal expression of OCTN1 or OCTN2. The method can be carried out by quantitating the level of expression of OCTN1 or OCTN2 in a biological sample obtained from the patient. As a control, the quantitation can be carried out using a biological sample obtained from a subject who is healthy.

OCTN1 or OCTN2 expression can be assessed at the level of gene expression, for example, by quantitating the level of OCTN1 or OCTN2 mRNA expression in the biological sample, or at the level of protein expression, by quantitating the level of OCTN1 or OCTN2 protein expressed. Quantitation can be carried out using the techniques described above, which are well within the abilities of those of skill in the art to perform.

Should it be determined that a patient has a disorder that is associated with abnormal expression or activity of OCTN1 or OCTN2, the patient can be given a compound that modulates that expression or activity. For example, the patient can receive a compound such as a small molecule, an antisense polynucleotide molecule, or a ribozyme, that inhibits the expression of OCTN2. The patient can also receive a compound that inhibits the activity of OCTN1 or OCTN2. An antibody that specifically binds OCTN1 or OCTN2 can be used for this purpose. Alternatively, the patient can receive a compound that enhances the expression or activity of OCTN2. Compounds that inhibit or enhance the expression or activity of OCTN2 can include synthetic molecules. These methods of treatment can be used to treat Inflammatory Bowel Diseases and related disorders associated with cellular proliferation.

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but by the claims appended hereto.

What is claimed is:

1. An isolated OCTN1 polynucleotide, wherein the OCTN1 polynucleotide has a nucleotide sequence comprising a sequence selected from the group consisting of SEQ ID NO:7; fragments of SEQ ID NO:7, where in the fragments are at least twenty nucleotides in length and contain a sequence corresponding to the L503F residue of SEQ ID NO:7; SEQ ID NO:9; SEQ ID NO:10; SEQ ID NO:21; SEQ ID NO:22; SEQ ID NO:39; and SEQ ID NO:40.

2. The OCTN1 polynucleotide of claim 1, further comprising a polynucleotide vector in which the OCTN1 polynucleotide is situated.

3. An article of manufacture, comprising:
(a) a polynucleotide hybridisation probe that is homologous to a OCTN 1 polynucleotide; and specifically binds under stringent hybridization conditions to the OCTN1 polynucleotide, wherein the OCTN1 polynucleotide has a nucleotide sequence comprising a sequence selected from the group consisting of SEQ ID NO:1; fragments of SEQ ID NO:1, where in the fragments are at least twenty nucleotides in length; SEQ ID NO:36; fragments of SEQ ID NO:36, where in the fragments are at least twenty nucleotides in length; SEQ ID NO:37; fragments of SEQ ID NO:37, where in the fragments are at least twenty nucleotides in length; SEQ ID NO:7; fragments of SEQ ID NO:7, where in the fragments are at least twenty nucleotides in length and contain a sequence corresponding to the L503F residue of SEQ ID NO:7; SEQ ID NO:9; SEQ ID NO:10; SEQ ID NO:21; SEQ ID NO:22; SEQ ID NO:39; and SEQ ID NO:40; and
(b) a set of instruction regarding the use of the polynucleotide hybridization probe in the diagnosis of Inflammatory Bowel Disease (IBD).

4. An isolated OCTN2 polynucleotide, wherein the OCTN2 polynucleotide has a nucleotide sequence comprising a sequence selected from the group consisting of SEQ ID NO:8; fragments of SEQ ID NO:8, where in the fragments are at least twenty nucleotides in length and contain a sequence corresponding to the G-207C residue of SEQ ID NO:8; SEQ ID NO:5; SEQ ID NO:6; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:29; SEQ ID NO:30; SEQ ID NO:31; SEQ ID NO:32; SEQ ID NO:33; SEQ ID NO:34; and SEQ ID NO:35.

5. The OCTN2 polynucleotide of claim 5, further comprising a polynucleotide vector in which the OCTN2 polynucleotide is situated.

6. An article of manufacture, comprising:
(a) a polynucleotide hybridization probe that is homologous to an OCTN2 polynucleotide and specifically binds under stringent hybridization conditions to the OCTN2 polynucleotide, OCTN2 polynucleotide has a nucleotide sequence comprising a sequence selected from the group consisting of SEQ ID NO:3; fragments of SEQ ID NO:3, where in the fragments are at least twenty nucleotides in length; SEQ ID NO:23; fragments of SEQ ID NO:23, where in the fragments are at least twenty nucleotides in length; SEQ ID NO:24; SEQ ID NO:25; SEQ ID NO:26; SEQ ID NO:27; SEQ ID NO:28; SEQ ID NO:8; fragments of SEQ ID NO:8, where in the fragments are at least twenty nucleotides in length and contain a sequence corresponding to the G-207C residue of SEQ ID NO:8; SEQ ID NO:5; SEQ ID NO:6; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:29; SEQ ID NO:30; SEQ ID NO:31; SEQ ID NO:32; SEQ ID NO:33; SEQ ID NO:34; and SEQ ID NO:35; and
(b) a set of instruction regarding the use of the polynucleotide hybridization probe in the diagnosis of Inflammatory Bowel Disease (IBD).

7. A method for determining a susceptibility to Inflammatory Bowel Disease (IBD) in a mammal, comprising:
(a) obtaining a biological sample from a mammal;
(b) contacting the biological sample with polynucleotide probe that selectively binds an OCTN1 polynucleotide or an OCTN2 polynucleotide; and
(c) determining the expression of the OCTN1 polynucleotide or the OCTN2 polynucleotide as a measure of susceptibility of the mammal to Inflammatory Bowel Disease.

8. The method of claim 7, wherein the expression of the OCTN1 polynucleotide or the OCTN2 polynucleotide in the biological sample is reduced in mammals having a susceptibility to Inflammatory Bowel Disease as compared with mammals that are not susceptibel to Inflammatory Bowel Disease.

9. The method of claim 7, wherein the biological sample is selected from the group consisting of gastrointestinal tract tissue and immune system tissue.

10. The method of claim 7, wherein the biological sample is selected from inflamed tissue.

11. The method of claim 7, wherein the mammal is human.

12. The method of claim 7, wherein the Inflammatory Bowel Disease is selected from the group consisting of Crohn's disease (CD), indeterminate colitis (IC) and ulcerative colitis (UC).

13. The method of claim 7, wherein the determination of the expression is by Northern analysis.

14. The method of claim 7, wherein the determination of the expression is by polymerase chain reaction.

15. The method of claim 7, wherein the expression of both the OCTN1 polynucleotide and the OCTN2 polynucleotide in the biological sample is determined.

16. The method of claim 7, wherein the basal transcription of the OCTN2 gene is downregulated.

17. The method of claim 7, wherein the transcription of the OCTN2 gene induced either by heat shock or by arachidonic acid is downregulated.

18. The method of claim 7, wherein both the basal transcription of the OCTN2 gene and the transcription of the OCTN2 gene induced either by heat shock or by arachidonic acid is downregulated.

19. A method for determining a susceptibility to inflammatory bowel disease (IBD), in a human, comprising:
(a) determining the polynucleotide sequence of the OCTN1 gene or the OCTN2 gene for a human; and
(a) determining that the polynucleotide sequence of the OCTN1 gene or the OCTN2 gene for the human contains a polymorphism that results in reduced expression of OCTN1 or OCTN2 in the human.

20. The method of claim 19, wherein the polynucleotide sequences of both the OCTN1 gene and the OCTN2 gene are determined for the human.

21. The method of claim 19, wherein the polynucleotide sequence of the OCTN1 gene contains at least one L503F polymorphism.

22. The method of claim 19, wherein the polynucleotide sequence of the OCTN1 gene significantly reduces the ability of the encoded OCTN1 polypeptide to transport carnitine.

23. The method of claim 19, wherein the polynucleotide sequence of the OCTN2 gene contains at least one polymorphism selected from the group consisting of G-207C, 410 and TA.

24. The method of claim 19, wherein the polynucleotide sequence of the OCTN2 gene contains at least one polymorphism in the OCTN2 promoter region.

25. The method of claim 19, wherein the polymorphism alters the binding of heat shock transcription factor 1 (HSF1) to the OCTN2 promoter.

26. The method of claim 19, wherein the polymorphism of the OCTN2 gene downregulates basal transcription.

27. The method of claim 19, wherein the polymorphism of the OCTN2 gene downregulates transcription induced either by heat shock or by arachidonic acid.

28. The method of claim 19, wherein the polymorphism in the OCTN2 gene downregulates both basal transcription and transcription induced either by heat shock or by arachidonic acid.

29. A method for identifying an anti-inflammatory agent, comprising:
(a) forming a complex between the heat shock factor 1 (HSF1) protein and a polynucleotide comprising an OCTN2 promoter region;
(b) contacting the complex with an agent suspected of dissociating the complex; and
(c) detecting the dissociation of the complex, wherein the dissociation of the complex identifies the agent as being an agent that is an anti-inflammatory agent.

30. A method for detecting a nucleotide polymorphism in a mammalian OCTN1 gene or a mammalian OCTN2 gene associated with an Inflammatory Bowel Disease, which method comprises:
detecting a variation in a sequence of a gene encoding OCTN1 or OCTN2 obtained from a mammal diagnosed with or suspected of having Inflammatory Bowel Disease.

31. The method of claim 30, wherein the mammal is human.

32. The method of claim 30, wherein the expression of the OCTN1 gene is significantly downregulated in inflamed tissue in the mammal.

33. The method of claim 30, wherein the expression of the OCTN2 gene is significantly downregulated in inflamed tissue in the mammal.

34. A method for identifying a compound for treating an Inflammatory Bowel Disease, which method comprises:
(a) contacting a test compound with a system for measuring carnitine transport activity, which system comprises an OCTN1 or OCTN2 polypeptide or a functional fragment of an OCTN1 or OCTN2 polypeptide, and a substrate for measuring carnitine transport by the system; and
(b) detecting an increase in the carnitine transport activity of the in the presence of the test compound compared to the carnitine transport activity in the absence of the test compound.

35. The method of claim 34, wherein the test compound is a small molecule.

36. The method of claim 34, wherein the test compound is an intracellular factor that binds to the OCTN1 or the OCTN2 polypeptide.

37. The method of claim 34, wherein the test compound is an extracellular factor that binds to the OCTN1 or the OCTN2 polypeptide.

38. The method of claim 34, wherein the substrate for measuring carnitine transport is selected from the group consisting of consisting of carnitine, tetraethyl ammonium (TEA) and other compounds transported by the OCTN1 or the OCTN2 transporters.

39. The method of claim 34, wherein the OCTN1 polypeptide is encoded by a nucleotide sequence comprising a sequence selected from the group consisting of SEQ ID NO:7 and functional fragments thereof containing a sequence corresponding to the L503F residue of SEQ ID NO:7.

40. The method of claim 34, wherein the OCTN2 polypeptide is encoded by a nucleotide sequence comprising a sequence selected from the group consisting of SEQ ID NO:8;
and functional fragments thereof containing a sequence corresponding to the G-207C residue of SEQ ID NO:8.

41. A method for identifying a compound for modulating carnitine transport, which method comprises:
(a) contacting a test compound with a system for measuring carnitine transport activity, which system comprises an OCTN1 or an OCTN2 polypeptide or a functional fragment of an OCTN1 or an OCTN2 polypeptide, and a substrate for measuring carnitine transport by the system; and
(b) detecting a modulation in the carnitine transport activity of the in the presence of the test compound compared to the carnitine transport activity in the absence of the test compound.

42. The method of claim 41, wherein the compound enhances the transporter function of OCTN1.

43. The method of claim 41, wherein the compound reduces or blocks the transporter function of OCTN1.

44. The method of claim 41, wherein the compound enhances the transporter function of OCTN2.

45. The method of claim 41, wherein the compound reduces or blocks the transporter function of OCTN2.

46. A method for identifying a compound for treating an Inflammatory Bowel Disease, which method comprises:
(a) obtaining a transgenic mammal or a muatnt mammal, which mamal has a nucleotide polymorphism in an OCTN1 gene or the OCTN2 gene, wherein the polymorphism in the OCTN1 gene or the OCTN2 gene reduces the tissue expression of the OCTN1 gene or the OCTN2 gene;
(b) contacting the mammal with a test compound; and
(b) detecting an improvement in a condition of the mammal in response to the test compound, wherein the condition is a symptom of an Inflammatory Bowel Disease.

47. The method of claim 46, wherein the mamal has a mutation in both the OCTN1 gene and the OCTN2 gene.

48. The method of claim 46, wherein the Inflammatory Bowel Disease is selected from the group consisting of Crohn's disease (CD), indeterminate colitis (IC) and ulcerative colitis (UC).


SEQUENCE LISTING (42 sequences)

SEQ ID NO: 1 - DNA, 2214 bp, Homo sapiens
  CDS (166)..(1821)
cctgtttcccaggaacggtccccggcttcgcgccccaatttctaacagcctgcctgtcccccgggaacgttctaacatccttggggagcgccccagctacaagacactgtcctgagaacgctgtcatcacccgtagttgcaagtttcggagcggcagtgggaagcatgcgggactacgacgaggtgatcgccttcctgggcgagtgggggcccttccagcgcctcatcttcttcctgctcagcgccagcatcatccccaatggcttcaatggtatgtcagtcgtgttcctggcggggaccccggagcaccgctgtcgagtgccggacgccgcgaacctgagcagcgcctggcgcaacaacagtgtcccgctgcggctgcgggacggccgcgaggtgccccacagctgcagccgctaccggctcgccaccatcgccaacttctcggcgctcgggctggagccggggcgcgacgtggacctggggcagctggagcaggagagctgcctggatggctgggagttcagccaggacgtctacctgtccaccgtcgtgaccgagtggaatctggtgtgtgaggacaactggaaggtgcccctcaccacctccctgttcttcgtaggcgtgctcctcggctccttcgtgtccgggcagctgtcagacaggtttggcaggaagaacgttctcttcgcaaccatggctgtacagactggcttcagcttcctgcagattttctccatcagctgggagatgttcactgtgttatttgtcatcgtgggcatgggccagatctccaactatgtggtagccttcatactaggaacagaaattcttggcaagtcagttcgtattatattctctacattaggagtgtgcacattttttgcagttggctatatgctgctgccactgtttgcttacttcatcagagactggcggatgctgctgctggcgctgacggtgccgggagtgctgtgtgtcccgctgtggtggttcattcctgaatctccccgatggctgatatcccagagaagatttagagaggctgaagatatcatccaaaaagctgcaaaaatgaacaacatagctgtaccagcagtgatatttgattctgtggaggagctaaatcccctgaagcagcagaaagctttcattctggacctgttcaggactcggaatattgccataatgaccattatgtctttgctgctatggatgctgacctcagtgggttactttgctctgtctctggatgctcctaatttacatggagatgcctacctgaactgtttcctctctgccttgattgaaattccagcttacattacagcctggctgctattgcgaaccctgcccaggcgttatatcatagctgcagtactgttctggggaggaggtgtgcttctcttcattcaactggtacctgtggattattacttcttatccattggtctggtcatgctgggaaaatttgggatcacctctgctttctccatgctgtatgtcttcactgctgagctctacccaaccctggtcaggaacatggcggtgggggtcacatccacggcctccagagtgggcagcatcattgccccctactttgtttacctcggtgcttacaacagaatgctgccctacatcgtcatgggtagtctgactgtcctgattggaatcctcaccctttttttccctgaaagtttgggaatgactcttccagaaaccttagagcagatgcagaaagtgaaatggttcagatctgggaaaaaaacaagagactcaatggagacagaagaaaatcccaaggttctaataactgcattctgaaaaaatatctaccccatttggtgaagtgaaaaacagaaaaataagaccctgtggagaaattcgttgttcccactgaaatggactgactgtaacgattgacaccaaaatgaaccttgctatcaagaaatgctcgtcatacagtaaactctggatgattcttccagataatgtccttgctttacaaaccaaccatttctagagagtctccttactcattaattcaatgaaatggattggtaagatgtcttgaaaacatgttagtcaaggactggtaaaatacatataaagattaacactcatttccaatcatacaaatactatccaaataaaaataacatcattgtattaacgcaaatattaggtgacaacaaaaaaaaaaaaaaaaaaaaaaaa

SEQ ID NO: 2 - protein, 551 aa, Homo sapiens
MRDYDEVIAFLGEWGPFQRLIFFLLSASIIPNGFNGMSVVFLAGTPEHRCRVPDAANLSSAWRNNSVPLRLRDGREVPHSCSRYRLATIANFSALGLEPGRDVDLGQLEQESCLDGWEFSQDVYLSTVVTEWNLVCEDNWKVPLTTSLFFVGVLLGSFVSGQLSDRFGRKNVLFATMAVQTGFSFLQIFSISWEMFTVLFVIVGMGQISNYVVAFILGTEILGKSVRIIFSTLGVCTFFAVGYMLLPLFAYFIRDWRMLLLALTVPGVLCVPLWWFIPESPRWLISQRRFREAEDIIQKAAKMNNIAVPAVIFDSVEELNPLKQQKAFILDLFRTRNIAIMTIMSLLLWMLTSVGYFALSLDAPNLHGDAYLNCFLSALIEIPAYITAWLLLRTLPRRYIIAAVLFWGGGVLLFIQLVPVDYYFLSIGLVMLGKFGITSAFSMLYVFTAELYPTLVRNMAVGVTSTASRVGSIIAPYFVYLGAYNRMLPYIVMGSLTVLIGILTLFFPESLGMTLPETLEQMQKVKWFRSGKKTRDSMETEENPKVLITAF

SEQ ID NO: 3 - DNA, 3252 bp, Homo sapiens
  CDS (222)..(1895)
gcggcccaggcccgcaaccttccctggtcgtgcgccctatgtaaggccagccgcggcaggaccaaggcggcggtgtcagctcgcgagcctaccctccgcggacggtcttgggtcgcctgctgcctggcttgcctggtcggcggcgggtgccccgcgcgcacgcgcaaagcccgccgcgttccccgaccccaggccgcgctctgtgggcctctgagggcggcatgcgggactacgacgaggtgaccgccttcctgggcgagtgggggcccttccagcgcctcatcttcttcctgctcagcgccagcatcatccccaatggcttcaccggcctgtcctccgtgttcctgatagcgaccccggagcaccgctgccgggtgccggacgccgcgaacctgagcagcgcctggcgcaaccacactgtcccactgcggctgcgggacggccgcgaggtgccccacagctgccgccgctaccggctcgccaccatcgccaacttctcggcgcttgggctggagccggggcgcgacgtggacctggggcagctggagcaggagagctgtctggatggctgggagttcagtcaggacgtctacctgtccaccattgtgaccgagtggaacctggtgtgtgaggacgactggaaggccccactcacaatctccttgttcttcgtgggtgtgctgttgggctccttcatttcagggcagctgtcagacaggtttggccggaagaatgtgctgttcgtgaccatgggcatgcagacaggcttcagcttcctgcagatcttctcgaagaattttgagatgtttgtcgtgctgtttgtccttgtaggcatgggccagatctccaactatgtggcagcatttgtcctggggacagaaattcttggcaagtcagttcgtataatattctctacgttaggagtgtgcatattttatgcatttggctacatggtgctgccactgtttgcttacttcatccgagactggcggatgctgctggtggcgctgacgatgccgggggtgctatgcgtggcactctggtggttcatccctgagtccccccgatggctcatctctcagggacgatttgaagaggcagaggtgatcatccgcaaggctgccaaagccaatgggattgttgtgccttccactatctttgacccgagtgagttacaagacctaagttccaagaagcagcagtcccacaacattctggatctgcttcgaacctggaatatccggatggtcaccatcatgtccataatgctgtggatgaccatatcagtgggctattttgggctttcgcttgatactcctaacttgcatggggacatctttgtgaactgcttcctttcagcgatggttgaagtcccagcatatgtgttggcctggctgctgctgcaatatttgccccggcgctattccatggccactgccctcttcctgggtggcagtgtccttctcttcatgcagctggtacccccagacttgtattatttggctacagtcctggtgatggtgggcaagtttggagtcacggctgccttttccatggtctacgtgtacacagccgagctgtatcccacagtggtgagaaacatgggtgtgggagtcagctccacagcatcccgcctgggcagcatcctgtctccctacttcgtttaccttggtgcctacgaccgcttcctgccctacattctcatgggaagtctgaccatcctgacagccatcctcaccttgtttctcccagagagcttcggtaccccactcccagacaccattgaccagatgctaagagtcaaaggaatgaaacacagaaaaactccaagtcacacaaggatgttaaaagatggtcaagaaaggcccacaatccttaaaagcacagccttctaacatcgcttccagtaagggagaaactgaagaggaaagactgtcttgccagaaatggccagcttgtgcagactccgagtccttcagtgacaaaggcctttgctgtttgtcctcttgacctgtgtctgacttgctcctggatgggcacccacactcagaggctacatatggccctagagcaccaccttcctctagggacactggggctacctacagacaacttcatctaagtcctaactattacaatgatggactcagcacctccaaagcagttaatttttcactagaaccagtgagatctggaggaatgtgagaagcatatgctaaatgtacattttaattttagactacttgaaaaggcccctaataaggctagaggtctaagtcccccacccctttccccactcccctctagtggtgaactttagaggaaaaggaagtaattgcacaaggagtttgattcttaccttttctcagttacagaggacattaactggatcattgcttccccagggcaggagagcgcagagctagggaaagtgaaaggtaatgaagatggagcagaatgagcagatgcagatcaccagcaaagtgcactgatgtgtgagctcttaagaccactcagcatgacgactgagtagacttgtttacatctgatcaaagcactgggcttgtccaggctcataataaatgctccattgaatctactattcttgttttccactgctgtggaaacctccttgctactatagcgtcttatgtatggtttaaaggaaatttatcaggtgagagagatgagcaacgttgtcttttctctcaaagctgtaatgtgggttttgttttattgtttatttgtttgttgttgtatccttttctccttgttatttgcccttcagaatgcacttgggaaaggctggttccttagcctcctggtttgtgtctttttttttttttttttaaacacagaatcactctggcaattgtctgcagctgccactggtgcaaggccttaccagccctagcctctagcacttctctaagtgccaaaaacagtgtcattgtgtgtgttcctttcttgatacttagtcatgggaggatattacaaaaaagaaatttaaattgtgttcatagtctttcagagtagctcactttagtcctgtaactttattgggtgatattttgtgttcagtgtaattgtcttctctttgctgattatgttaccatggtactcctaaagcatatgcctcacctggttaaaaaagaacaaacatgtttttgtgaaagctactgaagtgccttgggaaatgagaaagttttaataagtaaaatgattttttaaatatcaaaaaaaaaaaaaaaa

SEQ ID NO: 4 - protein, 557 aa, Homo sapiens
MRDYDEVTAFLGEWGPFQRLIFFLLSASIIPNGFTGLSSVFLIATPEHRCRVPDAANLSSAWRNHTVPLRLRDGREVPHSCRRYRLATIANFSALGLEPGRDVDLGQLEQESCLDGWEFSQDVYLSTIVTEWNLVCEDDWKAPLTISLFFVGVLLGSFISGQLSDRFGRKNVLFVTMGMQTGFSFLQIFSKNFEMFVVLFVLVGMGQISNYVAAFVLGTEILGKSVRIIFSTLGVCIFYAFGYMVLPLFAYFIRDWRMLLVALTMPGVLCVALWWFIPESPRWLISQGRFEEAEVIIRKAAKANGIVVPSTIFDPSELQDLSSKKQQSHNILDLLRTWNIRMVTIMSIMLWMTISVGYFGLSLDTPNLHGDIFVNCFLSAMVEVPAYVLAWLLLQYLPRRYSMATALFLGGSVLLFMQLVPPDLYYLATVLVMVGKFGVTAAFSMVYVYTAELYPTVVRNMGVGVSSTASRLGSILSPYFVYLGAYDRFLPYILMGSLTILTAILTLFLPESFGTPLPDTIDQMLRVKGMKHRKTPSHTRMLKDGQERPTILKSTAF

SEQ ID NO: 5 - DNA, 24 bp, Homo sapiens
ctgcacggaaataactaatctgtg

SEQ ID NO: 6 - DNA, 20 bp, Homo sapiens
gagaggagctcgggttcaag

SEQ ID NO: 7 - protein, 551 aa, Homo sapiens
MRDYDEVIAFLGEWGPFQRLIFFLLSASIIPNGFNGMSVVFLAGTPEHRCRVPDAANLSSAWRNNSVPLRLRDGREVPHSCSRYRLATIANFSALGLEPGRDVDLGQLEQESCLDGWEFSQDVYLSTVVTEWNLVCEDNWKVPLTTSLFFVGVLLGSFVSGQLSDRFGRKNVLFATMAVQTGFSFLQIFSISWEMFTVLFVIVGMGQISNYVVAFILGTEILGKSVRIIFSTLGVCTFFAVGYMLLPLFAYFIRDWRMLLLALTVPGVLCVPLWWFIPESPRWLISQRRFREAEDIIQKAAKMNNTAVPAVIFDSVEELNPLKQQKAFILDLFRTRNIAIMTIMSLLLWMLTSVGYFALSLDAPNLHGDAYLNCFLSALIEIPAYITAWLLLRTLPRRYIIAAVLFWGGGVLLFIQLVPVDYYFLSIGLVMLGKFGITSAFSMLYVFTAELYPTLVRNMAVGVTSTASRVGSIIAPYFVYLGAYNRMLPYIVMGSLTVLIGIFTLFFPESLGMTLPETLEQMQKVKWFRSGKKTRDSMETEENPKVLITAF

SEQ ID NO: 8 - DNA, 614 bp, Homo sapiens
  misc_feature (184)..(184): n at 184 can be a or t or g or c
  misc_feature (323)..(323): n at 323 can be a or t or g or c
  misc_feature (506)..(506): n at 506 can be a or t or g or c
gcggcccaggcccgcaaccttccctggtcgtgcgccatatgtaaggccagccgcggcaggaccaaggcggcggtgtcagctckcgagcctaccctccgcggacggtcttgggtckcctgctgcctggcttgcctggtcggcggyrggtgccccgcgcgcacgcgcaaagcccgccgcgttcccngaccccaggccgcgctctgtgggcctctgagggcggcatgcgggactacgacgaggtgaccgccttcctgggcgagtgggggcccttccagcgcctcatcttcttcctgctcagcgccagcatcatccccaatggcttnaccggcctgtcctccgtgttcctgatagcgaccccggagcaccgctgccgggtgccggacgccgcgaacctgagcagcgcctggcgcaaccacactgtcccactgcggctgcgggacggccgcgaggtgccccacagctgccgccgctaccggctcgccaccatcgccaacttctcggcgctngggctggagccggggcgcgacgtggacctggggcagctggagcaggagagctgtctggatggctgggagttcagtcaggacgtctacctgtccaccattgtgaccggg

SEQ ID NO: 9 - DNA, 20 bp, Homo sapiens
accatcgccaacttctcggc

SEQ ID NO: 10 - DNA, 20 bp, Homo sapiens
ctttctgctgcttcagggga

SEQ ID NO: 11 - DNA, 18 bp, Homo sapiens
ccatcgccaacttctcgg

SEQ ID NO: 12 - DNA, 22 bp, Homo sapiens
aatgttgtgggactgctgcttc

SEQ ID NO: 13 - DNA, 28 bp, Homo sapiens
gaagatcttggaaggctcaggtgggagg

SEQ ID NO: 14 - DNA, 28 bp, Homo sapiens
cgacgcgtggcagcaggcgacccaagac

SEQ ID NO: 15 - DNA, 25 bp, Homo sapiens
caggcccggaaccttccctggtcgt

SEQ ID NO: 16 - DNA, 25 bp, Homo sapiens
acgaccagggaaggttccgggcctg

SEQ ID NO: 17 - DNA, 25 bp, Homo sapiens
caggcccgcaaccttccctggtcgt

SEQ ID NO: 18 - DNA, 25 bp, Homo sapiens
acgaccagggaaggttgcgggcctg

SEQ ID NO: 19 - DNA, 31 bp, Homo sapiens
tcggctggaatattcccgacctggcagccga

SEQ ID NO: 20 - DNA, 31 bp, Homo sapiens
tcggctgccaggtcgggaatattccagccga

SEQ ID NO: 21 - DNA, 39 bp, Homo sapiens
gactgtcctgattggaatcttcaccctttttttccctga

SEQ ID NO: 22 - DNA, 39 bp, Homo sapiens
tcagggaaaaaaagggtgaagattccaatcaggacagtc

SEQ ID NO: 23 - DNA, 400 bp, Homo sapiens
  misc_feature (385)..(385): n at residue 385 can be a or g or c or t
gtcccgctgccttcctaagccgascccgggctacctcggtcgtccccagcaggcgtggctggcagaggccgggcctcgccaggtccccaggacaggccccgcccgggcctcaggtgcactcccggcccgccccgcgccctcgcgtcccgccccagctccgccttcgccggcgccgctctgcctgccagcggggcgcgccttgcggcccaggcccgsaaccttccctggtcgtgcgccatatgtaaggccagccgcggcaggaccaaggcggcggtgtcagctckcgagcctaccctccgcggacggtcttgggtckcctgctgcctggcttgcctggtcggcggyrggtgccccgcgcgcacgcgcaaagcccgccgcgttcccngaccccaggccgcgc

SEQ ID NO: 24 - DNA, 18 bp, Homo sapiens
taagccgagcccgggcta

SEQ ID NO: 25 - DNA, 19 bp, Homo sapiens
ccaggcccggaaccttccc

SEQ ID NO: 26 - DNA, 32 bp, Homo sapiens
cggcggtgtcagctcgcgagcctaccctccgc

SEQ ID NO: 27 - DNA, 32 bp, Homo sapiens
ggacggtcttgggtcgcctgctgcctggcttg

SEQ ID NO: 28 - DNA, 32 bp, Homo sapiens
cctggtcggcggtaggtgccccgcgcgcacgc

SEQ ID NO: 29 - DNA, 20 bp, Homo sapiens
gccaggttaggttccctttc

SEQ ID NO: 30 - DNA, 21 bp, Homo sapiens
agcagcccaaattcttaaagg

SEQ ID NO: 31 - DNA, 20 bp, Homo sapiens
gaatcacctcgctgcttttt

SEQ ID NO: 32 - DNA, 19 bp, Homo sapiens
aaatgtggaaagggcatct

SEQ ID NO: 33 - DNA, 20 bp, Homo sapiens
attaggcggtgtcaagagca

SEQ ID NO: 34 - DNA, 20 bp, Homo sapiens
ggtcgtgcgccatatgtaag

SEQ ID NO: 35 - DNA, 20 bp, Homo sapiens
gagaggagctcgggttcaag

SEQ ID NO: 36 - DNA, 136 bp, Homo sapiens
gtgcttacaacagaatgctgccctacatcgtcatgggtagtctgactgtcctgattggaatcytcaccctttttttccctgaaagtttgggaatgactcttccagaaaccttagagcagatgcagaaagtgaaatg

SEQ ID NO: 37 - DNA, 2136 bp, Homo sapiens
  misc_feature (5)..(5): n can be a or t or g or c
  misc_feature (7)..(7): n can be a or t or g or c
  misc_feature (229)..(229): n can be a or t or g or c
  misc_feature (324)..(324): n can be a or t or g or c
  misc_feature (325)..(325): n can be a or t or g or c
  misc_feature (326)..(326): n can be a or t or g or c
  misc_feature (327)..(327): n can be a or t or g or c
  misc_feature (328)..(328): n can be a or t or g or c
  misc_feature (330)..(330): n can be a or t or g or c
  misc_feature (331)..(331): n can be a or t or g or c
  misc_feature (334)..(334): n can be a or t or g or c
  misc_feature (569)..(569): n can be a or t or g or c
  misc_feature (593)..(593): n can be a or t or g or c
  misc_feature (617)..(617): n can be a or t or g or c
  misc_feature (1063)..(1063): n can be a or t or g or c
  misc_feature (1328)..(1328): n can be a or t or g or c
  misc_feature (1767)..(1767): n can be a or t or g or c
  misc_feature (2105)..(2105): n can be a or t or g or c
ccccngnttcgcgccccaatttctaacagcctgcctgtcccccgggaacgttctaacatccttggggagcgccccagctacaagacactgtcctgagaacgctgtcatcacccgtagttgcaagtttcggagcggcagtgggaagcatgcgggactacgacgaggtgatcgccttcctgggcgagtgggggcccttccagcgcctcatcttcttcctgctcagcgccancatcatccccaatggcttcaatggtatgtcagtcgtgttcctggcggggaccccggagcaccgctgtcgagtgccggacgccgcgaacctgagcnnnnncnngcngaacaacagtgtcccgctgcggctgcgggacggccgcgaggtgccccacagctgcagccgctaccggctcgccaccatcgccaacttctcggcgctcgggctggagccggggcgcgacgtggacctggggcagctggagcaggagagctgcctggatggctgggagttcagccaggacgtctacctgtccaccgtcgtgaccgagtggaatctggtgtgtgaggacaactggaangtgcccctcaccacctccctgttnttcgtaggcgtgctcctcggctcnttcgtgtccgggcagctgtcagacaggtttggcaggaagaacgttctcttcgcaaccatggctgtacagactggcttcagcttcctgcagattttctccatcagctgggagatgttcactgtgttatttgtcatcgtgggcatgggccagatctccaactatgtggtagccttcatactaggaacagaaattcttggcaagtcagttcgtattatattctctacattaggagtgtgcacattttttgcagttggctatatgctgctgccactgtttgcttacttcatcagagactggcggatgctgctgctggcgctgacggtgccgggagtgctgtgtgtcccgctgtggtggttcattcctgaatctccccgatggctgatatcccagagaagatttagagaggctgaagatatcatccaaaaagctgcaaaaatgaacaacanagctgtaccagcagtgatatttgattctgtggaggagctaaatcccctgaagcagcagaaagctttcattctggacctgttcaggactcggaatattgccataatgaccattatgtctttgctgctatggatgctgacctcagtgggttactttgctctgtctctggatgctcctaatttacatggagatgcctacctgaactgtttcctctctgccttgattgaaattccagcttacattacagcctggctgctattgcgaacnctgcccaggcgttatatcatagctgcagtactgttctggggaggaggtgtgcttctcttcattcaactggtacctgtggattattacttcttatccattggtctggtcatgctgggaaaatttgggatcacctctgctttctccatgctgtatgtcttcactgctgagctctacccaaccctggtcaggaacatggcggtgggggtcacatccacggcctccagagtgggcagcatcattgccccctactttgtttacctcggtgcttacaacagaatgctgccctacatcgtcatgggtagtctgactgtcctgattggaatcytcaccctttttttccctgaaagtttgggaatgactcttccagaaaccttagagcagatgcagaaagtgaaatggttcagatctgggaaaaaaacaagagactcaatggagacanaagaaaatcccaaggttctaataactgcattctgaaaaaatatctaccccatttggtgaagtgaaaaacacaaaaataagaccctgtggagaaattcgttgttcccactgaaatggactgactgtaacgattgacaccaaaatgaaccttgctatcaagaaatgctcgtcatacagtaaactctggatgattcttccagataatgtccttgctttacaaaccaaccatttctagagagtctccttactcattaattcaatgaaatggattggtaagatgtcttgaaaacatgttagtcaaggactggtaaaatacatataaagattaacactcatttnccaatcatacaaatactatccaaataaaaat

SEQ ID NO: 38 - protein, 551 aa, Homo sapiens
MRDYDEVIAFLGEWGPFQRLIFFLLSASIIPNGFNGMSVVFLAGTPEHRCRVPDAANLSSAWRNNSVPLRLRDGREVPHSCSRYRLATIANFSALGLEPGRDVDLGQLEQESCLDGWEFSQDVYLSTVVTEWNLVCEDNWKVPLTTSLFFVGVLLGSFVSGQLSDRFGRKNVLFATMAVQTGFSFLQIFSISWEMFTVLFVIVGMGQISNYVVAFILGTEILGKSVRIIFSTLGVCTFFAVGYMLLPLFAYFIRDWRMLLLALTVPGVLCVPLWWFIPESPRWLISQRRFREAEDIIQKAAKMNNTAVPAVIFDSVEELNPLKQQKAFILDLFRTRNIAIMTIMSLLLWMLTSVGYFALSLDAPNLHGDAYLNCFLSALIEIPAYITAWLLLRTLPRRYIIAAVLFWGGGVLLFIQLVPVDYYFLSIGLVMLGKFGITSAFSMLYVFTAELYPTLVRNMAVGVTSTASRVGSIIAPYFVYLGAYNRMLPYIVMGSLTVLIGILTLFFPESLGMTLPETLEQMQKVKWFRSGKKTRDSMETEENPKVLITAF

SEQ ID NO: 39 - DNA, 20 bp, Homo sapiens
gtgcccagagagtcctccta

SEQ ID NO: 40 - DNA, 21 bp, Homo sapiens
ttctccctaaggcattttggt

SEQ ID NO: 41 - DNA, 26850 bp, Homo sapiens
  misc_feature (49)..(49): n at 49 can be a or g or c or t
  misc_feature (134)..(134): n at 134 can be a or g or c or t
  misc_feature (546)..(546): n at 546 can be a or g or c or t
  misc_feature (877)..(877): n at 877 can be a or g or c or t
  misc_feature (1338)..(1338): n at 1338 can be a or g or c or t
  misc_feature (1985)..(1985): n at 1985 can be a or g or c or t
  misc_feature (2124)..(2124): n at 2124 can be a or g or c or t
  misc_feature (2307)..(2307): n at 2307 can be a or g or c or t
  misc_feature (3115)..(3115): n at 3115 can be a or g or c or t
  misc_feature (3159)..(3159): n at 3159 can be a or g or c or t
  misc_feature (3191)..(3191): n at 3191can be a or g or c or t
  misc_feature (3282)..(3282): n at 3282 can be a or g or c or t
  misc_feature (3661)..(3661): n at 3661 can be a or g or c or t
  misc_feature (3748)..(3748): n at 3748 can be a or g or c or t
  misc_feature (3797)..(3797): n at 3797 can be a or g or c or t
  misc_feature (3905)..(3905): n at 3905 can be a or g or c or t
  misc_feature (4260)..(4260): n at 4260 can be a or g or c or t
  misc_feature (4903)..(4903): n at 4903 can be a or g or c or t
  misc_feature (5971)..(5971): n at 5971 can be a or g or c or t
  misc_feature (6111)..(6111): n at 6111 can be a or g or c or t
  misc_feature (6148)..(6148): n at 6148 can be a or g or c or t
  misc_feature (6400)..(6400): n at 6400 can be a or g or c or t
  misc_feature (6468)..(6468): n at 6468 can be a or g or c or t
  misc_feature (6575)..(6575): n at 6575 can be a or g or c or t
  misc_feature (7287)..(7287): n at 7287can be a or g or c or t
  misc_feature (8495)..(8495): n at 8495 can be a or g or c or t
  misc_feature (9918)..(9918): n at 9918 can be a or g or c or t
  misc_feature (9919)..(9919): n at 9919 can be a or g or c or t
  misc_feature (9924)..(9924): n at 9924 can be a or g or c or t
  misc_feature (9947)..(9947): n at 9947 can be a or g or c or t
  misc_feature (10143)..(10143): n at 10143 can be a or g or c or t
  misc_feature (10357)..(10357): n at 10357 can be a or g or c or t
  misc_feature (10379)..(10379): n at 10379 can be a or g or c or t
  misc_feature (10384)..(10384): n at 10384 can be a or g or c or t
  misc_feature (10580)..(10580): n at 10580 can be a or g or c or t
  misc_feature (10717)..(10717): n at 10717 can be a or g or c or t
  misc_feature (10718)..(10718): n at 10718 can be a or g or c or t
  misc_feature (10719)..(10719): n at 10719 can be a or g or c or t
  misc_feature (10781)..(10781): n at 10781 can be a or g or c or t
  misc_feature (11111)..(11111): n at 11111 can be a or g or c or t
  misc_feature (11150)..(11150): n at 11150 can be a or g or c or t
  misc_feature (11211)..(11211): n at 11211 can be a or g or c or t
  misc_feature (11383)..(11383): n at 11383 can be a or g or c or t
  misc_feature (11489)..(11489): n at 11489 can be a or g or c or t
  misc_feature (11502)..(11502): n at 11502 can be a or g or c or t
  misc_feature (11503)..(11503): n at 11503 can be a or g or c or t
  misc_feature (11515)..(11515): n at 11515 can be a or g or c or t
  misc_feature (11640)..(11640): n at 11640 can be a or g or c or t
  misc_feature (11937)..(11937): n at 11937 can be a or g or c or t
  misc_feature (13423)..(13423): n at 13423 can be a or g or c or t
  misc_feature (14232)..(14232): n at 14232 can be a or g or c or t
  misc_feature (14579)..(14579): n at 14579 can be a or g or c or t
  misc_feature (15503)..(15503): n at 15503 can be a or g or c or t
  misc_feature (15601)..(15601): n at 15601 can be a or g or c or t
  misc_feature (15755)..(15755): n at 15755 can be a or g or c or t
  misc_feature (15823)..(15823): n at 15823 can be a or g or c or t
  misc_feature (16062)..(16062): n at 16062 can be a or g or c or t
  misc_feature (16204)..(16204): n at 16204 can be a or g or c or t
  misc_feature (16635)..(16635): n at 16635 can be a or g or c or t
  misc_feature (16637)..(16637): n at 16637 can be a or g or c or t
  misc_feature (16724)..(16724): n at 16724 can be a or g or c or t
  misc_feature (17546)..(17546): n at 17546 can be a or g or c or t
  misc_feature (18232)..(18232): n at 18232 can be a or g or c or t
  misc_feature (18260)..(18260): n at 18260 can be a or g or c or t
  misc_feature (18414)..(18414): n at 18414 can be a or g or c or t
  misc_feature (18418)..(18418): n at 18418 can be a or g or c or t
  misc_feature (18424)..(18424): n at 18424 can be a or g or c or t
  misc_feature (20789)..(20789): n at 20789 can be a or g or c or t
  misc_feature (21317)..(21317): n at 21317 can be a or g or c or t
  misc_feature (21696)..(21696): n at 21696 can be a or g or c or t
  misc_feature (22199)..(22199): n at 22199 can be a or g or c or t
  misc_feature (23399)..(23399): n at 23399 can be a or g or c or t
  misc_feature (23712)..(23712): n at 23712 can be a or g or c or t
  misc_feature (27712)..(27712): n at 27712 can be a or g or c or t
  misc_feature (24038)..(24038): n at 24038 can be a or g or c or t
  misc_feature (24565)..(24565): n at 24565 can be a or g or c or t
  misc_feature (25073)..(25073): n at 25073 can be a or g or c or t
  misc_feature (25419)..(25419): n at 25419 can be a or g or c or t
  misc_feature (25420)..(25420): n at 25420 can be a or g or c or t
  misc_feature (25426)..(25426): n at 25426 can be a or g or c or t
taatttttttaagtctattatgctttatgcaaatgtttagatttccttnttaaaaatctgctggccaggttaggttccctttctgtagctttattttcaactcataatatagtgactattaaatattctttacncaacacattatattgtaaaaatatacacttagttctgtttctatcactaagccactactattttaaaaaggaatatcttgcattgttaagtttagcagcccaaattcttaaaggccttaggaatggtgctgtccaataagataaccaatagctacatgtggctactgagcacttaaaaaatgagtctggccaggcatagtggctcacgtctgtaattcagcattttgggaggctgaggtgggaggattgcttaaacccaggagtctgagaccagcctgggcaacacagggatgccttgtctctgtaaaataaaaaaaataaaaaaattagctgggcacggtggcatgtgcctgtagtcctagctactcagcaggctgaggcaggaggcccacttaagcttaggagttcaggnctgcggtgagctatgattgtgccactgcactctagcctgggtgacacagtgggaccgtgtctccaataaataaatgaataaataaatattaaaaaataaaaagtgtggctagtccaaattgaaatgtgctgtaaaatacacactggattcctttttttttttttttcaaatcctgagaacctgtgaaacacactgaattcttaagatttagtatgaggaggggaaaaaaagatcaatgatttacatataacaatgtaaaatatcaataatttctatattgattatatgatgaaatgatcatgttctggatatacttgattaaatataccantaaagttaatttcatcttttatttttactttttaaatatgcagcattgcccaataaaatacaggacaaccagttacatttttattttatatattcctgcttgggacataatatttaaaaattattcattctatatctgaaattcaaatttaactaggagtcctatatttttatttgctaaatctggcaattttattaatatggctactagaaaagttaacataacaaatgtagctccttatcctatggaacattgctgctttagaatcacctcgctgctttttttttttttccagttagcttctgaataaactctgcacggaaataactaatctgtggggaatatttaaccctaggctaggatcgttaatcgtgaagttaaatttttacatctgcatgaacaaaatggcttgttacagacgaaaatgtggaaagggcatctggactagaatgaaaagtcanagctcccagcctctctactagggtagttaacagtcccaggttcaaaataatccccgtccctcttataagattaggcggtgtcaagagcaactatctgtgagaccctgggccagtgactttctccttacctccgcccccaagtaggccttgcaaaaagctgggaggggtgcgttttcaacactggagcttcgcggccgctctcccactcgctccccgcccggcgctagaggagcgagttcggactcggaccccaaggcctcgagtcccgctgccttcctaagccgaccccgggctacctcggtcgtccccagcaggcgtggctggcagaggccgggcctcgccaggtccccaggacaggccccgcccgggcctcaggtgcactcccggcccgccccgcgccctcgcgtcccgccccagctccgccttcgccggcgccgctctgcctgccagcggggcgcgccttgcggcccaggcccggaaccttccctggtcgtgcgccatatgtaaggccagccgcggcaggaccaaggcggcggtgtcagctckcgagcctaccctccgcggacggtcttgggtckcctgctgcctggcttgcctggtcggcggyrggtgccccgcgcgcacgcgcaaagcccgccgcgttcccngaccccaggccgcgctctgtgggcctctgagggcggcatgcgggactacgacgaggtgaccgccttcctgggcgagtgggggcccttccagcgcctcatcttcttcctgctcagcgccagcatcatccccaatggcttnaccggcctgtcctccgtgttcctgatagcgaccccggagcaccgctgccgggtgccggacgccgcgaacctgagcagcgcctggcgcaaccacactgtcccactgcggctgcgggacggccgcgaggtgccccacagctgccgccgctaccggctcgccaccatcgccaacttctcggcgctngggctggagccggggcgcgacgtggacctggggcagctggagcaggagagctgtctggatggctgggagttcagtcaggacgtctacctgtccaccattgtgaccggggtgggtgccggcccctgctggggctgagaccagggctcggaggacctgtcgcggtccttgaacccgagctcctctctcccagatgcgcactggacgctgtcactccccctcccccaacggtcaacaccctagcgatggagaccctccagccaggtggcttgggaacgcttcacgaggtgacctccagccacagtgtgctcctccctgcacaggtggtcagtctggcctcccgtcctgatggccactttgaagagggtaccaggaaggtcctggcggtccctgggcgatgctctatggccctgtgtgtccaggacttactctagttggggttgggggtggtaagtagcagagccaggacttgggccaggggctatcccgtttttcctctagtctcttgatttctttttagaagagaagaaatacttctctttcctgaacttttaaaagttaaataaagcatgtgtatacaactgcctcttccctttttcctctagttactccttcccctaccgtccacaacccaaaaacgacaatctggtcatgccctgtaagtaattgtttgccttttcccatggtcagttgtcagtcttttttttttttttttttttttgagacagagtctccctctgtcaccaggctggagcgcagtcgtgtgttcttggctcactgcaaccttcgcagtcgtgcgttcttggctcantgcaatcttcgccttccgggctcaagtgattctcctgcctcagnctccgagtagctgtgagccacgacgcccagcnaatttttgtgtttttagtagagacggggtttcaccgtgttggccaggatggtctcgatctcttgacctcgtgatctgcctgcttctgcctncaaagctgggattacaaccgtgagccaccacgccaggccgtcagttgtcactctttaagatccattcatctgaagatgggttcagggtgacttgttgacctggaatattttctcaggtattatgaggcaaggctgtcggccagatttagttaaagcatacagccttaggtcatagggtgtgggggagcctttctcatttctcatccccttggattttccctctgggtggttttgtctgtcccctccgaacctgttggagcagttgttggagctggatgtaggaacatgatgttaatgatgtatgtgttttgtgtcttttttagacactggcactctagctccctgaagtttcagcagcattgagtaagtagccagtgantagccctcattgatagataggctcactaaatgtgcagatgaccaattcgcaggttagagaaggcttcccagaagaggaggcctctcngtgagcctagggtgttcagctcagcataatagtgagctgaaggccccantgcagcagcaagaaaccacccagcaggacggcggagttcacagagaggggagagttcatagagagggagagtgccacaggccctggcacagcttcaagccctgctggntgttggtgctgagcctcccctcctggagcctcagaggggcttacaggggctctggagatcccaactgtgttgcttcttggcgtcatcacccttcaatggagtctgagagctaccctgggagatccaagtgtgtgtgcatgtgtgtgttttttctttttggaaatttgatgtccccagcatttggacctgctttctccacatatatgtagtgggagtgtgaccggagccccactgggatttcttagctagtgacatagcttcagtgtccagaggcatcatggcttgacaaaagaggcactccttggaggtagccaactgtgctttctggcccatgaatccacaggaaattggaacnttcagttgccacaaatcctgggtcctagcctcacaggtgggtgtgttaccttcccggaagccagtgagtactatgaaagcagaggctgtccctgaggttgcaggcagaggccacagaggggaacatgacacaggaatccctacaaattctacttggggctgcctaaagaagagggaagtagtgaagcaagaagaagcacatggcatctcttggagttttacattgacccctgagggttccccggcttactctagtcacttgttcctgctttgctgcctccatcccacattgggctgagtgatggtggcattgatgagctcccaaaggccagctgtgccagggggtctgaccttatcttgctgccaatgtcagccttttgttttttaatatttagactatttatttagctgtcttagcagtttcaaaggagttatgtgccctttcacctacttatatgttgtcagtctttgcagggaggccagattaatgcttagatctttgttttgggctactggaatgcttgacttgaagttcagagctgcttgttcccaggtgaacagctactgctggaagttgctgcatcaacattctaatggctttttctatggcctgttgtctttcaacccaaacctggcctgctgancactgcatctagtcccatgcctgctaaatgtctctaagcctgccctctgccccaaatcatacataaaggtgtttgtaagtacactggtattgaattactagtcatattttttccactgaagactggaacctcaggtgtcctgtttggatttttttaatttgttcaagttaaagtacatacatgtagtaccaaactttgtggtggatttagatctttgcgtcttcctcaatttctgacacaattctggatgcagagggaggttctcaggaaaatttttattgaatgagttaatgaataatttaagaaatcatctctaaagtttgagaactaaagaaaaatagttcagttcttagaagggaaacttgagggtggctgaaaaggattgactggaattttttaaaggaaatgtgactccccctgcccactgactggggctttgatgccacatggatgtggaatgaggtgttgggattggcagagggaatctgctagcaattaataaataaataaatattggcagggcgtggtggctcacgcctgtaatcccagcactttgggaagccaaggcagcaggtcacttgaggtcgggagttcaagaccagtctggccaacatggtgaaactccatctctactaaaaatacaaaaattagctgggtgtggtggcacatgcctgtattcccagctacttgggagactgaagcaggagaatcgcttgaacccaggaggcgggctttgcagtgaaccgagatcgagcctctgcactctagcctgggcgacaaagcgagactccatctcaaaagaataataataatattaataaataaaaatgatttatgaggtaaaagagttttatgcccccatgttccaggaatagtttggtggtccacatggttctcggctggcctctcctctggcccctcagtcatccctggggtactggggaattagccaacccatcatgcagtgcttcttggccatggactgccccatctgctggaaacctgggttgtttctgaggttgtctgggctgtccgctctttggtttcaccatagctctgtccagcctntggacagacaggttccttgagaaacttccggctgggtgtggtggctcatgcctgtaaacccagcactttgggaggctgaggcaggtggatcacaaggcaggagttcgagaccagcctggccaacatggtgaaacctcgtntccgctaaaaatacaaaaattagccctgcgtggtggngggtgcctgtaatcccagctactcgggagggaggctgaggcaggagaatttcttgaacctgggaagtgggggttgcagtagccaagatcgcaccattgcatgccagcctgggtgacaagagcaagactccgtctcaaaaaaaaaaaaagaaaaagaaaagaaacttccaagctgctctgcatcgccttgctctccacctgtctgcttctaagaagccctcggcccagtcctgggtgggactcccactccctncccattgtcctggactagcttttctatcagccttatcttgtgtagagacagatagtcttagaagatgngagagccctcactgttatccccaaagctgcctggaggaaaagccagagcaacctgggagctgggaccggggctgactctgggcagcagagacccgagagacctggancttgaacctcactgttacgcctttgttgatttctctcactcaggggacacacagaccctcatccagccttttgcagctatatggcaaggcagagaagccacttgcggggtcccgtggcccactatgcacgtacatagtagacacatctggccatgagtggtcagattgagccactctctagctagctgacacctgtcatcctgggtcaaatttctgacagttgacacaaagcagggggtcagggagccaaaaaaaaaaatggccaggtgtggtggctcttgcctgtaatcccagggctttgggaggccaaggtgggcagataacctgaggtcagagttcaagaccagcctggtcaacatggcaaaaccccgtctctactaaaaatacaaaaattagccaggctggtggtgcatccctgtagtcccagccactcaggaggctgaggcatgagaatcacttgaacccgggaggtggaggttgcagtgagccgagattgtgccactgcactccagcctgggagacagagcaagactctgtttcaaaagaaaaaaaagagtattctggagattgaagttcaggagttcagggttcatctcgactttgggcagccaagcaagaactaaagtataccaagatgttgaaggttgatacctttttatttatcgattcattcactcacctatgtaccaaagagctcctgagcctctcttcnatacagggggcactgccaggagttgtagaggatgtgatagcaaagataggaaataccttttctctttgctctgacaacggtggggcaaggattcatcattgatttcagcaggaggcaggataaaatgtgtgtaggaatataggaatacatggcaatcagtaacatgtggtacctagcagcatgtctgactgttgatacggtcaggctaggtacatcccctcagggaagaacttctgtcttaggggcacacaccctatcttttttcccttcctgccaattcacaggtaagaacatttagtcccagggaactatgtcatctctctacttctcataactgaaaaagcagtgccaattatgtatgaggtataggagacacaattctccctctttttaaaaatgtttaatagctttattaaggtgtaaatgacataaaaactgcatgtagctaaagcatgcaatttggcacacatgtatctctacacccttgaaaccatcaccactagaaaggtgcatttctccccaggaagaggggcaagtctaggccctttgccagagttgctcccagattgttttcaggttgggcctgcattcacagctcagccagctgaagggtgacagcattagactcgtgacccaaatcttaaacccacacattccattttaacattgatacctgtgatcatcagccagtatagcccatcccatgtgccaggcggaggcatcaataagctggtctgtagcagctttaatcacagctggggagccaggagctaaggaatgctagactcctttgtaaacaatttaagtagggggtacttagcctggactctatgtgcttctcatccacctcttctcagggagattctggccaagtcctggaacctactgcagtccatctggtggttgacagatatgtggacggattggcaggctgggacccaatctatgtttgcccttgtgttcagttttgagacctagcacctttcctgatcctgctcacagaccccctgcggccaataggaaagaagtgttaatgcatatttgcttttggagggcccaaagccaggcccagagagttgtcaagggcggtcagtggtgggtggatggcagagttaaccaaggagttacacacctgcctagactaaggacaggctggaccaagtagagagggtggggctaagggagcctgagagatgctctggggcctntctcaaaatgagcactatagtcaccctgtcccctgcagagattgtctgacctggttttaggtcacacccaaccttgccagccaaggagtctttagaagcctgatattgggagacctgtcctggggtctacaaccccagaactcactgcagaagcccacgtggattgctagtctagctcagccatatgggtccccaaccctcacctcatgatagtcctgtgagaaaccgctgctgaccctttgttcatgttttcatcttttccactataaaagacatgctagctgggaaatagagcccatcatactcaagagtggcaggagccaggtcctggcccctgaagcttggcctcacacacagaggccggcaccctgtcatcaattccctcagctttttctccgcctccactcccagtcctagatttagcagccatgtgtgggtgggggccactgcagggatacttacccacctaccagagagatggcctgtgggtgctggcccttctgaggctgtggaggttggaggctgtggcagcctgggcagtcaggctgtggtcctcccatgttcttgactcctgctagtctgggctgcctcctgattaggggttggatgctccagttcttccctgggttggggattgccaccctactcccagcccatccaggttcacgcttattccaaagcggagcaccagcagtgtgcctgctgcgggagttctccgtgtccagcctgagggttgcctgccaacccctctgagaggtgcccggaggctgtgcgcccacactgcccagcagtgcggagaagcaggcttgtttttccctgtcactggcttggaagagatgctttgttctagggagccgcatgtccccttgcctgcgttgttggtgaggagccagcaggctccgtggagggcaggctagcagcctggcaccagggaggcaagggtctgagttcctaggagggtggttgctcatgtgagaagtctgcaaaggttactactgagcaccatctctctgtctgaaaaacatttttcatttttctgtgaaccactaagtttcccgtttgggctttcttcctgcttttggctcttgtttaggcaggcgtagccagatccaagcgtctggctgcttccccatgtcttcagacctctttttctgttcatggtaactatagatggaaccacacattggaagctggaaactcaagcggtgcagcctattccttaccccaatccctgttttacaaatggggaaatcaaggcacagcatggggtgatgcttatctgaggttggaagagttaatagtagagtgggagctaaaacccagtttcttacctccaagctcagggctttcagctgtaattgagcctagtatagttggtgtgcagcatcagggattccagctctaaaggtcacaaaaaggaccnnggggntcattggcccagggtgggaaccngagcagagcaggtccagatggtgcactctgtgccctggccttagtttcttgctggatgctttggcccgtagagccccagagccctgcttccagaaccactccagtgacgttcatgccaatggcctgaaccccactgagcgagggtgccctgcctcttccacagccctgggctccgctcagatttttaggagcaagngttagaggccttgctttctccagggtcagcatgtggacagaacacttactctctgcctgtctctcctcctcaaaatggaagcaagacagtggggcctacaatgctatgaaaaacaggatgggaaagaagcctgctctctgccttcctgcccaggtgagccatcacctgactaagtgagttcacactcagagcgtgtggggatggcaggatgttntgacttcattttccaggatgcntttgntttaaaaccttttaaaaagaagtgaatgatacaccccctttgctcatcttgcagtggaacctggtgtgtgaggacgactggaaggccccactcacaatctccttgttcttcgtgggtgtgctgttgggctccttcatttcagggcagctgtcagacaggtaaggtgtctgtcttctggagcaccaggggacctcancactgaggaagaagcgtgtgcctggcccttgatttcagttggtagtattctgtcagcgcagggccctgtattttaaagaagaggaagctatgtctgtgatatagactccatgcctagtaagaagagccaacaaatcnnntgactccgtaattcttgctaagtaaagaaacctgagctgtctaagctgaatgtatctgtganccggttgactaggtaatatgccatgattcacttctgcagtagcctggcttgcctcccctgggtcactgtgactctgtcatgcccctgagcatgggagaggttgacatcatgcacacatgcacatgtgctagattgtagatctgtagtagtgccacggtgtctgcctctgtagtcccaagaagaccagcattctctctgcaaagtgaaaggagctctcaccagccactagtggtatgaaaagcagaactcttttgtccacaaggctgatgccccttagctaagtggcctgtggttttggcatttactttatgacaggagggagaatagtgntttgatccatttcttataagcaggttatttgtataattntaaagcttttaactcaaggaaacattaacggcttagagaatcccaaacccctcgaaattanatgcacaatgttagagactacatgtgagcatttttttggagagaggtccgtagctttcatgaagttcttagaggggtccattatgttgtgtcttctttccctggagcttcagggtctgtcagagaagactgtgaagagagtagcagccttcagcaagtccttggccacatgncacatgtgagaacaccccacaaatcggtgggttagccggtggaaaggagtcccagcatcttccctggtttttaattcctggcctcaagcaatcctccctccataanctcccaaagtgcnnggattacaggcntgagacaccatgcgcagccagatattttttattgttgttgtttttttttggaaaaggagtttcactcttgttacccaggctggagtgcaatggcacgatctcggctcactgcaacctctgcctcncaggttcaagagattctcctgtctcagcctcctgagttgctgggattacaggcatgcaccaccatgcccagctaattttgtatttttaatagagacaaggtttctccatgttggtcaggctggtctcaaactcccgacctcaggtgatccacccacctcggcctcccaaagtgctgggattacaggcgcgagccactgcacctgcccagccagatttttttttaaaaagcaggttaacctgtttattattcctactttacagatggagaaagtgagacagagggattaaataacttncccaaggtttcacagctggcaagtggcagagttaggatttggacccaggtagtcttgctcctctattgtgtatggactactgttctaggtccctgctgtcctaaaacttgctttctagcaaggtggaatgtattaaacaaccaagtgaggaagtcgttgttgtccttggccatggtaagagatacagagaagtgcagggtgccacaggagtgtctaacagaggaggtcaggggcagcatccctgagaaggtgaagcatgagcgagagtgggaagatgagtcgaaagtagccagctgaggggtagagaggagaaagaacatccaggcagggagaatagcaagtgctaaagccggggctcatgaaaaggcatgggagcaggacaaagtccgtgtggtagaggtgcggagagtggtgttaagatgaaggggagaggcaggcagagccctgggcagatgagcaaccagggcttagtggatcacagttaggactttgggcttcagccacagagcagcggtgggctactgagattttttaaagcaatagtgtgacaatcagatttgtcctttttttttttttaaagatgctttgacaaccttgtgaagaaagaattgaagggaagcaaaaggtgttgtagagagaccaattaaaaggttgtcacagtagttcatgccagagatgatggtggcatggcctagcatgacggtggtagaaatggagggaagtggtaagaggtaaaatcaacaagacttgccgatgggctggatgtcggaattgggggaagaaagctttctggcctgagtaactgggtgaatgaaggtacacttctctaagacagagaatgctggaaaagaaccacgttcatggatattgagttcaatttgtgtgtactaaatttggggtgactatgagacccctaagtggagaagtagagtgtgaagctggctgtatggatatggtactgcatgagaggttttggctagagaaacacatgtaggatttgtcagcatatagaaccctcagcagagccccagatagggatgaggttgcctggggagaggggagtggggaggggaggggggaactgggggaggattgtgctatgcttagaggccacctgagtggacatgggagattccttctcacggagctaagtgacagctgcggctcagaagaatgcccttgatgtgaaaaggagataccccaagttcagagtagaaatgcagtgtatttttctgggtcagctgtgtcatgggtcagcttaagaaccttcttgccatgtgacacaatgattacctgaggaaagcatttaagttccaaaaggtctattcccagggaaagtggaaacaaaatttgtgagtgtgtttaggaccactttagtctacagaagggctaatatagtgtttttcaaacctttctaaacattttggccacagaactttcattaaagtaggataatttaagtctagtaaatgaaatacaccctaantggctaaaagtatggctgttctgcctgcagcccctgccttcaattcccaatgccctgcctcaagcctgtctgtgcccccttggaaggcccagggccctgtgggatggacagcttctgagtgcattacttctgagtaaaccagttctaaaacctaagctaagtagattacctgaaagcaatcaattcatgccctaacttgtctaggacatcaatgtaactttttatatagtataaaggtttcaccttctttctggcctatgaatatgtctgaaattcaagagcaattaaagtactcctgggttgtcaaagccctttatgtaacactattgagttatctttatgcgtctgacttgtaagagatgcacaactctaggaagaagtagactgctgtgtcctgtttccaggtatgtgtgtgtttgccattttgttgacagactttaaaagcaaacatttctggccccaaccctgaactgccaaggactggtgctatgtaaagggttctctggatctgtctcttccctaccatcccagggagctcttaggaagggaaagggcatagagattataccagcctgccttgtggttaggaaccaccccttggttggcatatagaacatgcttgttaaaaaaaccatgcaggggaaagtagagtctactaccaggcgagagtttctcaacctcgaccctattaacattttgagccaaataattttgttctagggcattttcctgggcattttagaatatttaacaacatatctggcctctactcattagatgccagtgaaccccaagtgatggaaaaaaanaacaacaacagaaaaaaacctcttttattgaggaaaaacaccaaactcttccacatagttgcaagaccttgtgcaatttgcctcctagccaccactgtactcttgaattgcacgcctgatgccaaccacactggttcctcatgttcaccatgccccctccagccatgggggtgtgtggtcttctcagagtctgaagcattccccacccaccccaacccaccccctgtggccttctttaaccatgctggctaattcaggatccctagttccttatgactttcctttaaaacgtctaccagaaattgggggaaaaaaagtgttattataggattaatgttggtcttccncactatactgtgaatatcattgagagcttggtccctacaccttaaatcccccatcgtcaactattttttcccatctcagtgtcccatgatcaaggagaccctccctgaatgtccagttccccaacccttacccccagtccagggtagcttccttccttgtgcctctcattaacctgcatgccgatccttcagtgcacttgactcagtgtgtaattgtatattcagtagcgtgttgttagattaaaatgtggttaatatgtgtttcaccagttatactatgacactccttaagggcagaaacagcatcttttttaatttattgatatccaagtgccctctataatagatgctcaataaacattgaatgaaagtgggtgtcagccagtactggccagactcaaactgaacccactgcttcccactagcttgactttttcctcctgtttgtggcactctctttaaaacaaaccaaaataaacccaattttaaaaactttttaaaatgagcacggatacagaaaaccacacagaacaaatgtgtagcttaatgaatttttttcagagaaataaccttatgaccaccaccaagtcgagcagtagaactttgctgtccactaagaagccctgtccatgtgccccatcccaattacagcatcctctctctctccccattaagtaaccgctagcctgactcctgtaataatcacttccttgtgagtttttttagttttattatcgaaatatgcatccttgacacaaatttagtgttgcccacttaatatatttgatgtcttttagtctacttaatctatggattctccttctatcgccttctatgccttactgattatctatgaagaacctgagctattccacctatagaatttcccagtctggatttgttgattgcacactgatgatgcanttcagcacattcctctatgctctgcatttcctcaaaattggcagttggatccagagacttgagattcaggttctgattcaggttcagtccttttggcnagaccataggaagcatgcaattcctgactgtctctttatgatgttaacagtaattagtatataatgcatagatctattaatccattgggggctataaatggtattattctaattttattaccttttcatttaaaagttagaatacttttgtacntgatactacctcttatctattattggttgctgttcacatagtttacaaaggaaaatcaggacaaatgnttctttctctttatttgccagttttcatttataatgaattgtttctctgttattctccaaatttggcagattcttttttaaaaaaaatatcattatgaatgtatggattaaataattgatgtatttcagtctcttgcaatcattatcataattgtagcattgctttttaggcaaccctggtacccaggctgtacatttgtcatggggagtggggagggggagaaatagcatgggcactntgagaccgagactgtccctggcagccagtattctggcaacactgttcacacccacttactggatggatcttgagaaagccccacttggtggagcccattcctgctgcccttttccagctggttatctgtcactctccttttnttcccaggtttggccggaagaatgtgctgttcgtgaccatgggcatgcagacaggcttcagcttcctgcagatcttctcgaagaattttgagatgtttgtcgtgctgtttgtccttgtaggcatgggccagatctccaactatgtggcagcatttgtcctgggtatggccatcaggttggagttgagtacttgatcctgtatttcaccatcatcccatcacctacctttctggagacaactgtgatgtccctcaagggggacagggtttctaacaaaactagccagagcttcctggtgaaccttacttacaggcagggaaactgagccagacatgagaccagcctgggtccccagcagcacaatggcctgacttctgatttccagttcttttctggcctctgggctgtggctccttggtcttagtacttgntngtcaatttactaggactcaccagagatcctccatttacaaaaagggcctgccactgcacagggctgagccagccccagaaagagggnggcatggttggaggaggaggggctgtgactggcaagcttgctaaggtagagaaccccttgtctgcagagcactgtggctggtgatatctacggacaagaataaattgataggaaggggctttcgtcaccttcagggttttaattcagagtgcacactgcagggctttgtctcaaatgtgccagcctgttgtcactgagaagctgccaggccggcctgtgtttggaggaacctgactctagctgataaggcctttgagttccttgggttgtattgttgaaagggttgttttttctttttatatttaatattctttccttgaggcttaagtcagcatgtgctgacttagtaatgacttcacttttaataaattcttcctcatgtgaggattaaagggggcctaccatggcatctttagcacatggcttcagaacatggcgaaattttcaagagagaactgttgcttgggggcctgagaggccacagggatgtacccccaggagacagtcagacaggaggggttcagaacgccatccgctccctagcgccatgaacttagagagagttctcgctgttttcttgtctgtgtattcacaaagataccataaaaaattaataaggaaggaacccaaattaaactgctaactcgacctcccttgttttgaacagggacagaaattcttggcaagtcagttcgtataatattctctacgttaggagtgtgcatattttatgcatttggctacatggtgctgccactgtttgcttacttcatccgagactggcggatgctgctggtggcgctgacgatgccgggggtgctntgcgtggcactctggtggtgagtgtgaccctgtgccccatgtgcccactggcaggatgatttctgtctggccttcactagagggcagcaacaacccatgaatccctattttgtctcccagagacaggaagcatagattataaattatttcagaatgttttctccacactcaaaagagccaaaacaaaacagaatcccatgacagcaacagacttgctctcagccctgtgctgggttgccccaagtgtggggaaaaatagcagtagctgtgagaagatggggtccagcatgccctgtaggaagttcccaagcctcagggcaggacagtgtaggccctagttctggctgtgtgctgctgaagcctcatgccacaggcactggcaccaaaagcaagagtcctcagggtagccacatggaggaagccaggctccttctgcaccaccaaggtagaggagttgaacaggcagagaagaggccattccagaccaagaggggaacactgcagaggtgctaaggtgggaatcaccccttgcaggtggagaaggtgagatcaccagcccaagtggagcagagagcatttcagggcatagtgggagagtaagccgcacatcatggggcccagtcatgaccgagggtggggggcggctacctggtcccagcaaggtggaaaataatatccatagagcnctcaagtgccttgataaacatgctaatntttttcctttttttcttttctttttctttttttttcttttttttttctcagacaaagtctctgttgcccaggctgtagtgcagtggcgtgatttccacttactgcaacctccgcctccccagttcaagcgattctcatgtctcagcctcccgantagntgggantacaggtgcctgccaccgcacttggctaattttttttgtatttctttggagagacgggtttcactgcgttggccaggctggtctggaactcctagcctcaaatgatccaccggccttggcctcctaaagtgctgggattataggagtgaaccactgcacctggccaaacatgctattttaggtagagtatctgactaatctgttggataaatcaggggtagggtgaggagagaagagaagctaaaaggccagtgcagaagcttctgttggtgccggggacagggaggagagtgtagcagggcctgggctgacatagacatgcacagaagccaggcttccggagcccatcttgcacccatctcctcagcccagcagatggcaacactgctcttcagaaatggaggtggccagccagcatggggatgccgtcagggggtgcagggctctcccatttttgtgcggtgtggggtacacataagctcatccaccccaggttattgctgcgtgtggatcagctctttgcttctggcttgtgatcaccaaacattccacaagctctggttctgcaaccttattcccacctatggctgtgctctacctggtctgtgggtctgctgttggcagggaggcctcactgagattggaccttgtactgccaggttcatccctgagtccccccgatggctcatctctcagggacgatttgaagaggcagaggtgatcatccgcaaggctgccaaagccaatgggattgttgtgccttccactatctttgacccgagtgaggtaagcaccatgtgggtgtgggtgagagggacagactgaccgtgatttgagagcagcagcacccagccctgaagtcctccctgctcacagcagcccagccctctctctgcccaagccccaactgcccattccccccatccccccactccccacccccacacgggccctgttaacactcagaagttgaggaataggttacagctgcctcactcttttcaccacgggtttcagattttcattttttacttcctttctaggcaatcatatattttaaccattacttctaacaataaatactctttttgagtaataggcctttcataaagtcagcatttgggaaaatcattgtttcttatacctaaggtggcttgtcaccttacaaagctaaccccaaacgtaaaatgtaaagcacaaatagatttggagttagaagtatttcatctcttgagtattagcaattattcattaaaaagaaaaaaaaagtgtttagtctctttctgccctccaatggttaattattgcatatcatcttggagtcaggtcctttttgatgtccacctcttccccctaccccacccctcccgtcagccctgttctcacacaccatgactcatttcttggctctacctagttcctggttcttgcttttccttcccgttccctctcctaccatctctgtagcaggcagttttccttggtctcgtgactatgagaggttagaagctgtaaatgctgcctggtggggttctggaatgtgtctgtggtctgactggaagatgaggggttgggtgtgggaacagccacaagcagccctgctgaagtgtgagaggcaggcatggttgggcttggaaaagagggaacagttattgtagacagcggaggccaatggccactgccagccctgcagacttcccagtgagtggtggcccagcagccactgtcagcatgcaccagaaaggggtcctgtgcgcaaaggtcaggcaggagtgtggcagagggcttttaagttaggtggttttgggggcttttaagtgaggggtcaatctgggtgaatgcataagccccactggcatctttgaggaaatgaggctatttcaggggatactttcagtccaaagttgaccttttgttgaacttctaactctggaaaaacaagctccaaacctgggtttgcttaagaaagcaacatcagtgtgtttagacgtgtggtttattaatggccttggctgtgctgaatttcataggaagtcactctgggtgaagctcaggtcaattttcctgtttttctatttgaattctttttccctggaagcacaccagtaactacatagtataaggactcaaaacattaacttttaaaaaatatcagaccaataaaccacacagccaggtactctctctgacccagagggcagggagccaggcttcgggaggaatacttagaggcctccttggaatgtggccaccgacaggaatatgtgggggtgcagtgaggaagctgtcagcctgggcctctgtcttcctgtacccttgagggactggtcacttacttttcctcattttcattcactctgatttnttactgacaaggcctagggaagttttcacagcctaaaacacagtcagtatacttactgttcttagaaacgtaacactccccgacgctgagatgcagacagctaagatgccagggattcaagtatgttattgtgtgctctgagtctctgaccacctcttcttcccatacacttatgatgttgttcctgcagttacaagacctaagttccaagaagcagcagtcccacaacattctggatctgcttcgaacctggaatatccggatggtcaccatcatgtccataatgctgtggtatgtaaaagagacctgcctgaggcttccagacaaagcttcttgaagtggccattgggcctcttgtttacagacatgcctcagacaaaattcaaagcctatgtcatcagagagtgaaaaggatatgtcttgtgttagatggaaaaaatgggcatgtcacaattcttaatgggatggaacctcagaaaaggagaatgaaaacaattgtggaggctgttgtgggaaatatggactctngtggggaatctctccagatcttaagatgaatccttgcccaatttgggtcatttagttcccgtctcctacccagttaccgacagtggctgaggaggccaggtagggcttttaagaaggatctgagtgaagacaccatgtcctgtaggctgcagaggctgccagttactttctggaaatgtggaagtgggatgtgctcctcctgggatgtccataaacggtcctggagtcagggctatagcctagatgtccttaccaggttcccactaatgaggcaaagtatgtcagaaagggatttgtgaattaccagggagaggaaacatgtccaagtgcacatcgctagcttttgctcagcggccgaaccctgggattctaggcgacntctggagcctggtgggttagcggtgagaagatgggcgaggagggcggacttcatctcagagtccttattactagtctcatccagctttgaggcagtcagccactgtgcctactgagggagtgctatgagtcacccgcttccaaggaatggcccaggatccctccaggcagttcaccattccctgagttggcctcaagacaggagcagcatgtagcctgcaccacagacatgcaagcctgtgatgagtcacccacttttgtgttcacccaggctctcctccctgctctggatttcctggggactcatgcacatactctttttattgtaccagctgtgtgttccacctgcagatgagtcaaaacagtctaatccataaaggtctggtttgtcaaagagtgtgggtcatcaacagagagaatgcctactggggatgcccaggtcaggggtactgcagggcatcctgatgagaggcagtgtggcccctccattgggagccacctctntgctccacaagtaccgcggggctggtgtcagctgtctctgaccagcctcttcctgactggtcaccacaggtagtgtgtgagggtctctctccaagtgtttgacctaatgttgttccttttgttatcttatcccccaaatcctatcacacctcacttgatgtctgcctcctgactcattctctagctccttctgcagttgctggatttgaggaggttcagcttaggatttttaaagctgaaaggcaggttggaatttttcttttcaatgaagtaaatctatctgaattatacaagcttttttgctgggacactgtctatatggaaggctctgagagcgcactggcgcagggtttacactgtaccacttgggctggggaaaattatcttttgatctatgaagtaagacgcagggttacagttactgctgccttactagtctctgcttaaagatggtttggaatttactgaaataattgcattgtaaaagttgtacaggttgggaaagatgtggatactgcttttccagctttcttctgcactctgtttcaggatgaccatatcagtgggctattttgggtttcgcttgatactcctaacttgcatggggacatctttgtgaactgcttcctttcagcgatggttgaagtcccagcatatgtgttggcctggctgctgctgcaatatttgccccggcgctattccatggccactgccctcttcctgggtggcagtgtccttctcttcatgcagctggtacccccaggtagggaccatgtgcatctatggtttggggtcttcactgagtctcttactgtctaccaggctgtctcaattaataaagagaataaaatcaagcccatcacagctcccttgcttatatacattcttggcctaaaaatcaatagaaagtgtcttctgagactagaacacttatggcctgggctttgagggagtgggaaaaagcagccattggggctgttggttaattttactctgtacccaagttaatgtgctcatactgttttccactgcagaagaagagggaagaaatagctatcccattcctttttttcctggccctgtcttctttatttattcaacaaatagctatggcatgcctaccatgggctgggcactgtgctcggtgataacgatacacaagaaaacaagccaggcagaaccccaggccctcatggaacntacaccctagatgagaagacagacaacaaacaagtaaataaaatgcttaatatagttcagactgtgttaccttctaggaatacaaatgaaggacaatgccgagttagtttacatagtcacagatagtgtccctgaacagggggcagttcagtagaaatgtacataaagtgacagaaagccctgaaaaagtctaggagaacattttaggaagaagaaatggcaaaggcagcgaccctgagcaggggatgagcctggcatgtttgaggaggagggagaaggggaggggccagaccactgagagggcctcacaganccttagcaggattttatttctgaaactatcttagtatcccacagatgggtgggaggtagccatttccaataatttatagaacagttcatgggccctcatctctccctctccatcactgtgcccagagacttcagtgtacctgtagatttgggagcctctgatggtcacttttgggcccatcaggctgagaacactgcacgggaacagctccccatgggatgtggcaggaggagcccagaactgatgtagaggctcacagctgagctcagagtgaccttcaggtcacacatagctctcccatcagcacagcacagagagattagaanatcaactcgagattctgatggcctatgatttttttgaggtctgagtgggaggaaagcatgaaatgagttagaactgaattctccattcatctaaacatcatgagttaattccatagtgcctgcagtgtgaggttctggggtgacagttaatccctgacagacatgtctttaatgacttatagactgggaagcaggttgattggactattaaggagcttactctggtggtctccaggttgaggaaagtgcatgtccttatagctgcaggtcccagcctcctttcagcaatcaatttggagggaaatcttggctatagccccttcccccacaataggaagtgatagaaactgactccccaaaaaatttgggaagaaagtatgtttgttttgctctcaatagctgcatgccatgggttggtacctactcctaccctctttcctttgcttctccagacttgtattatttggctacagtcctggtgatggtgggcaagtttggagtcacggctgccttttccatggtctacntgtacacagccgagctgtatcccacagtggtgagaaacatgggtgtgggagtcagctccacagcatcccgcctgggcagcatcctgtctccctacttcgtttaccttggtaagtcccatgagccaagggcacactagagcaacgggatggaagtactaactggcttggagctggaggttgcgtgttaacaggaaaacaagttcatacagtacatgggctccatccagtactggatctttggccgggaagggttcttgtcccagtgcactggccctcactttcaaatggaaaacaacctatagattacctagaaattgatgagaatattagagggtttgtttctgttttagccatcccaggccttccatcagagactacaattcctttatcctaagaacctacagagtggtttagggagccagtgtgcttagttggagaaatttcttggaatcagagtttaaaaggaacatgaggggaaagatgtccatgcaagaggtctgatgaacgnaaaattattataacctagagcactatagagtgattttatcttgtgtgaagatccaccccatgccattttatgtagcaggtctccagttttctcttctcagaattatgtcttcatagcacctgtggtttccctgcacatccctagccagtacctctttagggagggtggcacccacctgagagtactcagagtgctttgtgaacatgctatgtagatctcaaagcaagcaaaagcaccctgcctaatctgaaggcagatcacatgggctgggacacatctgcagaggtggaagagttatttccatccctggacaagtacctcaggttccttggaaacccaaccttgnnaaatangaataatcagcatggcagaaataggaataatcagcatggcccagctcttctcctgcaaccgcccctttgtactcctcccctgcatggtggaacactgctgggctctgggcatgcctgtgccagctctgggttctgaaacctgtctagatgccagattctaatctgactgctcagactgtgagagatgtgagaccaagaaggaaagtgatccccttccagagtcctgggagcataaaggggtagatgagagaccaagtctaactgcagccctgggcctgaggctccgtctgctttgccataggtgcctacgaccgcttcctgccctacattctcatgggaagtctgaccatcctgacagccatcctcaccttgtttctcccagagagcttcggtaccccactcccagacaccattgaccagatgctaagagtcaaagggtaagaagacctcctctgtcagtgttgatgcactgggtctgggtctggccaggtctcaggagcccctcacaatagagctactcgcaaactccctctcacagacaccatggactagtttagccattaaagggttgtaaatggcaaggtgcttacttatagcccatcctctctggtctgttcctgtgtggacatgtcactatacacatctccatggcagtagccgcactggataactcagaggctagaagaaacctttcagaatctgctgcaggattctcttcccagggaagatatcctcagttcttgtttgtttggagactgggaggcatctttttaaaatgtgttactgacatatttttgcttgtttttatagaatgaaacacagaaaaactccaagtcacacaaggatgttaaaagatggtcaagaaaggcccacaatccttaaaagcacagccttctaacatcgcttccagtaagggagaaactgaagaggaaagactgtcttgccagaaatggccagcttgtgcagactccgagtccttcagtgacaaaggcctttgctgtttgccctcttgacctgtgtctgacttgctcctggatgggcacccacactcagaggctacatatggccctagagcaccaccttcctctagggacactggggctacctacagacaacttcatctaagtcctaactattacaatgatggactcagcacctccaaagcagttaatttttcactagaaccagtgagatctggaggaatgtgagaagcatatgctaaatgtacattttaattttagactacttgaaaaggcccctaataaggctagaggtctaagtcccccacccctttccccactcccctctagtggtgaactttagaggaaaaggaagtaattgcacaaggagtttgattcttaccttttctcagttacagaggacattaactggatcattgcttccccagggcaggagagcgcaga

SEQ ID NO: 42 - DNA, 54550 bp, Homo sapiens
  misc_feature (351)..(351): n can be a or t or g or c
  misc_feature (436)..(436): n can be a or t or g or c
  misc_feature (935)..(935): n can be a or t or g or c
  misc_feature (1725)..(1725): n can be a or t or g or c
  misc_feature (1874)..(1874): n can be a or t or g or c
  misc_feature (2031)..(2031): n can be a or t or g or c
  misc_feature (2152)..(2152): n can be a or t or g or c
  misc_feature (2297)..(2297): n can be a or t or g or c
  misc_feature (2747)..(2747): n can be a or t or g or c
  misc_feature (3477)..(3477): n can be a or t or g or c
  misc_feature (3535)..(3535): n can be a or t or g or c
  misc_feature (4035)..(4035): n can be a or t or g or c
  misc_feature (4084)..(4084): n can be a or t or g or c
  misc_feature (4087)..(4087): n can be a or t or g or c
  misc_feature (4321)..(4321): n can be a or t or g or c
  misc_feature (4374)..(4374): n can be a or t or g or c
  misc_feature (4394)..(4394): n can be a or t or g or c
  misc_feature (4429)..(4429): n can be a or t or g or c
  misc_feature (4480)..(4480): n can be a or t or g or c
  misc_feature (4482)..(4482): n can be a or t or g or c
  misc_feature (4704)..(4704): n can be a or t or g or c
  misc_feature (4799)..(4799): n can be a or t or g or c
  misc_feature (4800)..(4800): n can be a or t or g or c
  misc_feature (4801)..(4801): n can be a or t or g or c
  misc_feature (4802)..(4802): n can be a or t or g or c
  misc_feature (4803)..(4803): n can be a or t or g or c
  misc_feature (4805)..(4805): n can be a or t or g or c
  misc_feature (4806)..(4806): n can be a or t or g or c
  misc_feature (4809)..(4809): n can be a or t or g or c
  misc_feature (5164)..(5164): n can be a or t or g or c
  misc_feature (5195)..(5195): n can be a or t or g or c
  misc_feature (5207)..(5207): n can be a or t or g or c
  misc_feature (6668)..(6668): n can be a or t or g or c
  misc_feature (7458)..(7458): n can be a or t or g or c
  misc_feature (7666)..(7666): n can be a or t or g or c
  misc_feature (7687)..(7687): n can be a or t or g or c
  misc_feature (7730)..(7730): n can be a or t or g or c
  misc_feature (8656)..(8656): n can be a or t or g or c
  misc_feature (9403)..(9403): n can be a or t or g or c
  misc_feature (9598)..(9598): n can be a or t or g or c
  misc_feature (10245)..(10245): n can be a or t or g or c
  misc_feature (10817)..(10817): n can be a or t or g or c
  misc_feature (10829)..(10829): n can be a or t or g or c
  misc_feature (10895)..(10895): n can be a or t or g or c
  misc_feature (11001)..(11001): n can be a or t or g or c
  misc_feature (11049)..(11049): n can be a or t or g or c
  misc_feature (11063)..(11063): n can be a or t or g or c
  misc_feature (11067)..(11067): n can be a or t or g or c
  misc_feature (11192)..(11192): n can be a or t or g or c
  misc_feature (11294)..(11294): n can be a or t or g or c
  misc_feature (11336)..(11336): n can be a or t or g or c
  misc_feature (11644)..(11644): n can be a or t or g or c
  misc_feature (12434)..(12434): n can be a or t or g or c
  misc_feature (12531)..(12531): n can be a or t or g or c
  misc_feature (12630)..(12630): n can be a or t or g or c
  misc_feature (12796)..(12796): n can be a or t or g or c
  misc_feature (13123)..(13123): n can be a or t or g or c
  misc_feature (13365)..(13365): n can be a or t or g or c
  misc_feature (13988)..(13988): n can be a or t or g or c
  misc_feature (13991)..(13991): n can be a or t or g or c
  misc_feature (14048)..(14048): n can be a or t or g or c
  misc_feature (14136)..(14136): n can be a or t or g or c
  misc_feature (14346)..(14346): n can be a or t or g or c
  misc_feature (14842)..(14842): n can be a or t or g or c
  misc_feature (14889)..(14889): n can be a or t or g or c
  misc_feature (14901)..(14901): n can be a or t or g or c
  misc_feature (15119)..(15119): n can be a or t or g or c
  misc_feature (15815)..(15815): n can be a or t or g or c
  misc_feature (15879)..(15879): n can be a or t or g or c
  misc_feature (16767)..(16767): n can be a or t or g or c
  misc_feature (16829)..(16829): n can be a or t or g or c
  misc_feature (16832)..(16832): n can be a or t or g or c
  misc_feature (16932)..(16932): n can be a or t or g or c
  misc_feature (16971)..(16971): n can be a or t or g or c
  misc_feature (16982)..(16982): n can be a or t or g or c
  misc_feature (16983)..(16983): n can be a or t or g or c
  misc_feature (16984)..(16984): n can be a or t or g or c
  misc_feature (16986)..(16986): n can be a or t or g or c
  misc_feature (17598)..(17598): n can be a or t or g or c
  misc_feature (17653)..(17653): n can be a or t or g or c
  misc_feature (18106)..(18106): n can be a or t or g or c
  misc_feature (18190)..(18190): n can be a or t or g or c
  misc_feature (18242)..(18242): n can be a or t or g or c
  misc_feature (18266)..(18266): n can be a or t or g or c
  misc_feature (18277)..(18277): n can be a or t or g or c
  misc_feature (18292)..(18292): n can be a or t or g or c
  misc_feature (18319)..(18319): n can be a or t or g or c
  misc_feature (18348)..(18348): n can be a or t or g or c
  misc_feature (18379)..(18379): n can be a or t or g or c
  misc_feature (18995)..(18995): n can be a or t or g or c
  misc_feature (19091)..(19091): n can be a or t or g or c
  misc_feature (19112)..(19112): n can be a or t or g or c
  misc_feature (19809)..(19809): n can be a or t or g or c
  misc_feature (20072)..(20072): n can be a or t or g or c
  misc_feature (20604)..(20604): n can be a or t or g or c
  misc_feature (20752)..(20752): n can be a or t or g or c
  misc_feature (20874)..(20874): n can be a or t or g or c
  misc_feature (21039)..(21039): n can be a or t or g or c
  misc_feature (21552)..(21552): n can be a or t or g or c
  misc_feature (21655)..(21655): n can be a or t or g or c
  misc_feature (21656)..(21656): n can be a or t or g or c
  misc_feature (21657)..(21657): n can be a or t or g or c
  misc_feature (21676)..(21676): n can be a or t or g or c
  misc_feature (21724)..(21724): n can be a or t or g or c
  misc_feature (21731)..(21731): n can be a or t or g or c
  misc_feature (21745)..(21745): n can be a or t or g or c
  misc_feature (21920)..(21920): n can be a or t or g or c
  misc_feature (21956)..(21956): n can be a or t or g or c
  misc_feature (22100)..(22100): n can be a or t or g or c
  misc_feature (22102)..(22102): n can be a or t or g or c
  misc_feature (22160)..(22160): n can be a or t or g or c
  misc_feature (22184)..(22184): n can be a or t or g or c
  misc_feature (22208)..(22208): n can be a or t or g or c
  misc_feature (22267)..(22267): n can be a or t or g or c
  misc_feature (22327)..(22327): n can be a or t or g or c
  misc_feature (22328)..(22328): n can be a or t or g or c
  misc_feature (22329)..(22329): n can be a or t or g or c
  misc_feature (22330)..(22330): n can be a or t or g or c
  misc_feature (22331)..(22331): n can be a or t or g or c
  misc_feature (22348)..(22348): n can be a or t or g or c
  misc_feature (22385)..(22385): n can be a or t or g or c
  misc_feature (22423)..(22423): n can be a or t or g or c
  misc_feature (22683)..(22683): n can be a or t or g or c
  misc_feature (23339)..(23339): n can be a or t or g or c
  misc_feature (23423)..(23423): n can be a or t or g or c
  misc_feature (23463)..(23463): n can be a or t or g or c
  misc_feature (23469)..(23469): n can be a or t or g or c
  misc_feature (23499)..(23499): n can be a or t or g or c
  misc_feature (24198)..(24198): n can be a or t or g or c
  misc_feature (24522)..(24522): n can be a or t or g or c
  misc_feature (25144)..(25144): n can be a or t or g or c
  misc_feature (25458)..(25458): n can be a or t or g or c
  misc_feature (25470)..(25470): n can be a or t or g or c
  misc_feature (25842)..(25842): n can be a or t or g or c
  misc_feature (26054)..(26054): n can be a or t or g or c
  misc_feature (26088)..(26088): n can be a or t or g or c
  misc_feature (26519)..(26519): n can be a or t or g or c
  misc_feature (26954)..(26954): n can be a or t or g or c
  misc_feature (26975)..(26975): n can be a or t or g or c
  misc_feature (27016)..(27016): n can be a or t or g or c
  misc_feature (27079)..(27079): n can be a or t or g or c
  misc_feature (27092)..(27092): n can be a or t or g or c
  misc_feature (27093)..(27093): n can be a or t or g or c
  misc_feature (27672)..(27672): n can be a or t or g or c
  misc_feature (27788)..(27788): n can be a or t or g or c
  misc_feature (28202)..(28202): n can be a or t or g or c
  misc_feature (29025)..(29025): n can be a or t or g or c
  misc_feature (29232)..(29232): n can be a or t or g or c
  misc_feature (29325)..(29325): n can be a or t or g or c
  misc_feature (29352)..(29352): n can be a or t or g or c
  misc_feature (29355)..(29355): n can be a or t or g or c
  misc_feature (29617)..(29617): n can be a or t or g or c
  misc_feature (29618)..(29618): n can be a or t or g or c
  misc_feature (29619)..(29619): n can be a or t or g or c
  misc_feature (29623)..(29623): n can be a or t or g or c
  misc_feature (29628)..(29628): n can be a or t or g or c
  misc_feature (29635)..(29635): n can be a or t or g or c
  misc_feature (29680)..(29680): n can be a or t or g or c
  misc_feature (29693)..(29693): n can be a or t or g or c
  misc_feature (29796)..(29796): n can be a or t or g or c
  misc_feature (29816)..(29816): n can be a or t or g or c
  misc_feature (29823)..(29823): n can be a or t or g or c
  misc_feature (29844)..(29844): n can be a or t or g or c
  misc_feature (29923)..(29923): n can be a or t or g or c
  misc_feature (29928)..(29928): n can be a or t or g or c
  misc_feature (29964)..(29964): n can be a or t or g or c
  misc_feature (29984)..(29984): n can be a or t or g or c
  misc_feature (30017)..(30017): n can be a or t or g or c
  misc_feature (30024)..(30024): n can be a or t or g or c
  misc_feature (30034)..(30034): n can be a or t or g or c
  misc_feature (30037)..(30037): n can be a or t or g or c
  misc_feature (30793)..(30793): n can be a or t or g or c
  misc_feature (30976)..(30976): n can be a or t or g or c
  misc_feature (31273)..(31273): n can be a or t or g or c
  misc_feature (32695)..(32695): n can be a or t or g or c
  misc_feature (32850)..(32850): n can be a or t or g or c
  misc_feature (32854)..(32854): n can be a or t or g or c
  misc_feature (33053)..(33053): n can be a or t or g or c
  misc_feature (33070)..(33070): n can be a or t or g or c
  misc_feature (33549)..(33549): n can be a or t or g or c
  misc_feature (33571)..(33571): n can be a or t or g or c
  misc_feature (33894)..(33894): n can be a or t or g or c
  misc_feature (34106)..(34106): n can be a or t or g or c
  misc_feature (34395)..(34395): n can be a or t or g or c
  misc_feature (34621)..(34621): n can be a or t or g or c
  misc_feature (34640)..(34640): n can be a or t or g or c
  misc_feature (34641)..(34641): n can be a or t or g or c
  misc_feature (34645)..(34645): n can be a or t or g or c
  misc_feature (34646)..(34646): n can be a or t or g or c
  misc_feature (34649)..(34649): n can be a or t or g or c
  misc_feature (34656)..(34656): n can be a or t or g or c
  misc_feature (34707)..(34707): n can be a or t or g or c
  misc_feature (34737)..(34737): n can be a or t or g or c
  misc_feature (34746)..(34746): n can be a or t or g or c
  misc_feature (35432)..(35432): n can be a or t or g or c
  misc_feature (35625)..(35625): n can be a or t or g or c
  misc_feature (35802)..(35802): n can be a or t or g or c
  misc_feature (36086)..(36086): n can be a or t or g or c
  misc_feature (36269)..(36269): n can be a or t or g or c
  misc_feature (36736)..(36736): n can be a or t or g or c
  misc_feature (36783)..(36783): n can be a or t or g or c
  misc_feature (37338)..(37338): n can be a or t or g or c
  misc_feature (37456)..(37456): n can be a or t or g or c
  misc_feature (37457)..(37457): n can be a or t or g or c
  misc_feature (37914)..(37914): n can be a or t or g or c
  misc_feature (38619)..(38619): n can be a or t or g or c
  misc_feature (38664)..(38664): n can be a or t or g or c
  misc_feature (38787)..(38787): n can be a or t or g or c
  misc_feature (38789)..(38789): n can be a or t or g or c
  misc_feature (38791)..(38791): n can be a or t or g or c
  misc_feature (38808)..(38808): n can be a or t or g or c
  misc_feature (38811)..(38811): n can be a or t or g or c
  misc_feature (38813)..(38813): n can be a or t or g or c
  misc_feature (38815)..(38815): n can be a or t or g or c
  misc_feature (38817)..(38817): n can be a or t or g or c
  misc_feature (38819)..(38819): n can be a or t or g or c
  misc_feature (38820)..(38820): n can be a or t or g or c
  misc_feature (38821)..(38821): n can be a or t or g or c
  misc_feature (38822)..(38822): n can be a or t or g or c
  misc_feature (38823)..(38823): n can be a or t or g or c
  misc_feature (38825)..(38825): n can be a or t or g or c
  misc_feature (38826)..(38826): n can be a or t or g or c
  misc_feature (38827)..(38827): n can be a or t or g or c
  misc_feature (38832)..(38832): n can be a or t or g or c
  misc_feature (38834)..(38834): n can be a or t or g or c
  misc_feature (38835)..(38835): n can be a or t or g or c
  misc_feature (38836)..(38836): n can be a or t or g or c
  misc_feature (38838)..(38838): n can be a or t or g or c
  misc_feature (38840)..(38840): n can be a or t or g or c
  misc_feature (38842)..(38842): n can be a or t or g or c
  misc_feature (38850)..(38850): n can be a or t or g or c
  misc_feature (38852)..(38852): n can be a or t or g or c
  misc_feature (38854)..(38854): n can be a or t or g or c
  misc_feature (38856)..(38856): n can be a or t or g or c
  misc_feature (38858)..(38858): n can be a or t or g or c
  misc_feature (38862)..(38862): n can be a or t or g or c
  misc_feature (38864)..(38864): n can be a or t or g or c
  misc_feature (38873)..(38873): n can be a or t or g or c
  misc_feature (38875)..(38875): n can be a or t or g or c
  misc_feature (38877)..(38877): n can be a or t or g or c
  misc_feature (39427)..(39427): n can be a or t or g or c
  misc_feature (39428)..(39428): n can be a or t or g or c
  misc_feature (39435)..(39435): n can be a or t or g or c
  misc_feature (39436)..(39436): n can be a or t or g or c
  misc_feature (39439)..(39439): n can be a or t or g or c
  misc_feature (39440)..(39440): n can be a or t or g or c
  misc_feature (39441)..(39441): n can be a or t or g or c
  misc_feature (39443)..(39443): n can be a or t or g or c
  misc_feature (39445)..(39445): n can be a or t or g or c
  misc_feature (39446)..(39446): n can be a or t or g or c
  misc_feature (39447)..(39447): n can be a or t or g or c
  misc_feature (39448)..(39448): n can be a or t or g or c
  misc_feature (39626)..(39626): n can be a or t or g or c
  misc_feature (39649)..(39649): n can be a or t or g or c
  misc_feature (39671)..(39671): n can be a or t or g or c
  misc_feature (39961)..(39961): n can be a or t or g or c
  misc_feature (40088)..(40088): n can be a or t or g or c
  misc_feature (40602)..(40602): n can be a or t or g or c
  misc_feature (40653)..(40653): n can be a or t or g or c
  misc_feature (41601)..(41601): n can be a or t or g or c
  misc_feature (41796)..(41796): n can be a or t or g or c
  misc_feature (41850)..(41850): n can be a or t or g or c
  misc_feature (41863)..(41863): n can be a or t or g or c
  misc_feature (41887)..(41887): n can be a or t or g or c
  misc_feature (41984)..(41984): n can be a or t or g or c
  misc_feature (42114)..(42114): n can be a or t or g or c
  misc_feature (42436)..(42436): n can be a or t or g or c
  misc_feature (42630)..(42630): n can be a or t or g or c
  misc_feature (42631)..(42631): n can be a or t or g or c
  misc_feature (42632)..(42632): n can be a or t or g or c
  misc_feature (42652)..(42652): n can be a or t or g or c
  misc_feature (42715)..(42715): n can be a or t or g or c
  misc_feature (42850)..(42850): n can be a or t or g or c
  misc_feature (42902)..(42902): n can be a or t or g or c
  misc_feature (42946)..(42946): n can be a or t or g or c
  misc_feature (43373)..(43373): n can be a or t or g or c
  misc_feature (43408)..(43408): n can be a or t or g or c
  misc_feature (43409)..(43409): n can be a or t or g or c
  misc_feature (43512)..(43512): n can be a or t or g or c
  misc_feature (43657)..(43657): n can be a or t or g or c
  misc_feature (43765)..(43765): n can be a or t or g or c
  misc_feature (43777)..(43777): n can be a or t or g or c
  misc_feature (43825)..(43825): n can be a or t or g or c
  misc_feature (43905)..(43905): n can be a or t or g or c
  misc_feature (43934)..(43934): n can be a or t or g or c
  misc_feature (44380)..(44380): n can be a or t or g or c
  misc_feature (44422)..(44422): n can be a or t or g or c
  misc_feature (44559)..(44559): n can be a or t or g or c
  misc_feature (44790)..(44790): n can be a or t or g or c
  misc_feature (45124)..(45124): n can be a or t or g or c
  misc_feature (45125)..(45125): n can be a or t or g or c
  misc_feature (45155)..(45155): n can be a or t or g or c
  misc_feature (45157)..(45157): n can be a or t or g or c
  misc_feature (45158)..(45158): n can be a or t or g or c
  misc_feature (45172)..(45172): n can be a or t or g or c
  misc_feature (45468)..(45468): n can be a or t or g or c
  misc_feature (45714)..(45714): n can be a or t or g or c
  misc_feature (45987)..(45987): n can be a or t or g or c
  misc_feature (46036)..(46036): n can be a or t or g or c
  misc_feature (46042)..(46042): n can be a or t or g or c
  misc_feature (46056)..(46056): n can be a or t or g or c
  misc_feature (46090)..(46090): n can be a or t or g or c
  misc_feature (46094)..(46094): n can be a or t or g or c
  misc_feature (46110)..(46110): n can be a or t or g or c
  misc_feature (46112)..(46112): n can be a or t or g or c
  misc_feature (46114)..(46114): n can be a or t or g or c
  misc_feature (46121)..(46121): n can be a or t or g or c
  misc_feature (46134)..(46134): n can be a or t or g or c
  misc_feature (46144)..(46144): n can be a or t or g or c
  misc_feature (46146)..(46146): n can be a or t or g or c
  misc_feature (46149)..(46149): n can be a or t or g or c
  misc_feature (46176)..(46176): n can be a or t or g or c
  misc_feature (46177)..(46177): n can be a or t or g or c
  misc_feature (46200)..(46200): n can be a or t or g or c
  misc_feature (46223)..(46223): n can be a or t or g or c
  misc_feature (46447)..(46447): n can be a or t or g or c
  misc_feature (46713)..(46713): n can be a or t or g or c
  misc_feature (46886)..(46886): n can be a or t or g or c
  misc_feature (46906)..(46906): n can be a or t or g or c
  misc_feature (46987)..(46987): n can be a or t or g or c
  misc_feature (47006)..(47006): n can be a or t or g or c
  misc_feature (47406)..(47406): n can be a or t or g or c
  misc_feature (47442)..(47442): n can be a or t or g or c
  misc_feature (47729)..(47729): n can be a or t or g or c
  misc_feature (47851)..(47851): n can be a or t or g or c
  misc_feature (48512)..(48512): n can be a or t or g or c
  misc_feature (48051)..(48051): n can be a or t or g or c
  misc_feature (48512)..(48512): n can be a or t or g or c
  misc_feature (50116)..(50116): n can be a or t or g or c
  misc_feature (50789)..(50789): n can be a or t or g or c
  misc_feature (50790)..(50790): n can be a or t or g or c
  misc_feature (50791)..(50791): n can be a or t or g or c
  misc_feature (50793)..(50793): n can be a or t or g or c
  misc_feature (50794)..(50794): n can be a or t or g or c
  misc_feature (50795)..(50795): n can be a or t or g or c
  misc_feature (50796)..(50796): n can be a or t or g or c
  misc_feature (50797)..(50797): n can be a or t or g or c
  misc_feature (50799)..(50799): n can be a or t or g or c
  misc_feature (50801)..(50801): n can be a or t or g or c
  misc_feature (50802)..(50802): n can be a or t or g or c
  misc_feature (50803)..(50803): n can be a or t or g or c
  misc_feature (50805)..(50805): n can be a or t or g or c
  misc_feature (50806)..(50806): n can be a or t or g or c
  misc_feature (50807)..(50807): n can be a or t or g or c
  misc_feature (50808)..(50808): n can be a or t or g or c
  misc_feature (50809)..(50809): n can be a or t or g or c
  misc_feature (50811)..(50811): n can be a or t or g or c
  misc_feature (50812)..(50812): n can be a or t or g or c
  misc_feature (50813)..(50813): n can be a or t or g or c
  misc_feature (50814)..(50814): n can be a or t or g or c
  misc_feature (50816)..(50816): n can be a or t or g or c
  misc_feature (50817)..(50817): n can be a or t or g or c
  misc_feature (50819)..(50819): n can be a or t or g or c
  misc_feature (50820)..(50820): n can be a or t or g or c
  misc_feature (50822)..(50822): n can be a or t or g or c
  misc_feature (50824)..(50824): n can be a or t or g or c
  misc_feature (50937)..(50937): n can be a or t or g or c
  misc_feature (51301)..(51301): n can be a or t or g or c
  misc_feature (51327)..(51327): n can be a or t or g or c
  misc_feature (51896)..(51896): n can be a or t or g or c
  misc_feature (52444)..(52444): n can be a or t or g or c
  misc_feature (52666)..(52666): n can be a or t or g or c
  misc_feature (53747)..(53747): n can be a or t or g or c
  misc_feature (54085)..(54085): n can be a or t or g or c
  misc_feature (54153)..(54153): n can be a or t or g or c
  misc_feature (54251)..(54251): n can be a or t or g or c
  misc_feature (54520)..(54520): n can be a or t or g or c
catgagcagaaggcagatgacagctgccataatggaaaatagtatttcctcataaatttccttgatctaggccagttctcaggctcagagcctattgatttaaggaaaggccaagcctttttatagaagagccctccaatgctgccaaaggtatacaagataattatttccccaatctttccacatgagagatctgtggccatttatcatagtaactctggtcagggggaagaaaaagaaccagactttcaaaggctattgggtacagagtatgagctggcatcagtgctggggggaaacactatcatggtccccccgtgacagaggctcatgaaagccaggtaaatggantactcttgcaaacagatccagaagtctgaagatccacactgtactaatttccttatccctaagtaaataactaggacagatgttntagcaactggcagtacactaacatttactccctattttattaagagccattaaggtaggaagggccaaatgaaagcccctgacacttaccctcacctttcttactgatttattaaatgagaagtaaaaccacatcctgggtggtattgcagagattaatttcaacatcaaatattttcttctcaatatccttcagtcaggaggatgaaaaactgtttgcttttatgtaggaataatagcagtacatattcactctcttgcctctgggctatgttagttctcctctctgcacaacacagtatctagcgactttgattgtctcaatattccattgaacatcatggtagtccactgtattgatgacctaatgctaatttgacctagtaagcaagaagaagcaagaactctagatgtcctagtaaagacacatgtatgccaaaggatagcagataaaaatatgaagattcaggacattcatattgttcaacttttttcttacntttttgttctagaataagttaaatggatgtaaaagtagacaaactagtaaaacgaatcccttgaaccactattcacagcccatattatttcatctatatccccaccaacttcttctatcccatattattttgaaacaaatgggtaaagttatcagggatcagtagtcatgccaggttatcccctgtaaagtaaagacaagttattgagctctgtatcctccaacactaaggcatagtgcttggtggagtattcctctgacccatctatttcgtgcccctgaaggctttcagtttcgggtgaaacccagagcaggagagagctgtacagtgtgtccaactagagtacaagcttctctaccatttggttcatattatccagcagatacaatggggctagaggcatacatgatgaataaggctacagggcagagtctctgacatgccctaataaaagagtttcagcacagatctctaaagttctggagcaaggccatgcagcagagagatattcgacttttgaaaagcagctgtttggccccaataaaaaccaagagcctagcaatgggacatcaagggtctatgcaaccagagatacccattgtaagccaggtattgtcagagtctaccaagtcgtaagattaggcaggtttaagccaggcatggtggctcacacctgtaatcccggcactttgggaggaagctgagggcagagaattacttgagcccaggaggcaacatggcaaaaccccatctctaaaaaataaagaaaaaaaaattagctgggtgtggnggtgtttgcctgtagtcccagcaacctgggaggctgaagtagaaggattgcttgagcctagggaggttgaggctgcagtgagctgtgattgtgccactgcactccagcctgggtgacagactgagaatctctctcaaaaaaaaaaaaangcagacttatcagcaatccatcatataataatagaacaggaatggtaaattcaggatcaggactaagcaggtcaatagggtgcatgtaagtcaacaaaaggtggctcagactcctatgacacctacctcagctatcggctataccaacccttctgtnttagctcagacctacagccttagtaggtagttctctgtaaccaactgatggaggggaaaatagcccttactcacagatggattagctttagacattgatactagttaaaaaagaaaaaaanaaaaagagattgttcctgtaacatagctccatttagggggtagccctaaacaactttgaagggaaattcccccagtgggctaagctgtaagcagtatacttgttcattcattttgtatggaggacaaaacaattccagatagagntttacatacacttgtgagcaatggcaaatagtcgagtggtacctgttcagccctcacttatgtgcaatctggaagtgcaagcagttgaccaatggagacagaactaggaaacaaatacctcctcattctatatggctctttagaagcccccagtgggattaaacctcagtaacccacagcaatggggattgagacaaggctgaggtgaggaaaaagagcaaggcacttgtctctggtgcaaatttaaggaggcaccaagaaattcagtaaccaagattaattatattttaatgcaatattttttaaaatagaaattaatgcaaatacccatgatgaacgcaatatcaaaattttcaataaagacaggctcagtattgctaattttccatttattcttaggctccactagggctccacacaacactgttactgatcttggtcatcgccaanaaccatctcaatttcacagtctatgttggctttacctccctctctgttcactctcctccctagtccctcacttctgttttatggtatcattactcagaataaacgaccttatatacgttcttgtctccaactttgctctattatatggggtggagtagggagatgaaacccaagctaagtaagataatgaccataatatatcattcgaagaaggatacttgtgaaagtaaagggaggctctattaataatttcactggattaataggcataaatcagagctgtcctagcacatcaggatgtatagtcttattacaggtaggaaactgaggttccaagggggtgggaagtcctcctaagaggaggggtcagactggaagctacacaaaaagaggcagaaatgtccctccgcctcttaatcggagcctggggaaacaaacacactcgtgcaaaatctccaaagaggtgagctcttcaccctgtcaattttatctgcctcgtgaaccccagggctcaggctccaggcccgggctagctcacacaacagcagaagggccttaggtctgggcactgagccaaaaaaagggaggctgtggtccctgctttggttaccctgccgggtctgagcgccaaatcggatctcctacttctctctccctccctccctctgttcctggactgggggcaggcccttctgctaacagaaagccttccagtcggagaaagctctgncagcctgccgcgataaagggccctgggggcgtgaggagagcaaggggctcaaaatcanaacccagcgtgacgacagtgcctccatgactgtgctcccgcaatgcgcaggctccatgtcctggcgcccgcccgcccatggacccggcgggggcttccaggctgggctcagccattacgccggcgtgcgggggaggaaactcgcctcccgggcactcggttgtctcctgcccccgcccctccctccgatccgggcccatctctgacgtagtgtgaccttgctcatcccttccaggctgtgggcctgttttccctgtgcaagatgagggtcctggctgtcctgaggacgctgtccgggcgccgccaggggtgaccgaattcagctctgctaggactgttgggaaatgagctccctgtcggcgtgtgccagccgcctgcgcgaggcgccacaggagagggcgcgctcctgtcgctctgccgcccccagaagtttcccgggaaccgactccactgactcgcccctccgcgccccacgcgtggcagcctaagctcagcctccanattggaggagaccgcggagggaaccctgctggggtctgggcccggggcnacncggcccgagcagatcgagggccgacccctccgagaactcgctctctggcctcggctcctcccttgcgcccgccctcccacgtggggcccaggtctgggaatcagcgctcgggggtggctggggacaaccgagaacgagcttcttccccggcacgcgggcggaatggctgagcccagcctggaagcccccgtcaggtccttgggggcgggcgggcgcgcgaagcacagggcgganacagccgggagcccagcctcccgggctgggccgccctccccttccccgcgccnggccggggatgggggtgtgntcccaagtgtacagtggcatcaagctcagcgcganctcccgggaacgctccaacgccttcagcctgtttcccaggaacggtccccngnttcgcgccccaatttctaacagcctgcctgtcccccgggaacgttctaacatccttggggagcgccccagctacaagacactgtcctgagaacgctgtcatcacccgtagttgcaagtttcggagcggcagtgggaagcatgcgggactacgacgaggtgatcgccttcctgggcgagtgggggcccttccagcgcctcatcttcttcctgctcagcgccancatcatccccaatggcttcaatggtatgtcagtcgtgttcctggcggggaccccggagcaccgctgtcgagtgccggacgccgcgaacctgagcnnnnncnngcngaacaacagtgtcccgctgcggctgcgggacggccgcgaggtgccccacagctgcagccgctaccggctcgccaccatcgccaacttctcggcgctcgggctggagccggggcgcgacgtggacctggggcagctggagcaggagagctgcctggatggctgggagttcagccaggacgtctacctgtccaccgtcgtgaccgaggtgggtgccaggccgagaccgttgacccgggagtgcctgaccctccctctgcgtcagccccccttgagactccctgcgcagtgcccgggtcagcgctcccctccccctcaaacctgctgttcatacttccagcagcgggtgtgcaccccnagaaagaataggactcacgcgaggcgcattnctgggtcgtttntgtccaataaggaggtgatggagaaaggagtttgtcaccaactgtcttttcatttccgattttcacaacaaatgaagtttcctaagaacctagatgttgcgttgggggaagcgcagcccaggctacccagacgcactgcctgagtcaccttcctgccaggcatgggaggaggggtgtgagggaccttgttactgggcagggcggggccaaatcttgtctgttccctggccacagacttctggaggcatcaggaggtgtactgccgtaccatatacttgtgaggtttggggtcctttcctgaggccccagtttctctaagacagcagatggcctcctgaggctcattgggtgcaaggaagcacagaaggcgaaaccccagctggaagccctggccctcagggacctctctgagcacagggtgagttggcaggcaggggctgtagcccctcagcaccctgcgtggttagtggcaggttttctaagttccaggaatgaggatgcacacacacttctcccgcttttgctgcttcacatctggtaaggacctgggtcaggatacacctttcttctgaggagagcatcctcatggagctgttgcctgctattggtcaggcaggtaggatttccctgctgctagtccctcctctgcctcatgctcctcccaagaaaaacctgagtggcctctgctagtcccttatccctttccctcagcacggtatagcggtcatcagacattgtgtctggccttgtgctgagcactaaggtgtggacatctggtgtgtggagcctaccctcagatgcccatggggcacactggctgtgttactgagaaggctaacattaaccctggttagcaatagggttctggaacccaaattcaagtcctgccttgcttgctatgtgacctgggataaataaatcaccctctctgtgggacaataggtggaggaaatactccaagaaggggtgatatgattttagctgattctgaaaatgtgagcaagacctgtctaggcaagagtatgggaaattcaggtaatggaattctcatatatcggagtggctggaactcaagggatgacagatatgacaaatgagactagaaatggccacaagggctagttcatagtggccttctatgtatactgaggagctgactttattcggaggacagtgagaaaatagtaagcattctaaggccaggttcgtgcttttggaaagagctcactctagctgctatgataaacaagaagaccagtgaggaagttttgaaactctcctctgcaagagaatggtggccagccaggcgtggtggttgtcgaatctgtggcacctgtgggaagtgggctcagcccagggactgcctttggatcccccagcattggcaccatacctaccctgggccctaaggtggccatgcttctctggatttgcttccctagcaggggctctagtgcttgnccactccctgcccacagcatggcctgggagcagctgagactgggggccagctcatcccgcgtcgattcctggaagtgttatcagtgcctgttatggaggctggacccatgagtggcagccttccctggcagctgggctgacctgtctgcttttccattgctcgctggttttgttcactgtagggcgtgaggggtgagtagctgctggcctccaagtccatagctactcatgttgtacgctgttcacagggacctctaaggatgtacatcatcacacattcacacacatgcaccaggatttgctttttttggcagcttttcccttcctggcttcctttttgagtggtggaagtaaaataaaaagcaactaggggctgggcacagtggctcacgcctgtaatcccagtactttgagaggctgaggcgggcagattacttgaggtcaggagtttgagaccagcctggccaacatggtgaaaccccgtctctactaaaaatacaaaaattagctgggagtggtggtgcacccctgtagtcccagctactcgggaggctaaggcaggagaatcacttcaacctgggaggcggaggttgcagtgagctgagatcacaccacagcactccagcctgggtgacagagccagactgtgtctcaaaacaaacaaacaaacaacaacaacaacaaaaaactgggctctggtggttgggaggaggagggaaggaagcaagtaccagggtaagcaggatggatggatggctctcccccagaggggcggcagcacacagagttntggagtcagactcagtagagggccagttttgactccactgccaaccacctggctgactccaggcaggttacatcactgatgaaagcctcagtttccttgtctataaattgggggtacaagcgatgaaaagaggctcaacatcactaatcactagggaaatgcaaatgaaaatcataaggaggtaccactttgtacccttaaaaatagntactacaaaaagaaaaaaaanacacccagaaatccactttgggaggccgaggcaggaggattgntagggggcaggagttcaagacagcactgggcaacatagtaagaccccatctctacaaaaaataaataaaaaattagccaggcatgagggcatgtgactgtagttccagcttctcaggaggctggggcaggaggatcacttgagcccaagagtttgaggttgcagtgagctatgaccatatctctgcactccagcctgggtgacagggcaagaccccacctctaaaaaagtgttgttgttgtttttaaacacagaaaataacaagtgttagtgaggatgtagagaaattgaaacccttgttcattcctagtgggagtgtaaaatggtgcagccactatggaaaataatgtggtggtccctaaaaaaattaaaaatagaatcaccatatgatccagcaatactaattctggatatatgtctaaaataattgaaacagggtttcaaagagatatttgtacaccatgtctatggtagcattattcacaatagtcaaaaggtgaaagcaacccaagtgtccttccacagacaaatggataaacataatgtggtatatgcgtacaatggacgattattcagctttaaaaaggaaggacatgctacaacgtggataaaccttgaggagactacgcttagtgaaataagccagtcacaagacaaattattttctgatttcactacaggagtagtcacactcacagaagcagaaagtaaatggtggttgccagggactggaagaaggaaggagtagggagtcgttgtttcatgggtatagagtttcagttttgtaagatgaaagtagtgttggagattgattgcacaacagtgtgaacgcttctgaactgtacacttaaaaatggctaagatggtaaactgtatattatgtgtactttatctcagttttcaaaatggggataataagagaagccnccttatctgagttgttgggaggactcagtgagataatgccccatgccccatccggtggtggccagcacagagtgtggcaccagcaaagagtgtggcaccagcaaatgggcttcctaggtggtggtgaggagtggtaaaggcagaagctcagcaccttctttgccttctccactggctggggacagtgggcatggtgggagctgttctaaagtccaggttgtggcctgtgtaataggtgatgggacttgctggaggctgagcccctctccccacagtggcagttccaagcctcagagaatagaacacaagtccaaacccatactgacttggccagtgccagtgtacagggccaagcccagggcccctgagaaggttctctgcatcccagttggcacaacacaggtagtgggactgtcctcagattgtgtctttgccttatactgccgtgccaggatctcccaatgggcatatctttggggacagacaagtctgcctgaactgccccccaacacctcttcacaggaggacagggttagccaggaaccaaggatggcatgctgtgctgtagaaatgcctttcatagtatgccaggaggtagcaataaggtgtctgagttggggtttgcgtgagtgcattcgtctgtgtgtgtacatgtgtataaggtagtttccttcttcaggggttgattgtgaggtccaagtttttcaacctgtgcagttactaagagtcaaattatttttcgttttnttttattttattttattttattttatttttttgagatggagtttctttcttcgttcttgttgcccagggtggaggagtgcaatggtgccatctcggctcactgcaacctctgcctccttggttcaagcgattctcctgcctcagcctcccgagtagctgggattacaggggcctgctaccatgcccggctagttntttgtttgtttgtttgtttgttttttgtatttttagtagagacagggtttcaccatgttggccaggctggtctcgaactcctgacctaaggtgatccacctgcctcggcctcccaaagtgttgggattacaggcgtgagccaccgcgcctggcgattattttttaactttattttgaaataacttttgatttagagagaagtttgaaaatagtacagagatttcacatatacccttcatctagctttctctgatgttaacaatttaaatacgcgttatgcatttatccaaaccaggaaattaacattggttacaatagtaataactgaactacagactttgtttggatttcaccagtttttctactactgccctttttctatcgtaggatcccatccaaaatcccatgttgtatttagttgtcatgtctcagtctccaatctgtgattgttccttagtctttccttatctttcatgatcttgacatttttatgagtattttgatgaatgctcctcagtttgggttggcatgatttttctcacaattagattgcgttatgcactttttggtaatgataccttagaagtgatgcattctctgggcatcataccagtgggtacatgatgctgatgtgtcttattactggngatgttaaccttgatcatttgtcaagttggtgtctgtggaatttctctgctgtaaaggtactattttttcctctgtcactaataaatatcttaagggaggttcttcgagattatacaaaatcctgtgtctcctcaaatttttattcactgattttagcattcatcactaggtcttgactgaaacaattattactgcagtttgcctagtgatgattttgcattttctgattccttcaacatttattaattggaattcttctgagaagaagagcttcccctccacatctatttattaatgtatgcaattgttaacttatatcagtacaggttcatacatacttattttatttcatcagttataaactaatgctatccatctttattttgttgctccagttattctagctcaaaccaagttatttttcactttaaggatagagaatgtagatatccagggtaagggcctttatctgtctctgcagtcagggctagaggagtctgtgtttacccctgcgggaggagtgccagtcacagctcgttgctaagttttccatcaggcagcagttgagcangaacgcaagcanggactggtgggacctgggttgggactgaagcttctgttggggaaggaagtgccccaagcaacccanagtgtctgatgctggggtcctcatccctgagacctactgcaggggtcagatctcagtctccatgggcatggcccttcatatatagccctcagggccaagacctcanagagaagaggccctgcgtgtgaggaaacccggcagcctcctgccctgnggctaaaggagcangaanggcaacaaatgctctgctctggagagggcctaagttcatctgcagggccacagggcaagcagcagaggcaatcctggctcgtggccaggggctaagcatgggctggcctttgcctcaagcatgtnctcttctcccatggagcatcattagggtcttgaccttcccatcagtccatgggctgggaaaactgaggcagtcacctctaagctggataccacttctgccangtcttgtgctccagaggccctgggggaaccttgtgtgttaangaccagccttctttgcaggccaccagtgatctaagacagggctgctgccacaccgtagatcctcctgtcctcttctgcagctgggctgcctcctctaggaaccctagtggagctgctggtagcccactggtagtggagttgatggagcctccagcaaagggcctggacctctggtggcactagccttgtgctttcctgggaactggggtcagggcctgaacagcagtaggacttactgattccccacccataacccagaggcgagccaggttatgtggcgaaggataaggcctcttcccctgctaacngggaacttctgggaggcactgagagcagcaagcaagggaataatgaaacagactaaccctcacatgcggctgccactatgtgggacttcacatagcggctctcaaggatgcacctcccctggggtctgggtaggccccgacaaaaggggaaaaccttccgtgataggcaggtttgtggagtttctatcaggcctgcaggctgccctgggacattgctcacgggtgtggagactgtcaagatagcctgaatattaaccaagttgatcatccaagtccatactcagacctgccctctggcagattccaaaggggataaggtgtgggggcctctggctccagccagcccggcgagacttctatttccagtcaatgcttcagaaagtagatcttgccagagagcacatagaggagactcaggggcttccccttgctctggcctgaatgtgcttccctgtttgtttatcagctgcctcaccccaaactgtttgcctctctgctgtgttcctctgacaacagccaccaggatctcagaatatgcttttgttcagaggctgcttcacctgcacctcctgccaagtttctggataaacagactttgtccatccctcttggtgggcagcagaactctcagacagagtccaaggtggcattttgacaaaaacaaggctttgacaggactggtcagggggagctgcaggtaccttgggcctctagaaggactgtaattcctcccttatggcaggctgcattcaatttaggtgtaagactaatacaggtgtgccttgttttntaaagctgtgttgtggttttatgcatgtctctggagtccaaaagaaaggcaaggatccccatttggaatgaagaaggtgtggagagaatggacgttntggggaaaggctggaatgacccacatgctctagcaagaagcacagacagtccaaggctggtggctcctgctcagcatttgcaagactggagggcaggtncaggcggcaacttccagagcttcccctcagtgcttggtgactggcacagacacgatgccatttgctcatgttcccctaatacagactggtagagctgtggtggatccattgtcaggatgcaaggggcacagccaggtccctgagtgacccttctccttcccacagnccctccactcccatccctatcttcccactcacactcactggttcttctgctttggtttagcctccaatgatcacagaactcatcagtgaggcttttccatctgtggtagcttttctaatataaagaaagaaagatactgagaacattttaaggcttagcagccttctggcttagttgtttgtaaatgaaaatatgagtagaagaattaaacagccaaaaagataacccacaaaatgggagaaaatattgacaaatcatatatctgataaaggatttgtatctagaatataaaaataactctgataactcagcaataaaaagacaacntgatttaaaaatggataaaatcatttgcccattttggagaatagatgttttgccaaagaaatacaaatcaattagcacatgaaaagattctgagacttcgaagttagcagtgtgaggagcgccagcgccatagaccagctcctcagtgaaacagccataactggtgaaaatacacacacacacacacacatttagagtctctggaagttgtcctaagggcatatatttattcaagaaagtcnactataccttggcaagaacagtgaagtctatgatatttgagtcataactccttcagccctccccttccccttcctcccagcacaagaaaaatggaagttccactctaggtgggtatggccaagaaaatactgttctctctcccatcagcttccaaggaagggctacagcagcccaccaagagggacaaccaatgcccatcccccacccatccctgtattctcttagctccaaaccacagaaggtaaaatcctcgtgagtgcaaccaggggatcataggttcccatcctccacccaccgcctattcatatagtagagactctactccaggtgccacaagttgagaatattagagccttgattgcccttatcccagctcattcatacggcagagattccacaccaagctcccacttccactcagtgtctgactcataaagtaggggtgtcattctgagataaatgggccgctttccctgcctacctctctacaacaggagctcagatattttgcccagcggggagagataatccaaaagaacagagttctaaagctgtccccacaggaacgaactttatttgaaatagagtgtgtgaaaggacagcctaagattgctctcaagancantggagaatttagtggtaagcaactaaaaggattctggtaactccattagagcaacanatgaacccataatgcagctagtttaccaggaaaaaacagaaaacaaacaaacaaacaaaacaagaaagagacagctaagagcagcccntctggagtcaaaacaaaccttatagatttgcctcaaaaactaccactgcaaagaggcttgacgtcaactggattagactgtgggacaatatatgcccccagggcattattgaaatgaatgaggcaactggctagcaattagtgaggctaacagctgggtgtgataccagcagacacagacagcttaatagaaagatcagactggcgtggnggctcatgcctgtaatcctagcactttgggaggccaaggcgggtggatcacctgaggccgggagtttgagaccagcctggctaatgtggggaaaccccatctctactaaaaatacaaaaattagctgggcatggtggcaggtccctgtaatcccagctactcaggaggccgaggcaggagaattgctggaacccaggaggtggaggttgcagtgagccgagatcatgccactgtactctagcttgggcaacagagtgagacttcatctcggaaaagaagaagaagaaagatcagggaaatgggggtggaagaatgtgacagtacctaatttctacaatatattatctaaaatgtccagtttttaaccagaaattacgagatatgcagagaaacagaaaaatatgacctatacatggggggaaaacgtgggcaacagtaactgcctgagagggtccacatgtcagattttaacagactttaaggcataaggcagccnttataaatatgttcaaagaatgaaaggaagtcatgcttttaaaagtntgatgacaatantgcatcaaatagagaatagcaataaagagagaaaaattacatttatatttttaaaagctgagccagacatgatggcatgtgcctatagtcccagttactcaggaggctgaggtaggaggattgcttaagcccaggagttcaaccccagcctgggcaacagaaagagaccttgtctcttaaacaaacaaaacaactgaatagaacttctggagtaaaantatacaataattgaaatttaaaagtaactcaagggccttaacagaagatttgaacaggcaaaagaaagaatcagcaaacttgaagatagaggattggaaattatgcaatccaaagaacagagaggaaaaagggaataaagaaaatgtatagagtctcagagaaatggggaacaccattaaacatagcaatatatgcataatagaattactaggagaggagagagagagaaaagggcagaaaaaatgctcaaagaaataatgactgaaaacttcccaaatttggtggaaaacattaatctacacctttcagaagctcaacaaactccaaatagaatgatcacaaagagatccacaccctgatatatcatggtaaaactgcttaaataaaaaaagagacaaaaacgaagggaaaattttgaaagcagcaaatatatgactaggtacatacaagagaactccaataagatcaacgtttgacttctcattagaaaccataaaggacagaaggcagtggagtggcatattctaagtgctgaaggaaaaaagaaactgtcaaccaagaatcatgcatccaacaaaactgtctttcaaaactgagataaagaaattcaaaaatacacaaaacttgagagaattgtttctagcagtgatgtcttacaagaaatattaaaggaggcacttcagaaataaatttanccaaaaatttgcaaaatgtatactttaaaaactataaaatgtcattgaaagaaattccagaagnccttaacaagtggaaaagcttcccatgttctgttcatggagcagaagacttattgttagtatggcagtgctctccaaattgatcaatagattcaatgcaaaagatgttcaacattagttattagagaaatgcaatcaaaaccacaatgagatacctctttacacccactacaatgactaaaattaaagacacacaataacaagtactggtaggaatgtggagaaattgaaaccctcattcattgctgttgggattgtaaaatggtgtagccactttggaaagcagattaggagttctgcaaaaggttgagtatatacttaccgtatgacccaacaatttcactactatgtatataccccaaagaaatgaaaacatgtacacacaaaatgttgtacatgaatgttcacagcagcattattcatagtagccaaaaagtggaaacaacccaactacttatcaactgatgaacagatcattgaaaacgtggtagacccaaaccgtgaaatatatatatatatatatatatatatatatatatagttgttgttgttgttgttgttgagatggagtctcactttgttgcccaggctggagtgcagtggtgtgatctcagctcactgcaacctccacctccaggctcaagcaactctcctgcctcagtctcccaagtagctgggattacaggcacgtgccaccgtgcccagctaatttttgcatttttagtagagacagggtttcaccatgttacccaggctggtcttgaactcctgacctcaggtgacccacccgcctcggcctcccaaagtgctgggattacaggtgtgacccaccgcacccagcccaaaccatggcatattattaaaccataaaaagtaatgaagtgctganacactgcacaatgacatggatggacttagaaaaggagctaagtataagaagccagtcataanacnctatatattgtatgattccatttatatgaaatgtccagaataggcaaattcatagagacagaaagtagattagtggtttccaggggctagaggaagtagngaatgaggagtgactgctaacaggtatgggttttctttnaagggtgatgnnnantctaaaatcagatcattgctgatggttgctgaagtctgtgaatatactaaaaatgattgatttgtatactttaaaagagtaaatgttatggtgtataaatcatatctcaataaagttgttacaaaaaagtaattaactagccatgtttgtttttgtttttctttgttttgttttttcatattgtcaaggaattcccagctttaagtcacaatcaagtgacaaagatagaactggggtggatgtatagtccattattaactactacttcttacttttctctagaataaaggataactatgagaggatactatagaaatcaatcttttattcttaaaaggaccattgctaatttttcactcacagtcatctctgggattgaattccatttatcaaatcccttttataagagcttttctagcttttagtaatggactggaataatttttatttcttagaatttctttctcttagtcagatttaagtgtaacctgctttgcttattcgtccatgtgttccaagtcatgattttgctcaggacaaataaaatgctctactagtatgagcctggtagaaagggaactaccatttaatgaggcctgtaatgtgtcagangctccactgatatttcccatccaatgcacacaacaaacctaggagcaggtattantgtcatctttttgaaggtgaagaagcaggctaatagaggtgaatggattgcccaacatcacacagtccaaagttgcagaccccgagaaagcctgtgttctttgctgaacaccaggctaccttccagcgttctcagttccgtgcaggtcagacggggttcacttatctttatggtcctttatagctctggtaaaaactaaaactgccagactttggctacttggagagttagttcaaggctgagggtgattttccaagtgcaatgcgtgatactcctcagcccaagaaccacattttccaggctgattttctatgactcctaattaattgttcaatattcattcaacaatttgttaagcacccactgtgtgcctagcactgtgcaaacactagtcatctaaaatatgattatacggattattcaggttttagaactctgtccttctgctggagnatggtgaggagggggtgcctctttcctctctagccaaagagcaagggtgaagtggcccagaacagcgcccaccccacccagggntgctggcctcatgcatgcacatgtgcacatctgtcctgtaaagtatgcttgnccttgagctttgtgagggcagggnccatgtctttnttgtgccctacacanagcctggcctggagcaggtaggtgctnaatgtcagttgaatcaatgattaagcaanttttttttttttttttttttttttttttttnaccaattcactaggagaacttgggctggttgctttattgctctgtgccttcatttcctcatttgtaaaacaaggataagagtacccatctcacatgccaaccctgagatcccctgagataatacacggcactcagtgagcactccacacactgttactgtctttcccattactccctttactatagggagccatgggaagcggcacatccttctatgccaagagtgacctcctatcccctattaccttgtcacctggatttttgtttcttaaattagagctgaaggccatgcggcccagctaaatggagctcactctgctctgttgtgagaaccttggtctgggaccctgctgatgggacagacatagtaaccccgggaggccccatcagaggggcagggagaggggaggtaagctatgtttcagaaatgttaggatttgacttgaaagcacacccccaaagcaatgccagccagctccttaccaggacatctgccattagcctctgagctggtcatgtgtatgtatgtgtgtgtgcatgtatgtacaaacatgtgcagggacccacagggacaggtctgtcccatcaaggaaaacactgtctcccactgggcccnccatgtcactgttaaccacagggaggttttgtggccctggctagcggagacccaagacatatccctcaaacctgaagggcccaggggcaggcaggnccggtttcctaagaaaagccntttctaaaggacccctcttaggactggcagctgatttactggactctagcccttcgctccaggtagaacttcatctgccaatcctgtgtcctaataccaggcccctcacagctttccaaaaagacacttgcggcaccataactccccaaaggagagcccagccctagaatcaaccatgggcagccatgcagtctagctgccttcccctgctggttgtaagcagggtctccaaaggcagatggcacccctcatgggcttggggcaaacggccactgcgtgtgcatggctcttcctgtgctgcatgatgctgacagggcttcttgggcccctcttcagggagggaatgggaccgaaagccatcgatgcagagcctacaggaaggagaatgatgggagatggccctcacctcaccccccttcccagtgtccccattcagaactcctgcatctgcttcatttcctggaggccaggtttgatcccctgccagggccagcttcatgggcatgcaactgtgcagtcacacagagcgccacacttggtttcaatgctttgccattgctgtattgaaattcttaataatttttgaccaaggagttctgcattttcagtttgccctggaccttgcaaattaagtagccagttcccagtcctaccctttgctcatgcaatgcctggctccacatctaacagccttccnttcctttctatctctaaatcctatctatcctgccctaaggcaaagtccagacagaaaaatatttgacagactacaaaggagagcacattatatttgaaagagcttttctgctcaactgcagatggaatgatgatggttggcatgttaatgcctctaccaatcttgagttcttcttggctcagtctgaggctaaaggctgtgatggaggccttctactcctagtcctgggtaattgtctcctggtatcacattccttgaagaantgtagggcatcatatttaataagctcattttgtcctgccttagggagtataggtccaaagcaggacactggtacaagattgggttctgaggcccaagtaggtatctatcatgctcatttacttgctatgacttcagacaaatcccattctcacagtctcagtttaatcttctgcaaaatgggtcacaccacatcatgaggctttgggcctcaattgctcatctctctaagtcagaagcagaacccaagcttttatctgaagctttggacaattatttttggagggagatggccatgaatgcaggcacacgtcctgtttaccacttccggtctatggactggcagaacacattaatctggccatacagttttagaggattcatttccctttctcctgaagcccatgcaagagccccaggcgaggcaatcctgctctgggtacagtctgagatttatccttggattgtgcctgagcagcagagcgctaacttcctggcccgagttgtttttcttctgcagagctgtgttgggcnttgcctgtggccagcatttgagcactgtgacaggcggcctcatgaatcttgctattgtctctaggacaggactattctcgaggacattatcctcagcatcacagagtggctcctttgtggaagaagtacataggagacacttctttcngatctcagtattcagaacaactgtgcttctgcgaataagcagactacttcggatactgtaacccttctgagagctgagctgctgggatacgcagagccagttttggagccccgtctggcaancaggcaggcccgtggacaggatagtctagggtcagtcttaccccaggcttggtttttgcaacttccctcctctttattttccattttccccaattcccttagctttttgttttacatttttctcttgtgtgtgtctttataaacacctctgtctgctgcagaatnaggtgtgatataaatatgtgagtttgtgcttaaatccaccacactttcaagggagttgtgaggattcagaaggaattgtttggcgaatgcagtaaaagcagccccatccaagatggagctagggctgggtgggaggcaggggtctttcctccagactggatcatctcgccacagacagcagcccccaaaacccaagcccaaagtgctcattggcatctgtttgtttatttatttgtttttaataaatgaaaggtcactaaacacactttgaaatttttgtcacctcccttgagatgttgcttaattgacttaagtcaaactgaaactctggctgaaactcattttgtagatgcactggcagggacttgttagggcagcagcatcccacagaccctagggaatgggagggaccacacttctggaaagactggatgggaaggagggtgtagggatgtaggaggggagctagggagccctggagaggagtaagtaggccccagaacgatggggccncgaatcaggccacaagcttggcctaggcgtgagtggaccagggaccacatgatttctcagaggggcctgctcagaaaataaagtcacagagctcctggaaccnnntgcacttttcaacaagggnaaagaaattttacgcctggaccaagtggccatgccctctgccctgccntttgggnttttcaaacacctnccctccgcactaggctggcttctgaagatgcagtgccacctttactttggactgcctcacagctgaggggtcaccagtcacagccatggccttggctggcttcttgctatgtaatgcgagcggagtaggagcatcaccctccctttccaaagaggatcctgagtttcagggaggncagtgacctgccccaggttactctccccagtcccgncacaggccagtcaaacccagggctgcaaggcctatgccctttgtacaaaggcaatatcctggcccaggggaggagagtctgcccgccagccgtgctaatattccctcagagctgggctggggttgaggtctgcctcagggttgngntcacgcttcatgctgtcttccttggcagtggaatctggtgtgtgaggacaactggaangtgcccctcaccacctccctgttnttcgtaggcgtgctcctcggctcnttcgtgtccgggcagctgtcagacaggtaagcacatgggaggggaggaaggtgggatgntgccccttgtcaatcactgcctatcttggggctggactgaattcagggtatttctacccnnnnnaagggaggaccctcagnccactgattcagtgataggaggagccccgatgtctcntattcaccccgccctcagtccccttcacctggacacanttttgttccattctcccagcctcatgctcccagggctttggccagtggtggggaggggccactgaccccactggaaagtggggtcctcataccacagactccttgacaaaacatgacgctgccatcaccactgccctccctgctccaccaccacctcaggggtaggggtggatggcacaggatctgtctgcaaacagccagcagccagacagtgagccccaactgtgcacacactgctgctgtggcttggaccctgggcnaggcactggggccacagagctgatgctggctctgaccctcatagagcttacaggcctgaggaggcagcatcacagcaccaggagacagtattccacaggcccctgtatgtgctgggtgaccagtgcaggaaggtgggggtactaaatggcaatgctggggaaagcctcccagcccaagccaacagccagagacatgagtttggaaaatggggcgggaaaggctgttctgctcacacctgtgctccattctatccactggtcactaccagaatgtgaagataatacttaaaacaacttcagtgtcaccaacagacattgctgaaagctacaccttcccacagagcagtggtttgcaatttttgttgcacgctactcatctgggaaactttcccaaatcctaatacccaaactgtatcccaaaatcttgggaggtggggtgctggcatcagtattttttccaagctctccgggtgattccaagtgcagccaaggttaaaaaccacttgcagagtgactcttagttctggatttacagaattttcatgttgctgattcaagaaatatctgcaatctgtgctgtacccactttccagaaatgtgctggtagcatctatctgtggctccttctaaagagcatatttccacaaggtccttgngtacactgctccatgcttttgagacatggtgtctccacaggaagctgagaatctcaaaagatatctcctgagcagatggatgcntgagacctcaccaaggaggaagatgggaggatgtgacagngaaaanagtctgccagagccctgaaaaaacactaantctgcattgatgcctgactcagggcttgcaacacaccttggcaacctacacatctcatgttttgtgttatactgcattctctaggtttggcaggaagaacgttctcttcgcaaccatggctgtacagactggcttcagcttcctgcagattttctccatcagctgggagatgttcactgtgttatttgtcatcgtgggcatgggccagatctccaactatgtggtagccttcatactaggtaggaatggcttctgggacatggggtgcttccctctaaccctctgaaaggcccagaaagaggaaatcattgggccattgggctgtgcttccaaagcctttggatatatgtgtcttgggagggagccgtagccacagctcttggagcaagtcaggggcagggcaggcactcgcagggagccacctctgtgctgagccctgaaatgtggtggagaagaagagcaggagccattaagaaccagctgcacaacagctgagacacattctctggagcacagggcttaagagaggcacagaggaggacagtttaggggaagggcagcctgaggatgtctttgatctgagttgtgtaggcttgagacttcttcctggttgcttgtgaagagacttagggtagtcactgtggccagtacacaggtggggaggctcatgtcagacagggggagttcagtgccagcccnccatcactgggcctatctggcctcaccctgagttagagcccaccccagagcctatcagctaagcacaggacaagcctccccacactgatagccccatctcctgtcaggctgcatgaggaagactcatcagttacaaggtcatgaagaggtccctggggccctcagcagcttttcctgggagggagttaggtgagctccaggcgagttgatgagggtcatccacaggagtgagagagtgtgttctcttgagcccagcgaatgggcaaatgagagaagcagaggtgaggggtatccctgggacagtagacaccagttcccatcatntctagggtgataatatattctaggtggctcagtaccatcccaatttgtccctattgtcacagtgtcattattaataatactcctttaatagaagtctaacgattcttcttgcctcaaaagtccgaatctggttaggtgactctgattgctttttaagagtctgaaggactcgttggctttggagatgctgcactgtcttaattgatggtgtgtacaccttctgtcaagttgccaggatggaaatattccaactctactcctttaattatttgcgaagagccccgtgccatgggctcagcctcagtgtctgcagaggggagaagggaggagacaaggtgggaagaacatgctggagccacatggtcccagcaggtagcacactcttcagcaggccccagcgggaggaggctgctccccttcctgcagggggaaggagcggggcaagattttcttgaagatcatttgtgtagagggcacagggagcttcaggccagtgttgttggaaactggaggttggaggaacttaagatagaaattgcaacctcctagtccccagtggctccaagaacacaaggcaaagcctattgtcttcatgttcaaagtccccagaatccatcttccntgtgacgatcctccctaagcctgaaaaacccaacatgtggctctgcttgggccagacacataccaaaagccaaacaggcctccccaaaacggaggactctgaccagcaggcccatatcccaggaggacctgaaggaccatctggaacagaagggtagagagtcacggtgaagccagagaagctctctccgtgggaatgggaacaaggtgggacttgtgccaggtggtggcatccccggttctatagaatgggcagcactgagatctgcctttttagtggcacagggtgattctggagcctccaagcctcccctntgtgagcgggtnaggcagggagagcataccccaggcttcctacaggaaagggctgccctcccccgccggctctgacactatcatctactctcctcccagttcccatgcagcatgtggtcccacatgcagggtgaagaagggtaagagggccaaggggaaagtaaggccatggaagatcagtcaagccagccagtcagtaggtaagaccctgggtgcagcagcctcgctatcagctgggatccaggcctctcagggcttgctatcagcctggggccaggcctgttatagggtgctctgggagcctcttcatggtagttttactccattgtgctctgcaggaaattactggcttttgtatcctggtatgagactcgttgtgttggnttctcaggtctgaaagtttaagtgtccctgaaaaccagcctgctctggaatggctggacgccccagtcagcacctggggttgggggacacagtacaaaggcaaggtggctgctagcttccctacagtgcagcagtgctctctggtgtttctgggataaaatagctgaattccaggttgtatttgactaaaataaaccaaagggtgtggtctnggctgaggagtttggtaggtgccttcgcttccancctctggccctaaggaagggttggcaaagctctgcatgataatttcacattaacttgggaagccaaattactcccactgtcaaagtagtgcagaaatgagtccttgttttgtgctgcttttaaaaaatgatcaaggctgccacagtcgtagcagactagaaaagagaaagaaagaacagtgctaccctacacagcataaagcagacatgcctggaagtgatggtgttgccggaatgatgctggtttaggaggaaatgaccactcgggaccaaggacactaaccctacccacgttccagctcccctcacaatctcctactgttgcccagaaataatgccctccacctgtcaatagccttctgggtagaatgccatcagtatcctctcttatcccccataactagctgcttaaacacaaaaagtgtttctgtntttaataacacagaaaaaactcttgactgattcattaattcagtatttttcagtacttctaacatcaacctccatgtgacatttctttgggcatgctgaaccatttaaagctttgcttcctctgatgtctagaggttcctccttttgataggtgtgtaagtgatggcatcttcctttacaactctggctaccaggaagctcaggcaaatttgaaatttaactacatcaatgatgcagcttttaccagccacatatatatatattttttactgtttttaaattgaacagttgaaatataattttgtcttcgcctgtttaggatgccgtaacaaaatcctataggctgggtggtttaaacaaccaaaatttattttccacagttgtggagggtagaagtccaagatcaaagtgccagcagggttggtgtctggtganggttctcccattgggttgcanatggctgccttctccctaagtcctcacatggtctttcctcngcgcaagtgctcagaaagagggaaagcaagaaagagagagtgagccatagagagctcactctntggtgtctcttcnngtaagaacactaatcctataccctatcaggtcaggggatcaatgtatgaataatagagggacacaaacattcagtccatgacagatttgcataccatgatattcacacttttaaagtgtataattcagtggtgtttagcatattcacaaagttgtacagccatcaacactatctaactcctcatcaccctgaaaagaaaccctatacgaattagcagtcatttccctcttcccctcgcccctggcaaccactaatttactttctgcctttaatggatttccctattccgcacatttgatataaacagaatcatatattatgtggcctttgtgactgactccacttgttttcaaggttgatccatgttgtaacatgaattagtattccactggatgaagacaacacatttcggtcatccattcatcagtcattgaacacctgggtgtttctactttggggctgttatgaataatgctatgaacattggagaataagtatttttgtggatatatgtttttaattctcttgggtataaggtaggacctctgctgggtcctatggtgattctatatttaaccntttgaggaactgccaaactgttttcaaaatagtcagctgcgtatttttgatcttttcaccaaggcacaactttttacttttctttatatcttactttaatgttaccattattttantgtatttaaatttgaagttgcctcaaaatctccaagggatgagctggcatataaatacgtaaacatgatgcagaaatttgctggcataccttatttgcattgcaccgtaagagggaagaactggggagacccagggaagagcaagaacttactcctgtctattcacaaggagctcactgcctggttgtgcctctgcagccaatgcagctgtcactgtggacatggggacatgggtcaagggagtggggcacatattgctgggtatacacagagataggaaatgttacttttgactggagtgattggtgcagacagaggggaagccctgagcaggggaacatctgttaagccctgaagaggagttctgtcttccactgggcctttttggatcaaaagcattgtactttccatcantcccctgacatgtagcacatttcaactgcttgatcaggataaagaataactattactgagcacctgccacatgccagacaccctcctaggcactttgcatacatgatctcagtcttcataataatatctccatgaaagacggcttatcacagtcactctagtagcaactcagcatcaaggaacccaggttctccccgtgccagatggaggacactcctagcccatggttcctgggtctcgcctcatatcctggcagctgcactacagtcttgtttcttgagctggggtctactgagagcctatgttggtcatttatgaaggtgcatgtcctgcctctgtgtggaatgggcccagcggttcctggggcaggtactgctctacatggaaggcagcctggccctctctgagtcactccctcttggagagagtgctcagattcagaggggtgtgacaggtggtgggcaatactcctagggctatgctggccctcatcctagccaggtcatgtccctttggcttagatgtcaaagtttcactcccaggagaaactcagaagcccagccagaccaagaccaaccaggctttgccctagaagcaaagcaggcagaagaggcagatagagcatagggggccaggacagggatcccccgtcagcacagcaggagtcactgctgtgtcatctgtttcactgccacatggaaagctcatggcagctgcaggccataaaagatctgtgtaatcagggttctctcctagcattactcagaggggtctggctggcttccagtgacaagccaaggaagggaaaatgtaaatccacttntgaaagggggataaggtgggtggatcacctgaggtcaggagttcaagaccagcctggccaacatggcaaaaccccgtctctactaaaaatacaaaaattagccgggcatggtggcaagcgcctgtagtcctagctacttgggaggctgaggcaggaaaatcgcttgaacccaggaggaagaggttacagtaagctgagattgcaccnctgcactctggcctaggcaacagagcacaagtctgtctcaaaaaaaaaaaaaaaagaaaaaaaagaaaaactgtaaaaatgacagcaggatanggccttacccatctcccctcatgtctntantggactctcagttgaagaactgctttataaggccaagtcatgcccccagtaatttttttcttggaaggtagaccagtatccatagagcaaataaggcttaaatcctggtgcaccacttattatgtgcatgaatttaagcaattggctttaactctgaagtctcagctaattatgtgtaaagtaggaggaatattagcttttgttttagtttttgtttttgtttgagatggagtttcgctcttgttgcccagtctggagtgcnnnggcntgatntcggctnactgcaacctctgcctcctgggttcaagcgattatcctgtctcancctcccgaatagntgggattacaggtgcctgccaccatgcctggctaatttttgtattcttagtagagacaggtttcatcatattggtcaggctggtctcaaactcctgacctcangtgatctgcctgccttggcntccctantgctgggattacaggcataanccaccgtgcctggcctggaatattagtttttatataactggtgtgaagggtcaaagagataatatatgtaaacacttancctgnaacctgtctcaaagtacctactcaaaaaaatgctanctgtgaagatggtgatcctntttaaggaagggtgactgcctaaaagagagcanaaagtangactaaaaanganttatttcaatttgtaccatccatgctgtccacaggaaggcaaagagagagacctacaaagtctctgtccccaacatgcactctgcaaagttatataactgttctggtctgagacccatgcttagagagggagattatccaggaacccagtagtataacttctcttttcttaacaaggtcatgaaggtaggagaaagctcctctggcctcacaattctacaagattctcacttgatcctgcgccctttctcttttccaaaatcaaaccctctgccactgaatctctgcttaagaataaagcaccttctatttatttccacatcacaatcacggccaaggggtttagaaaccagtgcatagcaggaaggtcattaatgaggtcagaaaagacctggaaatcatactcatgatcctcaaagaagtcaggccctatccttggaaatgggtttataggctgagcaatgctctgggtggttagggcaattgtctccaacatgggatgcaggaggcacaaagtactggctgtttctatcagtccaggtttttaaatggcctcttgagagggacccactgaggcagatctctcagttatgaacagtgaaggggaatgagtggggtgggatgggaacagataggtggtaaaatgagaaaccagccaacatagtaattttgctgcttctctggaattagctcttaatcccttagcctgtgcctagggcatataaacatgactgagaaaatctctcttaaaaggcangcatgaatgacctttgaattatctgttttgtatcttccaggccacttctctatagcatgggcagatccccaacaaaaggcagagccattgccattggtcacatctccgccccaggtgtagtgaaccaagggcagactctgtggaaagggcccagagctccgtgacaagagaaggagctttgtncttggactcccatctggtgccatctctgctcactgtggcactgagcagagcagaggccaatagtcgcctgcatgtcctttcccaaaaagcagtgcccgcaccaagcagtcatgggcctttctgctgttcagggaaagcactgtgagttcaatgggtgtgtggacaggtctagggctgctgcctgcaggggcagcagaacagcgcctgcttttctttccttggtccaaatggactgtggactgtgtctgcctgggctgatctctgggcacagcatgctgcaggaaggcctgtctgtgnacctccagagccagctcactccaactgatttctcagggctcaggcatgggtaagtgtgccctgaggtcttggaaatgatgaactctccactgcttcctgatgggccaatgtctgtttatttacagttcagaactgttggaaaggctacacacaccttgaccccacatggcccctcagaagagccactgcttgtctcctgaaatgctctctctgcttctcatgttggggctcttattttggaaatgtggcattctcagaagggattagctcttggggctgggcatgatatttttatgacatggaatggccttaaaataagttggccaggcgcggtggctcatgactgtaatcccaacattttgggaggccaaggcaggtagatcacttgaggtcaggagttcaagaccagcctggccaacatggcgaaactctgtctctactaaaaatacaaaaattagccaggcgtggtggtgcacgcctgtagttccagctgttcggaaggctgaagtgggaaggtcacttgagcttgggaggcagaggttgcaatgagctgagatcacgccactgcactgcagcccgggcaacagagggagactttgtctcaaaaataataataataataataaattaattaaaaataaataaataaatctatggctccattaaaatctcatagaaattcaaccttctataagttgccaagtcttggatcacgtcccaccttcctctaccctgaccatgcctgctctgggccatgaggcaaatgcctttccccttttctaagtggtgatagtttgaactctaactgccacataatgatataaaaagcaaatattaaagtgagttaatatgctaatactcctcccttgttttgaacaggaacagaaattcttggcaagtcagttcgtattatattctctacattaggagtgtgcacattttttgcagttggctatatgctgctgccactgtttgcttacttcatcagagactggcggatgctgctgctggcgctgacggtgccgggagtgctgtgtgtcccgctgtggtggtgagtgtgactcgtccccagacaggccctctgctgcgggtcagcacacctggaacacacctggagcctgatgctgacctagcctgggcttgcatgacctccacggaactcgcggaggccagcttccaagccgggagagtttttgcctcattgtgggtgggcctgtgtgctggagtttgagtctccattgtcctaaaaagcacattctcaggattccaatctcttccttgataccagccactcctgtctgttgctacttgttatttgtggagctggtggtgttggattttgttgtttttaactctgccccctgctctttcatcacttttctctctctagaaaaatctcactctatccaactaatgtaattnttcaagcttccctatcatataacgattagcatctatgaaagggaagcctatttattagcactatgatagtgtgcctatttatctgctatgtctcgaagagtgagaaaattactttttatttaataaattgttagagaaatgtcctcttctctccncccncaacctcccaactcagttttaacacaacagttataggaataacaaagtttgaagattctggcctcagtcttgcccaatatgactatctgggggatttcaggtgaaagaataaattaaaaatcatagaacaaggtgtcagacataccctctttttggagatttcctgttcctttgctttttcatatttaagcaagagaanaggagattccttccatnggtgggtctggttctgtctctctctcattctctttctctctcattaattcttctttgtttgatttcattatatcaaagtcaaatcttccagcactgactggtttcaccgaactgcataacttctacctgcttacacataacacaagcttctttgaccattttttggaactcacaatttagactagacctcatggttgccctgtcctttggatacctcctgactgggaagctgtctaggcctttatcttactgcagctgttaggcagtcatagaggttgacaggcaggccacctgtctgacgagatagcgcaggtcaggtgggctcactctaagcccctgtattctcatgacctgtaagagtgctgtgatttactagtgtaaattattggcatgaagtgtgcagggggagttgacagtttgttaaaggttatgtattattctgatttgcaacattcctctggaaccctgtctgaggaccnggaaacccggcctgaggagcantgaataagggctggccattatctacatagctgtgcctccaaggaaagcatcccagacttacaagagcagagcaattttctgaatgagagtaattatcatcatcatcatctgtccttctctccacaactcccttgctagctgaatttattggaaaagatgttagtgactaaggttctctcaagacacatgacaagattattcaaaggttcttttctgcctcttcagctgaatggttttaataaactatgttctctggctctagttgtcattttgacctaagagaccagtactttcaaaatgttataattagccaggtatggtgnttcacacctgtaatcccagcactttgggaggccaaggtgggaggatcactttagcccagcagttcaaaaccagcctgggcaacatggcaaaaccctgtctgtactaaaaataaaaaattagctgggtgcagtggtgagcgcctgtagtcccagctacttaggaggcagaggtgggagggtcccttgagcctgggagttcaagattacagtgngctatatgatcatgccactgcactccagcccagatagaatgagatgctgtctcaaaaaaaaagttataatcaatctgattaaggtatgattctatcagagagaaagagagagagagaataacaatataaagcctctgatgggctgtacagagattagtagggatgggatctttgcctcgggaccatatgtcacagcgaggaacgaacagctctgatccattagccctccagagtcagcctccagagtttccagcagagaaggtccatcccacagggagcctgctcactntgccctaaggagagctttttcttctattccccaggagctggtccatgaatgctcctggctgtgggcatactgtaccagggacaggacttgccccagacagaggccagcctgggaccagcactgacaagctgagtgctccccctgaacatttaccagtggtcttaccattttcctataaaagaaaatagctttcttctgaacatcccatccttgcaggtgggtcttnccaaagggatgctcatcannctannggntggggtntggagggatgcttagagctgacatggaccctgccgcctgctcagtatgagncaagctctccagcaacacctcactgctgtntagtctganttcaatctacagagagattatagtgtgctacctcatttggcactccattgacttttgaaatgatcagctagatttgagattatgtgcaccaaaaaaaaatcaaagcaattcaaaactagagataccagtggccctgagtacagtgggctagaaatgggaggtaaacctacatagaggcactgctggtggtttgggggactttttatgtcccaagtttcctttaaaaggcttggcagtaagtggccacaggtgtgaatggagtatgtgtataggcggtggggatggggtagggaagcaggccatagatgcaatgtgaggaaagctatgcaatttctagctgattaaacattaagaatctgaggccagtggtctgtagaaaaaaaggtaggatatttctaagcataatctggttggagaaagatactggccccatccatgaggtatatcgtggctgtgtagtaagatctctaattctgcactaatatagtagccaccagccacatgcagctatttaagttaaaattaattaaaagtaaataaaattgaattttcatttcttcaatcacactagctcaatagctacatgtggttagtgggtgctgtcttagtacagatacagaacatttcagaagtttctatcggacagcacagttctaattactgagagtcagtgacctaacattatngtagttcctatagggacccactaaaggcatattctgaaaaatgcaaactaggctcatttttcaactcatgtgctattaagctggagaggggacctgctggttggtcatttgttttgctctgagctaattcctagtgtgggtttggggccaattaactacacaggactctgtgtctgctacttataatttgttntctaggctaagtaagtacagatgctctttgacttataatggggttacatcccaataagcccattgtaaattgagaatattctaggtcaatacacctaacctactgaggtaggaggccagacacgactccagaggcagggcttggacactggaccaaattgaggaccagctaaaacanagccagggcagaagacgctttccataagacacgcccacccgtgtgccatgtcagtttactgttgccatgacaacactcaggctttattgcccctttccatggcaatgacccaatgacccaaaaattactaccctttccctagaaatttctgcataaactgacctttcatctacatgtaattaaaagtagttataaatatgagtgcaaaactgtcctgagctgctactctctgcctgtggggcagccctgctctgcaggagcagtcacggagctgtaacattgcntcttcaataaagctgttttcttccacctccagtttgcccttgaattctttcctgggcaaagccaagaaccctcatgggccaaaccccactctggagcttgcctgccctgcatccctaccaaatatcatagctcagcctgacctaccttaaacatgctcagaacacttacattagcctacaatngggcaaaattttctaacacaaaacctattttataattaagtgttgaatatttcatgtaatttattaaatactgtactgaaagtgaacaacagaatggctgtatgtatgagcactcaaagtaaggtttctactcaatgcatatcatttttctcctatagtagaaaaatcataagtcgaaccatcataagttagggaccatctgtactgacaataggtaaggaaaatgagagctgacaggagaatggctaaaagattttgagaaataaaaatttagaaagaatagaagccctattatttgcaactgtgtttgagcatcagctataaatgggaatagactatgtaatttaaaacaataagcttttttagacattaacaaaaagacaacattggggctcatctcccaaaagctggtttgggcactcgctgatctgggaatctgttgaaagccatgggggccccacacaggngggtgtcagtgccctcactttctcttttgactaaaggaaacgatcangaacacaggtgccctggccatccttttcccaataaactgtgaacatcaccagctgggacaccagcaattctccttaggatggacccactgcaaagacagtctcttctctaggggaacttatagtctagtagctatgtttcttttatgagccacactacctataagttttggatgaacttctccagagccctggctcaaacatgcccatagttaatgtcaccccttatggtctggtcctggtctcaagcacaactgatgatcggtcttataatttgatcagaagtttgaaacttgaaggtggttttatttattcctatgctccaatagaaatcttatgtggactccctttaaaaagacaaactagaattttacaaaattaaaactatttgggggaaactcagatttaatagtatccagccctgctgttgtgaaaaattattaaatattaaaaataaattataggttcattcctgaatctccccgatggctgatatcccagagaagatttagagaggctgaagatatcatccaaaaagctgcaaaaatgaacaacanagctgtaccagcagtgatatttgattctgtggaggtaagcatttgcagatgtttcctcttgagatcagctatgcattcttgattttatggaatttaacttaattcaatatttgttaanngcccactatgtaccaggcattgcactaggccttaggaacacaatcatgattaaattctaagataaaatccaccttaaagggctgtttataagaattaaatgcaataatatatgtaaactgcttagcaaatagcaagcgttcattaaatattagtttgctttattttgcaagtttatcatcaactttctattggcattgctccaaaccactgacacagctatgtactgatgatttttaaatgtccagattaagtggaaaaatatgtttcagggctagtccaccctgggttctccactgtaccctgaatcttaccacagactgctgaggaagctatgcatgtctttagtctcactctgtgtcatgtctagaaccttaaggcctgaaagcacaggcttgaggattcacccttgaaagctggtggccacaaggaaaagctgatattctgtggaagcagccncacctcctgacagttcgtgaggatatgcagtgtgccatgccctgtgctggctcctagaggaaataagaaaagacacagcccaaattctaaaggaacatagaatgtaactaagattggcaagaggattagataaaaagataaagaatggcataagggaggaaatggtaagtatccaagtagagacactgtgtgccagcaatacagagaaagatagcagggtggcccatggctggaggcaggtccaagccagggaggacagcaggactcacttaaatgagtggagaggatggggcagggcattcctgtctaggagataggtaggagtctgctcaggacacctaaaaaagactgtggtggcttcagtaacattatacatgccctgcaccaaagcatgtagtacagaaagcagaactctgtagaggacggtgggcatgccactgctcacccccacggcttccccagtccctggccttcaggaccttttccagaagcagatactctggctatggctagaggcttagactggccatgaactcaagctctcagagggctgccaggacaaaacatcccttaatcctttgcccttcaaatcaagaagtccatttcaagtctcagtcaatttaaatggataaaacaacaacaacaacaacaacattgggagtgtctaacacttctgcccttcagggctgtgctggggtgtttatnggtatttatcagctaggtggcctgattcctatctgtgaaacttanagtctagttgtagggatctacctatgtggatgacagcaatcataaaatagatatgagatatatagttatggacaaataagaatatagttcagtgttaaaacacatggttctggcagtgccttnananacacacacacacacacncananananannnnnannnatatntnnntntntntatatatntntntntntatntntatatatanananacacactttaaaaaaattttgagtctcactgttttgctcaggctagagtgcagtggtacaattacagctcaatgtagcctcgacctcgaccttctgggctcaagcaatcctcccacctcaacctcccaagctgggactacaggcacttgccaccaaacccagctaacattttgtatttttttttgtagagacagggtttcactatgttgcccaggctggtctcaaactcctgggctcaagcaatcctcctacctcagcctcccataatgctaggattacagatgtgagccatcattctcagcctgacagtgccttctttcttcctgccatgatgcacatctagcctgaactggacaaacccattaagggaaagggggcttgcgctttcctgaggcctggtcatctcccctgctcacctcccgtaggatcctctttaggaggatctgcagagccctgtgccagcaaatgcaccctatttagaatgatattgaagagcctaccaaatgccaggcactgtgttctgctcattgctcatgcttttttttttttnnaaaaaannaannnangnnnnagcgaagttaagtaacttgcccaggatcacacagatgttaaactgggatttgtgacctagttctcccaggcaccaaagtacatgctcttaacaactgtgatttttcacttcatgttacaggcagtagcattaacttggataggaaaagatatagagtagagtattgtcttagaaagcnatctcaagcatccttggcttcanaagaagcaccttcaacctgctnctggtacctgatccaaagacattttggtggctccacccaggccagggacaaccactggatacaagcatattcttgttctgaggtttccaatcctggctgctcatcacaatcacccaagtgctttaagaatacagattctcaggcctctgtttaaaagagtcgaagtaggcaagcccagggaggaacctggcaacctgcatttaagaagggccccgtggaaccaccaagtattagcacaggactggtccacaggtcctgtctgtgtgcaccaccctggaaccctccaagangatgtctggttcccactagtttatgcagtggctttgctttgttataataccagggttattctgctgggggcatgttctggggctctacaatccaaatagtgcaagagagaaaaggcaatgtggctgngagctgtgtaagtgtctgatcattcctagaaaaaaggacactcacatttggagaggacctgctgtgaaggcaggcctggcactcccacttcttcataataaaaactcctccatcagtctcttactgcgcatgcactgtagggccacaaagtcacaaagtcctggtacttcatccctgagagattccccacctcagagcctaataacttttatgttagtttcaattctaatcagcttggttaccttgattcataactcatagcaagttaatatcagcaaattaaaagcaaaagaataggccgggcgcagtggctcacacctgtaatcccagcactttgggaagctgaggtggatggatcacctgaagtcaggagtttgagaccagcctggccaatatggcgaaaccccatctctactaaaaatacaaaaattagccaggtgtggtggcaggtgcctgtaatcccagctactcaggaggctgaggcaggggaattgcttgaacctgggaggcggangttgcagtgagctgagatcgcgccactgtactccagcctgggcaacaagantgaaactccctctcaaaaaacaaacaagaaaataaatataaataaaagtaaaagagtataaacccattgatagcagtaaggatttgtcactgaccgctgatggctagcctcttttcctcaatgatacagtaataataataatagtttaaatttactggacatttaccatggttcaagcactgtgctaagctatatatatatgatctcatttaatctctcaactcccaattaatatacccattttatatagcagtaagctgaagttcagagaggatcgttacttgcccaaggcccgccagtaaactgaagtctgttctgatgctaaatgactacaacaagagaagtgtaaaaagcaaggctgggtaatcccagcactttgggaggccaaagtgggcagactgcttgaggccaggagtttgagaccaacctggccaacatggtgaaacccagtctctacaaaaaatgcaaaaattagccagatgcggtggtgctcgcctgtaatcccagctactcaggaggctgagaggcaggagaatcgcttgaacctgggaggtggaggttgcagtgagctgagatcacaccactgcactccagcctgggccacagagctagactccatctaaaaaaaacaaacaaataaacagagaagtgtaaaaagcagacgtggcctcaaggaagggactacatggccctacccttcaataggcagtcctgaggatcacagatcagtgtggtaaatgacatccattctctgactgtcagtcagagaaaggctatgttagccaacaggctttgtctaacgagacagggaagggggtgttatatatttaaaaggcttctgtgtaaaatggactacaaacattttaaagtgtcagagtaagtgtcatatatgtagatgccctattgcctccaatcaccagacttctaagcatagtcaaggtgaagtgngagttgcatgcataagttttccccatgcatcatagccaaagatacttccttactacctcttaatctcatggttatttgcattattttggttacaggagctaaatcccctgaagcagcagaaagctttcattctggacctgttcaggactcggaatattgccataatgaccattatgtctttgctgctatggtaantaataagtgacctggaaatgcagatatccagcacataagtatgcaactgatttncttaactcagagnaacattatgacaacgactgggttntcctgaggaaaaattaagattataaaattctaaattattttgaaatgcctctattcagttcatgggactagatattgaaaaatgtcactgggcgcantggcttacgcctgtaattccagcactttgggaggccaaggtgggcagatcacctgaggtcaggagttcgagacgaatctagccaacatggtgaaaccccgtctctactaaaaatacaaaaattacctggncatggtggcacatacctgtaatcccagctacttgggaggctgaggcaggagaatcacttgaacccaggaggtggaggttccagtgagccttgatcgcactactgtactccagcctgggcagcagagcaagactccatctaaaaaaaaaaaagtcacagggtacagtatttaagtctgcagtactgggacccaaagggaaagcattcccttccccctcctaaagttatgggattgagatcattgatgtccctgggtaatggttccctccccaccaaaaattatgagattaagactaatgatgtccgtgttgcctgaggcaganatgctaattcagctcctgcttcctgaagctgacctggcaattgaacactgaggctagaataacctagatccccattgcctggattggagccttgatcagctttttacacgtgccaatagcaaactaacaagtggctttcaggcgcagaaatactgaattggctttatagattagatttaataatttatctgccnnnaaaaaaggaaaaaagaaaantcctactctatgatggcagcacacacacacacacacacacacacacacacacacacgatgatngtcagaactggtagatttaggctgcagctaatctaagtttatttttaagggccatcctacttggcaattagaagagatcaatgacagagcttatgcgtctgaattaagcaagtgtgtcctaggtacatgggcttnttagttcttacatttcagcctatatccctgatgcaaatgttcttgggtagtngtgctactaagctaataaaaaattagtcactagttctatctaanctactcctattactaaatgggaaaatcactaagatcccttgagcatacttatttgttgtgtttatgaattagtccttgtattatctaatttgggtctagtatgtgtaaaaagatccactagtgttcctgaagtcatttttcagatatcctctgcttctcagtcatgttaataataagcaaatttctacacaggggtgtctgcatagtgcagttgtaacaaaaactcaggttcagtcactcacagcttgcagcgtccaattaacaagagcaaagtctgatgtaaggaaagtgactttttattccaaagctagcttagggagaaaggtatcggcttcctgcctgaagggtaccaccgctcctctttttgaagcggaaagcaggcacttctaaaaggcaggggaggacgcgagcaggtgagggatcaganggctagcttgctgccttatctactgggcagttganntggtgccttcatgggcagaaacaggttgtataggtggccaaaaactctagcaggcatactttgagttggaaattgactgatatcgcttgaggaaatctgggcngtgagagttccactctggagcttctaagcacatagttagatgaatttgccctgcagggagtgtccagtgaagaggaggttaaaaggctataattgcacttctaaagagctaagtagaaagtgcagaaaaggaggaaagagaaaangagagagaaaataaactatctcttagaaaaatgggggtactcagttacatagttatttttccacagagaagcttcttttttttatttatgtttttatttttttgaganagagtttcgctntgtcacccagcctggagtgcaatggtgcgatctcagctcactgcaacnttcgcctcctgggttcaagcaattctcctgtctcagcctcccaagtagctgggattataggcacatgccaccaccatgcntggctaatttttgtcttttaagtagaganggggtttcaccatgttggccaggctggtctcaaactcctgacctcaggtgatttgcctgcctcggtctcccaaagtgctgggatttcaggcatgagccaccacgcatggcccacagagaagcttctacatggaagttaactataatgacagaatatacagactgcagtctgtgatactttgaaggattaggggacattcactcttttccctaaagagaatcagaatttattctaagtgaatcccacagtatgacccttgtatctcttttttgttaaagaatgaaatctggattttatgttcactgatgtgactaatattttttcttcatataatagctctaagagattgaggaggtacaaactattcaggaaataggaaaacttgaaatcaactagctgagaaaagttattgttgaatattggtatatctagactagcactgtccnatagaactttctgcagtgacggaaatgtttcatatctatgtngttcaatatagtagccactagctacacgtggctattgagcacttgaagtgtggctagtgtaactaaggaactgaatttgtaatcttatttaattttaatttattcaaatttaagtagccaatgagggtagtggctanttataagacagcaagatatagagaatttcttgaccatcataaaattttagagcgattcacaccatccctttgtcatttttaccttcttctttcaggatgctgacctcagtgggttactttgctctgtctctggatgctcctaatttacatggagatgcctacctgaactgtttcctctctgccttgattgaaattccagcttacattacagcctggctgctattgcgaacnctgcccaggcgttatatcatagctgcagtactgttctggggaggaggtgtgcttctcttcattcaactggtacctgtgggtaagaagttaaccaagatgaacagcttaccagaaaagacccacatattgactaggctgcttctgagtcactgctccatgtttattcagcctttacatcataagccattcataagtcaattagacaggtgattgctcactacgtatgttgtttcctgtgttcaaaacaactaccccaaacagaagagacagtctcagagaagcagaaagcaccaaagaggaagagtaactctcccagtcatccccttcacagaannactcacctaacatctccctttaactcatantnntgattcctaatttntggctactctcaaacattttccctgggtgtatattcaagtattacagtgagtaggacatgtcggttccaggatacattcagtggttttctggtctgaatctttaagtagttaaatagcacacaaaaggatatatgcttcccttttccccttgtgtctgacgttctcataatattaaaatgtcactaaattattgatagtaatagaaatacatatttcttaataataagaaagaggatagaaaggattggtcctcctctttgaagataaaacttaactatattaacatgctaatagnttacccatgaaactgttttaacaaacaaaagacaagtggacaaaggtcactgaatgcattaatgacctagaataacagtagaacaaagtacaaagccacccctcacatgagctccatctttgtccatgcccaaatattaggacatttgaagagaaaatgctattttgggaatatatttatgttaataaaagtactcccactgaagcaaaaaggacaataaaaataactgatataagaaagtatcanttctaaaagcaccattgtttataccgggtctcttttccagattattacttcttatccattggtctggtcatgctgggaaaatttgggatcacctctgctttctccatgctgtatgtcttcactgctgagctctacccaaccctggtcaggaacatggcggtgggggtcacatccacggcctccagagtgggcagcatcattgccccctactttgtttacctcggtgagctgcatcttgtgcatttgttcttcctttaaattagttttcaatgntaaaagtaagattttcattgtgaaaacgtcaaatagcatagaatgtacnggaaanaaagtacaagttcncctctcctctcccaggaggagctctagaaagccnatcncaaactggtgagtagngntncagacantttgttttgcacnacatcatgtncncangtatgtatataattttttaaacacacnngcaactcaggcgtggtggctcangcatgtaatcccagcactttggnaggccaaggtaggtggatcacctgaggccaggagttcaagaccagcctggccagcatggtgaaaccctgtctctactaaaaataaaaaaattagccaggtgtgctggcaggtgcctgtaatcccggctactctggaggctgaggtaggagaattgcttgaacccaggaggcagaggctgcagtgagtcaagattgcaccactgcactccaacctgggcaacaanagcaaaactctgtctcaaaaataaataaaaataataaaaaataaaaagaataaacacacaaataacattatatatgttaaaactttttaaacttaacattgtatcagaatatccttccaaatagttacatattgctcctttaccttctttttaaataggtacctccttttaaaaatgtatataagtacattttaaaataagtatatgtaagtataatctacagatagattcacagaagtaaaattcctggagcaaagagtgccgtntaaaaaattatatatatatgtgtgtgtgtacataatgttgtgcaaaacaaaatgtctgtagggctactcaccaaacagttcatgttggttttctagggatcctcctgggagaggggaggtgaacttgtacttttttccagtacattccatgccatttgaagttttcacaatgnaaaatcttacttttatactngaaacacataatttatttaaccaggctccaatgaatggaggacatgtaggtcatttccaggtttttgctattacaaataancattacaacgaacattctncctatgctcctacaccctttgtgaatttacttacgtatatttggatttacaggtagattcatagaagtaaaattcctggagcaaagagagccattaaaaaatttcatagatattcccaaattgaactctacatattgtgccaattataatctaattattaataggtaagagtggttttcaataatctcaccaacaataaagattacctaattcttttttttttttttttttctgagacagggtctcactctggagtgtagtggtgcgatcctagattcccagctcactgcagcctcaatcccccaggctcaagagatcctcccacctcagactcccaactagctggaactacaggcatgcacaccgtcaagcccagctaatttttttttttttctggtanagacgggattttgccagtttgcccaggctggtttcnaactcctgggctcaagtgatccacccgcctcaacctcccaaagtgctgggattacaggtttgagccacagcacctggcctaattttttaatcattggttatctgttatgtgaaaaatggatatcattgttactttcattcctatttattaattatgaatgaggttgagcatgtttttgtgtattttttggccattttatttatttttcagtgaactgactgctcatgttctttgcccctttttttttttttggagatggagtctcactctgtcacccaggctggagntacagtggcgcaatctcagctcactgcaacctccgccgcctgggttcaatcaattctcctgcctcaacctcctgagttgctgggactacaggcgcatgccaccatgtccggctaattttttntttttttttgtatttttagtagagatggggtttcatcatattggtcaggctggtctcaaacccctgacctcaggtgatccacctgccttggcctcccaaagtgctgggataacagacatgagccactgtgcctggccttctttgcccattctttaatggtgtatatcgggcaaaattcagccctgataattcacataggnttcttttctattttccctaagtgccagctggtctgagaaataaagggacagagtacaaaagagagaaattttaaagctaggtgtccgggggagacatcacatattggcaggttccatgatgccccctgagccataaaaccagcaagtttttattagtgatttcaaaaggggagggagtgtacgaatagggtgtaggtcacagagatcacgtgcttcacaaggtaatagaatatcacaaggcaaatggaggcagggtgagatcacaggaccacaggaccggggcaaaattaaaattgctaatgaagttttgggcacgcattgtcattgataacatcttatcaggaaacagggtttgagagcagacaaccggtctgaccaaaatttattaggtgggaatttcctcatcctaacaagcctgggagtgctacgggagactggggcttatttcatccctacagctncaaccgtaaaagacagctgcccccaaagtggccattttagaggcctaccctcagggacgaattgtctttctcagggatgttccttgctgagaaaaagaattcagcgatatttctcccatttacttttgaaagaagagaaatatggctctgttctgcccgactcaccagcggtcagagtttaaggttatctctcttgttccctgaacattgctgttatcctgttcttttttcaaggtgcccagatttcgtattgttcaaacacacatgctctacaaacaatttgtgcagttaacgcaatcatcacagggtcctgaggcgacatacatcctcctcagcttatgaagatgacgggattaagagattaaagacaggcataggaaatcacaagggtgttgattggggaagtgataagtgtccatgaaatcttcacaatttatgttcagagattgcagtaaagacaggcgtaagaaattataaaagtattaatttggggaactaataaatgtccatgaaatcttcacaatccacattcttctgccatggcttcagccagtccctcctttcggggtccctaacttcctgcaacaggtgtattcatctttttctttttgacttgtgcaaactctttgcaatggaagaaattagccccttgtatatatgttgtaaatatttttcctagtttaccatttgtattttcactttatgatatctttgtaatgaagacaattttagtttttatatactcaaaggtgtcaatctgaaatgaaactacagaggtgtttcagcggcatgttcaatctgatgtaggatctgacaccccatgcctggtcctcattaaggactctactggtgagaaatgcagacacacctcatcaattgggcaattggttacctactggtacacacgtacacacacacacacacacacacacacacctgaaaacatgtatgaattctcaggaaaggtataatccaaacccttacagagaaagagaaacattttcctaacaaacatattctgcacagttgccctgtgccacacctgagatagcagattatacagagtggtgttatttttccctttgtccttccctgtactttcaaactatttcaaactatgctcagatggtccagtggtcagacactggtacataagacagggagtccaagagtctcctactccacacagctactcctgttagtgacacagaggcctcagagaagctgcaataacaatattttccttatgttttgggtcaaatgaaatccatgggtgtgctgttcctctgtaagctctgtatgtatacaggggcaaagcaagatgaggatctgtttttctcctgtgtgagtaaagcttcacaaagagattacatattaacccaagacatgtaagtgctccaaaaagaagccaaatccaggcacacagtgcctttacagaatgtgcctgatacttataatttactgatttaatctgaagtgaacataatacttagttgttttttttttttttttttttttttttgagtctcatagccctgtggacctaactttccactgcagcctactagtttgggcaaaagcagagtcatgccagacctatcatngccaaaacctatccctgaaacatcctggttaacctccttctccttgatgtcctcccatcccccatttccaattacctccaccttaagaggagaactctggtaggcaaagaactctaccaaaggaacatctgtcacacatgactgagtagtagcttagaggataccagttgacaggaaagaatgaaaagccttatgataatgcaaaaggattatagacatgcacaatgtcatctgccaaaaagaaactgatattttgcttacttttttttctctgaaaatgttcagatttaaatccattctcttatactgttcaccaacttcacaaaatgatgctcaagagtgcccagagagtcctcctatctgattgatgttcttatgtcccgggctttacaggtgcttacaacagaatgctgccctacatcgtcatgggtagtctgactgtcctgattggaatcytcaccctttttttccctgaaagtttgggaatgactcttccagaaaccttagagcagatgcagaaagtgaaatggtaagtaggacttttaacaaaatgataccaaaatgccttagggagaataagcatggaagaatagctaggaaggttctgaaatagaagagtaataagtagagactagctctatcagataataaagcatattgttggctgggcacnnngnnnnncncnnntnnnnncnnnncnntnngngnggctgaggcaggtggatcacctgaggtcaggaatttgagaccagcctggccaacatagtgaaacctcatctttactaaaaatacaaaaaattagctgggtgtggtggcagtcncatgtaatcccagctactcaggaggctgaggcaggagaactgcttgacccaggaggcagaggttgcagtgagctgagatcacaccactgcactccagcctgggcaacaagagcaaaactccgtctcaaaaaaaataaataaataataggccaggcgcggtggcacatgcctgtaatcccaacactttgggaggctgaggcaggcggatcatgaggtcaagagatcgagaccatcctggccaacatggtgaaaccctgtctctactaaaaatacaaaaattagctgggcatggtggcacacacctgtagtcccagctactcaggaggctgaggtaggagaattgcttgaacccgggaggtggagnttgcagtgagccaacatcatgccacngcactccagactgggcaacagagctagactctgtctcaaaaataataataatagtaataaagcatattgttgagcaatagtaattaaaacaatgtgataatggtataggaatagaaaccaatggaataggatagagttcaggataaaacccaagaaaatataaaaattcagtatgtaataaaaagtagcaaagtcaagggggaatagttattcagtaaaaggtattagaacattcagctagacattttgaataaattaaaaagttgtctctttaaaaaatgcattggctgggcatggtggctcacacctgtaatcccagcacttagggagattgaggcaggcagattgcttgaggtcaggagttcgagaacagtctgtccaacatggtgaaaccccatctctactaaaaatacaaaaattagccaggcatggtggcgcacccctgtaatcccagctcgcgggaggctgaggcaggagaatcgcttgaaccagggtggtggatgttgcagtgagccgaaatcgcgccactacactccagtctgcgtgacagagcgagactccgtctccanaaaaaaaacacatcaagataaaatccagacagttaaaaaatttaggcctaaaaatgtaaaatcataaatataccaggaggaaacaaacatttttataaccttagagaagatcttactgagcataacataaaatcaaagaaccataaaggaacagattgatatatctgattgtacattttttaaattattggcaaggcaaaaagaaaccacccacaaagacaaaagtcaataagccaggagaaaatatttgctacacaaaacctaaacataaggctaatatacttaatatacaaaaagctcatagtaaccagtgagaaaaagacaaacaattcagtaggaaaaccagcaaaggacaagaacagacagtttacagaagaagtcacttcagtgtccaatatatacatgaaaaaagttatttgtccacattaataaggaaatgcaaaaataaaacaattcaacatctttttttggcttatgagattgcgcaagggtttttcttcttgacgcaaaccaattctgacacctattgagtgtcctataatgcagtncaattttgacaatacctaaatttagcatcagactccacaggtgtaaagactcagtcccacaatactgccttgcttcacttgccactcacaaatgtcaggtccccaggttacccacatttctctctgactttgctaccaagtctggggttccaacaaatcccctccttgggtttgattatttgctagaatgactcacagcactcagaacactggccactagcngtttcttacaaaggatacaaatgaacagccagatgaagaggtacatacagagaggtctggaagggtcctgagtgcagaggttctgtccctacggagttaaggtgcactgccctcctggcatgtggatgtggtcaccaactcggaagttctccaaaccccatcatttagaggttttcatggaggtttagtcatgttggcatgatggattattaagtctatccccagcctctctcctctccccagaggttccaggcttctaatccaggcttagtcttctggtgaccagcccccaccctgaagctatctaagggcccagccaagggttgcctcattagaacaaaagaagcttttatcactccctatcactccagaaattataagagatttaggatctctatgttaggagccagggacaaagaccagtatctttttatgacaccacagcaaacatttaaaagattagtggtatttaggagaagtttagagtttcgagtaacactgcctggctttaaatcccaaatctactgcaaaaatttgggcatatcacttaatattcactaagcttcagtattcccatctgctatatgggatatttcatacggctattttgaggttataaaagataaagtgttcccagtgcctggccttgataaaggtaactctgcatatgaattaattctgatccctgacagaacatttatagcaaaaagtgcccattgttgataagttgttttcttctaatgatagaactacttcagtatcaactttaattgccaagaatttcatgcctccctctctatcctttgcttgggaaaactgtgaaagagtttaaattctggcttctatgcttaattaagaaagtagcctcagatgtttaaaagcataaaatattagatctacaccatgaggttctcattagagttcaaatagttacttgttcattgattggagaaatatttttcaatgtcaatttggatggagcattttgaggaggaaagtcttgaatatttaattttccttagcatctattttattttcaggttcagatctgggaaaaaaacaagagactcaatggagacanaagaaaatcccaaggttctaataactgcattctgaaaaaatatctaccccatttggtgaagtgaaaaacacaaaaataagaccctgtggagaaattcgttgttcccactgaaatggactgactgtaacgattgacaccaaaatgaaccttgctatcaagaaatgctcgtcatacagtaaactctggatgattcttccagataatgtccttgctttacaaaccaaccatttctagagagtctccttactcattaattcaatgaaatggattggtaagatgtcttgaaaacatgttagtcaaggactggtaaaatacatataaagattaacactcatttnccaatcatacaaatactatccaaataaaaataacatcattgtattaacgcaaatattaggtgacaacnaatgtgtgtgtatgtgtgtgtgtttgtacatgtggaagtgaaccagcacagtcaaaattatagtagtttctgaagtgaaccaaatgattatatgcagnattcctatccagaaaaccttccacactcctttgagagggagtgatcactgttagaggaaaaaacaaaccactttttaccctgtactgtcacactcaacccacaatacttctgtgaccagatgtggtggggggcttctcatacaccaagtaattctccagcagataccaatggggtgttctctaatagaattaagctctgaccctatctgcggggagatagtgtcagatcccacaggttgtgtgctctgtccctcttcagatgccattcnaacatccaagccacacgcacctctgaccta